(19) United States
(12) Patent Application Publication
(10) Pub. No.: US 2015/0284394 A1
(43) Pub. Date: Oct. 8, 2015

(54) INHIBITORS OF BRUTON'S TYROSINE KINASE

(71) Applicant: HOFFMANN-LA ROCHE INC., Nutley, NJ (US)

(72) Inventors: Niala Bhagirath, Bloomfield, NJ (US); Romyr Dominique, Ridgewood, NJ (US); Joshua Kennedy-Smith, New York, NY (US); Francisco Javier Lopez-Tapia, Honolulu, HI (US); Eric Mertz, Fair Lawn, NJ (US); Qi Qiao, San Diego, CA (US); Sung-Sau So, Verona, NJ (US)

(21) Appl. No.: 14/438,008

(22) PCT Filed: Oct. 23, 2013
(86) PCT No.: PCT/EP2013/072123

§ 371 Date: Apr. 23, 2015

Related U.S. Application Data
(60) Provisional application No. 61/718,746, filed on Oct. 26, 2012.
(60) Provisional application No. 61/831,443, filed on Jun. 5, 2013.

(51) Int. Cl. C07D 487/04; C07D 519/00

(52) U.S. Cl. CPC C07D 487/04; C07D 519/00

(57) ABSTRACT

This application discloses compounds according to generic Formula I wherein all variables are defined as described herein, which inhibit Btk. The compounds disclosed herein are useful to modulate the activity of Btk and treat diseases associated with excessive Btk activity. The compounds are further useful to treat inflammatory and auto immune diseases associated with aberrant B-cell proliferation such as rheumatoid arthritis. Also disclosed are compositions containing compounds of Formula I and at least one carrier, diluent or excipient.

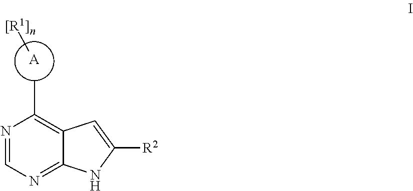

### FIELD OF THE INVENTION

The present invention relates to the use of novel compounds which inhibit Btk and are useful for the treatment of auto-immune and inflammatory diseases caused by aberrant B-cell activation.

### BACKGROUND OF THE INVENTION

Protein kinases constitute one of the largest families of human enzymes and regulate many different signaling processes by adding phosphate groups to proteins (T. Hunter, Cell 1987 50:823-829). Specifically, tyrosine kinases phosphorylate proteins on the phenolic moiety of tyrosine residues. The tyrosine kinase family includes members that control cell growth, migration, and differentiation. Abnormal kinase activity has been implicated in a variety of human diseases including cancers, autoimmune and inflammatory diseases. Since protein kinases are among the key regulators of cell signaling they provide a target to modulate cellular function with small molecular kinase inhibitors and thus make good drug design targets. In addition to treatment of kinase-mediated disease processes, selective and efficacious inhibitors of kinase activity are also useful for investigation of cell signaling processes and identification of other cellular targets of therapeutic interest.

There is good evidence that B-cells play a key role in the pathogenesis of autoimmune and/or inflammatory disease. Protein-based therapeutics that deplete B cells such as Rituxan are effective against autoantibody-driven inflammatory diseases such as rheumatoid arthritis (Rastetter et al. Annu Rev Med 2004 55:477). Therefore inhibitors of the protein kinases that play a role in B-cell activation should be useful therapeutics for B-cell mediated disease pathology such as autoantibody production.

Signaling through the B-cell receptor (BCR) controls a range of B-cell responses including proliferation and differentiation into mature antibody producing cells. The BCR is a key regulatory point for B-cell activity and aberrant signaling can cause deregulated B-cell proliferation and formation of pathogenic autoantibodies that lead to multiple autoimmune and/or inflammatory diseases. Bruton's Tyrosine Kinase (Btk) is a non-BCR associated kinase that is membrane proximal and immediately downstream from BCR. Lack of Btk has been shown to block BCR signaling and therefore inhibition of Btk could be a useful therapeutic approach to block B-cell mediated disease processes.

Btk is a member of the Tec family of tyrosine kinases, and has been shown to be a critical regulator of early B-cell development and mature B-cell activation and survival (Khan et al. Immunity 1995 3:283; Ellmeier et al. J. Exp. Med. 2000 192:1611). Mutation of Btk in humans leads to the condition X-linked agammaglobulinemia (XLA) (reviewed in Rosen et al. New Eng. J. Med. 1995 333:431 and Lindvall et al. Immunol. Rev. 2005 203:200). These patients are immunocompromised and show impaired maturation of B-cells, decreased immunoglobulin and peripheral B-cell levels, diminished T-cell independent immune responses as well as attenuated calcium mobilization following BCR stimulation.

Evidence for a role for Btk in autoimmune and inflammatory diseases has also been provided by Btk-deficient mouse models. In preclinical murine models of systemic lupus erythematosus (SLE), Btk-deficient mice show marked amelioration of disease progression. In addition, Btk-deficient mice are resistant to collagen-induced arthritis (Jans son and Holmdahl Clin. Exp. Immunol. 1993 94:459). A selective Btk inhibitor has been demonstrated dose-dependent efficacy in a mouse arthritis model (Z. Pan et al., Chem. Med Chem. 2007 2:58-61).

Btk is also expressed by cells other than B-cells that may be involved in disease processes. For example, Btk is expressed by mast cells and Btk-deficient bone marrow derived mast cells demonstrate impaired antigen induced degranulation (Iwaki et al. J. Biol. Chem. 2005 280:40261). This shows Btk could be useful to treat pathological mast cells responses such as allergy and asthma. Also monocytes from XLA patients, in which Btk activity is absent, show decreased TNF alpha production following stimulation (Horwood et al. J Exp Med 197:1603, 2003). Therefore TNF alpha mediated inflammation could be modulated by small molecular Btk inhibitors. Also, Btk has been reported to play a role in apoptosis (Islam and Smith Immunol. Rev. 2000 178:49,) and thus Btk inhibitors would be useful for the treatment of certain B-cell lymphomas and leukemias (Feldhahn et al. J. Exp. Med. 2005 201:1837).

### SUMMARY OF THE INVENTION

The present application provides the Btk inhibitor compounds of Formula I, methods of use thereof, as described herein below:

The application provides a compound of Formula I,

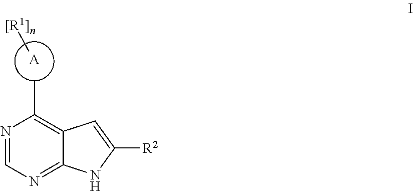

wherein:
A is phenyl or piperidinyl;
each R¹ is independently halo, lower alkyl, CH₂NHC(═O)R¹, CH₂N(CH₃)C(═O)R¹, CH₂NHC(═O)CH₂NHR^{1′}, CH₂R^{1′}, or CH₂NHR^{1′};
n is 0, 1, or 2;
R^{1′} is phenyl, unsaturated or partially unsaturated bicyclic or monocyclic heteroaryl, or heterocycloalkyl, optionally substituted with one or more R^{1″};
   each R^{1″} is independently lower alkyl, halo, cycloalkyl, heterocycloalkyl, loweralkyl heterocycloalkyl, oxo, cyano loweralkyl, hydroxyl loweralkyl, or lower alkoxy;
   R² is H, R³ or R⁴;
      R³ is C(═O)OR^{3′}, C(═O)R^{3′}, or C(═O)NH(CH₂)₂R^{3′};
         R^{3′} is H, lower alkyl, heterocycloalkyl, amino, or OH;
      R⁴ is lower alkyl or heteroaryl, optionally substituted with one or more R^{4′}; and
         R^{4′} is hydroxyl, amino, OC(═O) CH₂CH₃, or C(═O)OH;
            or a pharmaceutically acceptable salt thereof.

The application provides a compound of Formula I,

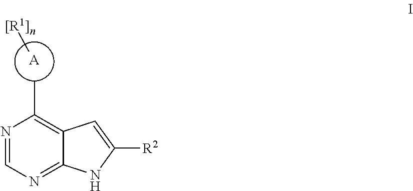

wherein:
A is phenyl or piperidinyl;
each R¹ is independently halo, lower alkyl, CH₂NHC(═O)R¹, CH₂N(CH₃)C(═O)R¹, CH₂NHC(═O)CH₂NHR^{1′}, CH₂R^{1′}, or CH₂NHR^{1′};
n is 0, 1, or 2;
R^{1′} is phenyl, unsaturated or partially unsaturated bicyclic or monocyclic heteroaryl, or heterocycloalkyl, optionally substituted with one or more R^{1″};
   each R^{1″} is independently lower alkyl, halo, cycloalkyl, heterocycloalkyl, loweralkyl heterocycloalkyl, oxo, cyano loweralkyl, hydroxyl loweralkyl, or lower alkoxy;
   R² is H, R³ or R⁴;
      R³ is C(═O)OR^{3′}, C(═O)R^{3′}, or C(═O)NH(CH₂)₂R^{3′};
         R^{3′} is H, lower alkyl, heterocycloalkyl, amino, or OH;
      R⁴ is lower alkyl or heteroaryl, optionally substituted with one or more R^{4′}; and
         R^{4′} is methyl, hydroxyl, amino, CH₂—CH₂N(CH₃)₂, OC(═O) CH₂CH₃, CH₂C(═O)OH, CH₂CH₂OH or C(═O)OH;
            or a pharmaceutically acceptable salt thereof.

The application provides a method for treating an inflammatory and/or autoimmune condition comprising administering to a patient in need thereof a therapeutically effective amount of the compound of Formula I.

The application provides a pharmaceutical composition comprising the compound of Formula I, admixed with at least one pharmaceutically acceptable carrier, excipient or diluent.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “------” drawn through a bond each refer to the point of attachment of a functional group or other chemical moiety to the rest of the molecule of which it is a part. Thus, for example:
MeC(═O)OR⁴ wherein

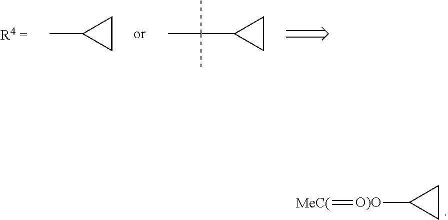

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds of Formulae I may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)=CH—), amide/imidic acid (—C(═O)—NH—⇄—C(—OH)=N—) and amidine (—C(═NR)—NH—⇄—C(—NHR)=N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl”, “haloalkylheteroaryl”, “arylalkylheterocyclyl”, “alkylcarbonyl”, “alkoxyalkyl,” and the like. When the term “alkyl” is used as a suffix following another term, as in “phenylalkyl”, or “hydroxyalkyl”, this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “spirocycloalkyl”, as used herein, means a spirocyclic cycloalkyl group, such as, for example, spiro[3.3]heptane. The term spiroheterocycloalkyl, as used herein, means a spirocyclic heterocycloalkyl, such as, for example, 2,6-diaza spiro[3.3]heptane.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “ester” as used herein denotes a group of formula —C(═O)OR wherein R is lower alkyl as defined herein.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl” or “hydroxyalkyl” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “PCy₃” refers to a phosphine trisubstituted with three cyclic moieties.

The terms “haloalkoxy” or “halo-lower alkoxy” or “lower haloalkoxy” refers to a lower alkoxy group, wherein one or more carbon atoms are substituted with one or more halogen atoms.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to a cycloalkyl composed of 3 to 7 carbons in the carbocyclic ring.

The term “carboxy-alkyl” as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic or partially unsaturated ring containing four to eight atoms per ring, incorporating one or more N, O, or S heteroatoms, the remaining ring atoms being carbon, with the understanding that the attachment point of the heteroaryl radical will be on an aromatic or partially unsaturated ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, oxazinyl, pyrrolyl, pyrazolyl, imidazolyl, oxazolyl, 4,5-Dihydro-oxazolyl, 5,6-Dihydro-4H-[1,3]oxazolyl, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole, naphthyridinyl, 5,6,7,8-Tetrahydro-[1,6]naphthyridinyl, and benzisothiazole. Bicyclic moieties can be optionally substituted on either ring, however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, and ionic forms thereof, unless otherwise indicated. Examples of heterocyclic radicals include, but are not limited to, morpholinyl, piperazinyl, piperidinyl, azetidinyl, pyrrolidinyl, hexahydroazepinyl, oxetanyl, tetrahydrofuranyl, tetrahydrothiophenyl, oxazolidinyl, thiazolidinyl, isoxazolidinyl, tetrahydropyranyl, thiomorpholinyl, quinuclidinyl and imidazolinyl, and ionic forms thereof. Examples may also be bicyclic, such as, for example, 3,8-diaza-bicyclo[3.2.1]octane, 2,5-diaza-bicyclo[2.2.2]octane, or octahydro-pyrazino[2,1-c][1,4]oxazine.

### Inhibitors of Btk

The application provides a compound of Formula I,

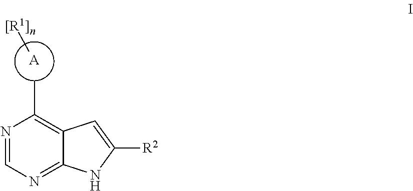

wherein:
A is phenyl or piperidinyl;
each R¹ is independently halo, lower alkyl, CH₂NHC(═O)R¹, CH₂N(CH₃)C(═O)R^{1′}, CH₂NHC(═O)CH₂NHR^{1′}, CH₂R^{1′}, or CH₂NHR^{1′};
n is 0, 1, or 2;
R^{1′} is phenyl, unsaturated or partially unsaturated bicyclic or monocyclic heteroaryl, or heterocycloalkyl, optionally substituted with one or more R^{1″};
   each R^{1″} is independently lower alkyl, halo, cycloalkyl, heterocycloalkyl, loweralkyl heterocycloalkyl, oxo, cyano loweralkyl, hydroxyl loweralkyl, or lower alkoxy;
   R² is H, R³ or R⁴;
      R³ is C(═O)OR^{3′}, C(═O)R³, or C(═O)NH(CH₂)₂R^{3′};
         R^{3′} is H, lower alkyl, heterocycloalkyl, amino, or OH;
      R⁴ is lower alkyl or heteroaryl, optionally substituted with one or more R^{4′}; and
      R⁴ is methyl, hydroxyl, amino, CH₂—CH₂N(CH₃)₂, OC(═O) CH₂CH₃, CH₂C(═O)OH, CH₂CH₂OH or C(═O)OH;
         or a pharmaceutically acceptable salt thereof.

Further it is to be understood that every embodiment relating to a specific residue A, R¹, R^{1′}, R¹⁴¹, R², R³, R^{3′}, R⁴ and R^{4′} as disclosed herein may be combined with any other embodiment relating to another residue A, R¹, R^{1′}, R^{1″}, R², R³, R^{3′}, R⁴ and R^{4′} as disclosed herein.

The application provides a compound of Formula I, wherein A is phenyl, R² is H and n is 1.

The application provides a compound of Formula I, wherein R¹ is halo, R² is H and n is 1.

The application provides a compound of Formula I, wherein R¹ is halo.

The application provides a compound of Formula I, wherein R² is H and n is 2.

The application provides a compound of Formula I, wherein R² is H, n is 2 and one R¹ is halo.

The application provides a compound of Formula I, wherein R² is H, n is 2, one R¹ is lower alkyl.

The application provides a compound of Formula I, wherein R¹ is CH₂NHC(═O)R^{1′}.

The application provides a compound of Formula I, wherein R¹ is CH₂NHC(═O)CH₂NHR^{1′}.

The application provides a compound of Formula I, wherein R¹ is CH₂NHR^{1′}.

The application provides a compound of Formula I, wherein R¹ is CH₂NHC(═O)R^{1′}, R² is H and n is 1.

The application provides a compound of Formula I, wherein R¹ is CH₂NHC(═O)CH₂NHR^{1′}, R² is H and n is 1.

The application provides a compound of Formula I, wherein R¹ is CH₂NHR^{1′}, R² is H and n is 1.

The application provides a compound of Formula I, wherein n is 2, one R¹ is CH₂NHC(═O)R^{1′} and R² is C(═O)OR^{3′}, C(═O)R³, or C(═O)NH(CH₂)₂R^{3′}.

The application provides a compound of Formula I, wherein n is 2, one R¹ is CH₂NHC(═O)R^{1′} and R² is lower alkyl or heteroaryl.

The application provides a compound of Formula I, wherein R^{1′} is tert butyl or halo.

The application provides a compound of Formula I, wherein R^{1′} is tert butyl or halo, R¹ is CH₂NHC(═O)R^{1′}, R² is H and n is 1.

The application provides a compound of Formula I, wherein one R¹ is fluorine and R^{1′} is tert butyl.

The application provides a compound of Formula I, wherein one R¹ is fluorine and R^{1′} is tert butyl, n is 2, one R¹ is CH₂NHC(═O)R^{1′} and R² is C(═O)OR^{3′}, C(═O)R^{3′}, or C(═O)NH(CH₂)₂R^{3′}.

The application provides a compound of Formula I, wherein one R¹ is fluorine and R^{1′} is tert butyl, n is 2, one R¹ is CH₂NHC(═O)R^{1′} and R² is lower alkyl or heteroaryl.

The application provides a compound of Formula I, wherein A is piperidinyl.

The application provides a compound of Formula I, wherein A is piperidinyl and n=1.

The application provides a compound of Formula I, wherein A is piperidinyl, n=1 and R¹ is CH₂NHC(═O)R^{1′}.

The application provides a compound of Formula I, wherein A is piperidinyl, n=, R¹ is CH₂NHC(═O)R^{1′} and R^{1′} is phenyl optionally substituted with one or more R^{1″}.

The application provides a compound of Formula I, wherein A is piperidinyl, n=, R¹ is CH₂NHC(═O)R^{1′} and R¹ is phenyl optionally substituted with one or more lower alkyl.

The application provides a compound of Formula I, wherein A is piperidinyl, n=, R¹ is CH₂NHC(═O)R^{1′} and R^{1′} is phenyl optionally substituted with tert butyl.

The application provides a compound of Formula I, wherein A is phenyl.

The application provides a compound of Formula I, wherein A is phenyl and n=1 or 2.

The application provides a compound of Formula I, wherein A is phenyl, n=1 or 2 and one R¹ is CH₂NHC(═O)R^{1′}.

The application provides a compound of Formula I, wherein A is phenyl, n=2, one R¹ is CH₂NHC(═O)R^{1′} and the other is halo and R¹ is phenyl, unsaturated or partially unsaturated bicyclic or monocyclic heteroaryl, or heterocycloalkyl optionally substituted with one or more R^{1″}.

The application provides a compound of Formula I, wherein A is phenyl, n=2, one R¹ is CH₂NHC(═O)R^{1′} and the other is F and R¹ is phenyl optionally substituted with one or more R^{1″}.

The application provides a compound of Formula I, wherein A is phenyl, n=2, one R¹ is CH₂NHC(═O)R^{1′} and the other is halo and R^{1′} is phenyl optionally substituted with one or more lower alkyl, halo, cycloalkyl or heterocycloalkyl.

The application provides a compound of Formula I, wherein A is phenyl, n=2, one R¹ is CH₂NHC(═O)R^{1′} and the other is halo and R^{1′} is phenyl optionally substituted with one or more lower alkyl or halo.

The application provides a compound of Formula I, wherein A is phenyl, n=2, one R¹ is CH₂NHC(═O)R^{1′} and the other is F and R¹ is phenyl optionally substituted with one or more lower alkyl.

The application provides a compound of Formula I, wherein A is phenyl, n=2, one R¹ is CH₂NHC(═O)R^{1′} and the other is halo and R^{1′} is phenyl optionally substituted with one or more tert butyl.

The application provides a compound of Formula I, wherein A is phenyl, n=2, one R¹ is CH₂NHC(═O)R^{1′} and the other is F and R^{1′} is phenyl optionally substituted with one or more tert butyl.

The application provides a compound of Formula I, wherein A is phenyl, n=2, one R¹ is CH₂NHC(═O)R^{1′} and the other is F, R^{1′} is phenyl optionally substituted with one or more tert butyl and R⁴ is heteroaryl optionally substituted with one or more R^{4′}.

The application provides a compound of Formula I, wherein A is phenyl, n=2, one R¹ is CH₂NHC(═O)R^{1′} and the other is F, R^{1′} is phenyl optionally substituted with one or more tert butyl and R⁴ is heteroaryl optionally substituted with one or more methyl.

The application provides a compound of Formula I, wherein A is phenyl, n=2, one R¹ is CH₂NHC(═O)R^{1′} and the other is F, R^{1′} is phenyl optionally substituted with one or more tert butyl and R⁴ is heteroaryl optionally substituted with methyl.

The application provides a compound of Formula I, wherein A is phenyl, n=2, one R¹ is CH₂NHC(═O)R^{1′} and the other is F, R^{1′} is phenyl optionally substituted with one or more tert butyl and R⁴ is pyrazolyl optionally substituted with methyl.

The application provides a compound of Formula I, wherein A is phenyl, n=1, R¹ is CH₂NHC(═O)R^{1′} and R^{1′} is phenyl optionally substituted with one or more tert butyl.

The application provides a compound of Formula I, wherein A is phenyl, n=1, R¹ is CH₂NHC(═O)R^{1′}, R^{1′} is phenyl optionally substituted with one or more tert butyl and R⁴ is heteroaryl optionally substituted with one or more R⁴.

The application provides a compound of Formula I, wherein A is phenyl, n=1, R¹ is CH₂NHC(═O)R^{1′}, R^{1′} is phenyl optionally substituted with one or more tert butyl and R⁴ is pyrazolyl optionally substituted with one or more R^{4′}.

The application provides a compound of Formula I, wherein A is phenyl, n=1, R¹ is CH₂NHC(═O)R^{1′}, R^{1′} is phenyl optionally substituted with one or more tert butyl and R⁴ is pyrazolyl optionally substituted with one or more methyl.

The application provides a compound of Formula I, wherein A is phenyl, n=1, R¹ is CH₂NHC(═O)R^{1′}, R^{1′} is phenyl optionally substituted with one or more tert butyl and R⁴ is pyrazolyl optionally substituted with methyl.

The application provides a compound of Formula I, wherein A is phenyl, R² is H, n=2, one R¹ is CH₂NHC(═O)R^{1′} and the other is F and R^{1′} is phenyl optionally substituted with one or more halo, lower alkyl or cycloalkyl.

The application provides a compound of Formula I, wherein A is phenyl, R² is H, n=2, one R¹ is CH₂NHC(═O)R^{1′} and the other is F and R^{1′} is phenyl optionally substituted with one or more Cl, tert butyl or cyclopropyl.

The application provides a compound of Formula I, wherein A is phenyl, n=1 and R⁴=heteroaryl optionally substituted with one or more R^{4′}.

The application provides a compound of Formula I, wherein A is phenyl, n=2 and R⁴=heteroaryl optionally substituted with one or more R^{4′}.

The application provides a compound of Formula I, wherein A is phenyl, n=1 and R⁴=heteroaryl optionally substituted with one or more methyl.

The application provides a compound of Formula I, wherein A is phenyl, n=2 and R⁴=heteroaryl optionally substituted with one or more methyl.

The application provides a compound of Formula I, wherein A is phenyl, R² is H, n=2 and R¹ is CH₂NHC(═O)R^{1′}.

The application provides a compound of Formula I, wherein A is phenyl, R² is H, n=2, one R¹ is CH₂NHC(═O)R^{1′} and the other is halo.

The application provides a compound of Formula I, wherein A is phenyl, R² is H, n=2, one R¹ is CH₂NHC(═O)R^{1′} and the other is F.

The application provides a compound of Formula I, wherein A is phenyl, R² is H, n=2, one R¹ is CH₂NHC(═O)R^{1′} and the other is F and R^{1′} is phenyl.

The application provides a compound of Formula I, wherein A is phenyl, R² is H, n=2, one R¹ is CH₂NHC(═O)R^{1′} and the other is F, R^{1′} is phenyl and R^{1″} is lower alkyl or cycloalkyl.

The application provides a compound of Formula I, wherein A is phenyl, R² is H, n=2, one R¹ is CH₂NHC(═O)R^{1′} and the other is F, R^{1′} is phenyl and R^{1″} is tert butyl or cyclopropyl.

The application provides a compound of Formula I, wherein A is phenyl, R² is H, n=2, one R¹ is CH₂NHC(═O)R^{1′} and the other is F, R^{1′} is phenyl and R^{1″} is tert butyl.

The application provides a compound of Formula I, wherein A is phenyl, R² is H, n=2, one R¹ is CH₂NHC(═O)R^{1′} and the other is F, and R^{1′} is unsaturated or partially unsaturated monocyclic heteraryl.

The application provides a compound of Formula I, wherein A is phenyl, R² is H, n=2, one R¹ is CH₂NHC(═O)R^{1′} and the other is F, and R^{1′} is unsaturated or partially unsaturated bicyclic heteraryl.

The application provides a compound of Formula I, wherein A is phenyl, R² is H, n=2, one R¹ is CH₂NHC(═O)R^{1′} and the other is F, R^{1′} is unsaturated or partially unsaturated monocyclic heteraryl and R^{1″} is lower alkyl.

The application provides a compound of Formula I, wherein A is phenyl, R² is H, n=2, one R¹ is CH₂NHC(═O)R^{1′} and the other is F, R^{1′} is unsaturated or partially unsaturated bicyclic heteraryl and R^{1″} is lower alkyl.

The application provides a compound of Formula I, wherein A is phenyl, R² is H, n=2, one R¹ is CH₂NHC(═O)R^{1′} and the other is F, R^{1′} is unsaturated or partially unsaturated monocyclic heteraryl, R^{1″} is lower alkyl and R⁴ is heteroaryl optionally substituted with one or more R^{4′}.

The application provides a compound of Formula I, wherein A is phenyl, R² is H, n=2, one R¹ is CH₂NHC(═O)R^{1′} and the other is F, R^{1′} is unsaturated or partially unsaturated bicyclic heteraryl, R^{1″} is lower alkyl and R⁴ is heteroaryl optionally substituted with one or more R^{4′}.

The application provides a compound of Formula I, wherein A is phenyl, R² is H, n=2, one R¹ is CH₂NHC(═O)R^{1′} and the other is F, R^{1′} is unsaturated or partially unsaturated monocyclic heteraryl, R^{1″} is lower alkyl and R⁴ is heteroaryl optionally substituted with one or more methyl, hydroxyl, amino, CH₂—CH₂N(CH₃)₂, OC(═O) CH₂CH₃, CH₂C(═O)OH, CH₂CH₂OH or C(═O)OH.

The application provides a compound of Formula I, wherein A is phenyl, R² is H, n=2, one R¹ is CH₂NHC(═O)R^{1′} and the other is F, R^{1′} is unsaturated or partially unsaturated bicyclic heteraryl, R^{1″} is lower alkyl and R⁴ is heteroaryl optionally substituted with one or more methyl.

The application provides a compound of Formula I, wherein A is phenyl, R² is H, n=2, one R¹ is CH₂NHC(═O)R^{1′} and the other is F, R^{1′} is unsaturated or partially unsaturated bicyclic heteraryl, R^{1″} is lower alkyl and R⁴ is heteroaryl optionally substituted with methyl.

The application provides a compound of Formula I, wherein A is phenyl, R² is H, n=2, one R¹ is CH₂NHC(═O)R^{1′} and the other is F, R^{1′} is unsaturated or partially unsaturated bicyclic heteraryl, R^{1″} is lower alkyl and R⁴ is pyrazolyl optionally substituted with methyl.

The application provides a compound of Formula I, wherein A is phenyl, R² is H and n=1.

The application provides a compound of Formula I, wherein A is phenyl, R² is H, n=1 and R¹ is halo or lower alkyl.

The application provides a compound of Formula I, wherein A is phenyl, R² is H, n=1 and R¹ is Cl, F or methyl.

The application provides a compound of Formula I, wherein A is phenyl, R² is H and n=2.

The application provides a compound of Formula I, wherein A is phenyl, R² is H, n=2 and one R¹ is halo and the other is lower alkyl.

The application provides a compound of Formula I, wherein A is phenyl, R² is H, n=2 and one R¹ is Cl or F and the other is methyl.

The application provides a compound of Formula I, wherein A is phenyl, R² is H, n=2 and both R¹ are methyl.

The application provides a compound of Formula I, selected from the group consisting of:
4-(4-Chloro-phenyl)-7H-pyrrolo[2,3-d]pyrimidine;
4-(3-Chloro-phenyl)-7H-pyrrolo[2,3-d]pyrimidine;
4-(2-Chloro-phenyl)-7H-pyrrolo[2,3-d]pyrimidine;
4-(3-Fluoro-4-methyl-phenyl)-7H-pyrrolo[2,3-d]pyrimidine;
4-(2,4-Dimethyl-phenyl)-7H-pyrrolo[2,3-d]pyrimidine;
4-(3,4-Dimethyl-phenyl)-7H-pyrrolo[2,3-d]pyrimidine;
4-p-Tolyl-7H-pyrrolo[2,3-d]pyrimidine;
4-(3-Chloro-4-methyl-phenyl)-7H-pyrrolo[2,3-d]pyrimidine;
4-tert-Butyl-N-[4-(7H-pyrrolo[2,3-d]pyrimidin-4-yl)-benzyl]-benzamide;
3-Chloro-N-[4-(7H-pyrrolo[2,3-d]pyrimidin-4-yl)-benzyl]-benzamide;
2-(3-Chloro-phenylamino)-N-[4-(7H-pyrrolo[2,3-d]pyrimidin-4-yl)-benzyl]-acetamide;
4-tert-Butyl-N-[2-fluoro-4-(7H-pyrrolo[2,3-d]pyrimidin-4-yl)-benzyl]-benzamide;
4-{4-[(4-tert-Butyl-benzoylamino)-methyl]-3-fluoro-phenyl}-7H-pyrrolo[2,3-d]pyrimidine-6-carboxylic acid tert-butyl ester;
4-(4-((4-tert-butylbenzamido)methyl)-3-fluorophenyl)-7H-pyrrolo[2,3-d]pyrimidine-6-carboxylic acid;
4-tert-butyl-N-(2-fluoro-4-(6-(morpholine-4-carbonyl)-7H-pyrrolo[2,3-d]pyrimidin-4-yl)benzyl)benzamide;
4-{4-[(4-tert-Butyl-benzoylamino)-methyl]-3-fluoro-phenyl}-7H-pyrrolo[2,3-d]pyrimidine-6-carboxylic acid dimethylamide;
4-{4-[(4-tert-Butyl-benzoylamino)-methyl]-3-fluoro-phenyl}-7H-pyrrolo[2,3-d]pyrimidine-6-carboxylic acid methylamide;
4-{4-[(4-tert-Butyl-benzoylamino)-methyl]-3-fluoro-phenyl}-7H-pyrrolo[2,3-d]pyrimidine-6-carboxylic acid (2-hydroxy-ethyl)-amide;
4-{4-[(4-tert-Butyl-benzoylamino)-methyl]-3-fluoro-phenyl}-7H-pyrrolo[2,3-d]pyrimidine-6-carboxylic acid (2-dimethylamino-ethyl)-amide;
4-tert-Butyl-N-{1-[6-(1-methyl-1H-pyrazol-4-yl)-7H-pyrrolo[2,3-d]pyrimidin-4-yl]-piperidin-4-ylmethyl}-benzamide;
4-tert-Butyl-N-{4-[6-(1-methyl-1H-pyrazol-4-yl)-7H-pyrrolo[2,3-d]pyrimidin-4-yl]-benzyl}-benzamide;
4-Cyclopropyl-N-{4-[6-(1-methyl-1H-pyrazol-4-yl)-7H-pyrrolo[2,3-d]pyrimidin-4-yl]-benzyl}-benzamide;
4-Isopropyl-N-{4-[6-(1-methyl-1H-pyrazol-4-yl)-7H-pyrrolo[2,3-d]pyrimidin-4-yl]-benzyl}-benzamide;
N-{4-[6-(1-Methyl-1H-pyrazol-4-yl)-7H-pyrrolo[2,3-d]pyrimidin-4-yl]-benzyl}-4-oxetan-3-yl-benzamide;
4-(3-Methyl-oxetan-3-yl)-N-{4-[6-(1-methyl-1H-pyrazol-4-yl)-7H-pyrrolo[2,3-d]pyrimidin-4-yl]-benzyl}-benzamide;
4,5,6,7-Tetrahydro-benzo[b]thiophene-2-carboxylic acid 4-[6-(1-methyl-1H-pyrazol-4-yl)-7H-pyrrolo[2,3-d]pyrimidin-4-yl]-benzylamide;
4-tert-Butyl-N-{2-fluoro-4-[6-(1-methyl-1H-pyrazol-4-yl)-7H-pyrrolo[2,3-d]pyrimidin-4-yl]-benzyl}-benzamide;
6-tert-Butyl-N-{2-fluoro-4-[6-(1-methyl-1H-pyrazol-4-yl)-7H-pyrrolo[2,3-d]pyrimidin-4-yl]-benzyl}-nicotinamide;
5-Methyl-thiophene-2-carboxylic acid 2-fluoro-4-[6-(1-methyl-1H-pyrazol-4-yl)-7H-pyrrolo[2,3-d]pyrimidin-4-yl]-benzylamide;
4-tert-Butyl-N-(2-fluoro-4-{6-[1-(2-hydroxy-ethyl)-1H-pyrazol-4-yl]-7H-pyrrolo[2,3-d]pyrimidin-4-yl}-benzyl)-benzamide;
4-tert-Butyl-N-{2-fluoro-4-[6-(1-methyl-1H-pyrazol-4-yl)-7H-pyrrolo[2,3-d]pyrimidin-4-yl]-benzyl}-N-methyl-benzamide;
5-Methyl-thiophene-2-carboxylic acid {2-fluoro-4-[6-(1-methyl-1H-pyrazol-4-yl)-7H-pyrrolo[2,3-d]pyrimidin-4-yl]-benzyl}-methyl-amide;
2-tert-Butyl-5-{2-fluoro-4-[6-(1-methyl-1H-pyrazol-4-yl)-7H-pyrrolo[2,3-d]pyrimidin-4-yl]-benzyl}-4,5-dihydro-thieno[2,3-c]pyrrol-6-one;
5-tert-Butyl-isoxazole-3-carboxylic acid 2-fluoro-4-[6-(1-methyl-1H-pyrazol-4-yl)-7H-pyrrolo[2,3-d]pyrimidin-4-yl]-benzylamide;
N-{2-Fluoro-4-[6-(1-methyl-1H-pyrazol-4-yl)-7H-pyrrolo[2,3-d]pyrimidin-4-yl]-benzyl}-4-(3-methyl-oxetan-3-yl)-benzamide;
4-(Cyano-dimethyl-methyl)-N-{2-fluoro-4-[6-(1-methyl-1H-pyrazol-4-yl)-7H-pyrrolo[2,3-d]pyrimidin-4-yl]-benzyl}-benzamide;
4,5,6,7-Tetrahydro-benzo[b]thiophene-2-carboxylic acid 2-fluoro-4-[6-(1-methyl-1H-pyrazol-4-yl)-7H-pyrrolo[2,3-d]pyrimidin-4-yl]-benzylamide;
N-{2-Fluoro-4-[6-(1-methyl-1H-pyrazol-4-yl)-7H-pyrrolo[2,3-d]pyrimidin-4-yl]-benzyl}-4-(1-hydroxy-1-methyl-ethyl)-benzamide;
3-tert-Butyl-isoxazole-5-carboxylic acid 2-fluoro-4-[6-(1-methyl-1H-pyrazol-4-yl)-7H-pyrrolo[2,3-d]pyrimidin-4-yl]-benzyl amide
3-tert-Butoxy-azetidine-1-carboxylic acid 2-fluoro-4-[6-(1-methyl-1H-pyrazol-4-yl)-7H-pyrrolo[2,3-d]pyrimidin-4-yl]-benzylamide;
1,3-Dihydro-isoindole-2-carboxylic acid 2-fluoro-4-[6-(1-methyl-1H-pyrazol-4-yl)-7H-pyrrolo[2,3-d]pyrimidin-4-yl]-benzylamide;
4-tert-Butyl-N-(4-{6-[1-(2-dimethylamino-ethyl)-1H-pyrazol-4-yl]-7H-pyrrolo[2,3-d]pyrimidin-4-yl}-2-fluoro-benzyl)-benzamide;
3-tert-Butoxy-azetidine-1-carboxylic acid 4-{6-[1-(2-dimethylamino-ethyl)-1H-pyrazol-4-yl]-7H-pyrrolo[2,3-d]pyrimidin-4-yl}-2-fluoro-benzylamide;
1,3-Dihydro-isoindole-2-carboxylic acid 4-{6-[1-(2-dimethylamino-ethyl)-1H-pyrazol-4-yl]-7H-pyrrolo[2,3-d]pyrimidin-4-yl}-2-fluoro-benzylamide;
[4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid ethyl ester;
[4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid;
N-(2-fluoro-4-(6-(1-methyl-1H-pyrazol-4-yl)-7H-pyrrolo[2,3-d]pyrimidin-4-yl)benzyl)-4,5,6,7-tetrahydropyrazolo[1,5-a]pyridine-2-carboxamide;
5-tert-Butyl-isoxazole-3-carboxylicacid 2-fluoro-4-[6-(1-methyl-1H-pyrazol-4-yl)-7H-pyrrolo[2,3-d]pyrimidin-4-yl]-benzylamide;
3-tert-Butyl-[1,2,4]oxadiazole-5-carboxylic acid 2-fluoro-4-[6-(1-methyl-1H-pyrazol-4-yl)-7H-pyrrolo[2,3-d]pyrimidin-4-yl]-benzylamide;
{2-Fluoro-4-[6-(1-methyl-1H-pyrazol-4-yl)-7H-pyrrolo[2,3-d]pyrimidin-4-yl]-benzyl}-carbamic acid tert-butyl ester; and
N-{2-Fluoro-4-[6-(1-methyl-1H-pyrazol-4-yl)-7H-pyrrolo[2,3-d]pyrimidin-4-yl]-benzyl}-benzamide.

The application provides a method for treating an inflammatory and/or autoimmune condition comprising administering to a patient in need thereof a therapeutically effective amount of the compound of Formula I.

The application provides a method for treating rheumatoid arthritis comprising administering to a patient in need thereof a therapeutically effective amount of the compound of Formula I.

The application provides a method for treating asthma comprising administering to a patient in need thereof a therapeutically effective amount of the compound of Formula I.

The application provides a pharmaceutical composition comprising the compound of Formula I.

The application provides a pharmaceutical composition comprising the compound of Formula I, admixed with at least one pharmaceutically acceptable carrier, excipient or diluent.

The application provides a use of the compound of formula I in the manufacture of a medicament for the treatment of an inflammatory disorder.

The application provides a use of the compound of formula I in the manufacture of a medicament for the treatment of an autoimmune disorder.

The application provides a use of the compound of formula I in the manufacture of a medicament for the treatment of rheumatoid arthritis.

The application provides a use of the compound of formula I in the manufacture of a medicament for the treatment of asthma.

The application provides the use of a compound as described above for the treatment of inflammatory and/or autoimmune condition.

The application provides the use of a compound as described above for the treatment of rheumatoid arthritis.

The application provides the use of a compound as described above for the treatment of asthma.

The application provides a compound as described above for use in the treatment of inflammatory and/or autoimmune condition.

The application provides a compound as described above for use in the treatment of rheumatoid arthritis.

The application provides a compound as described above for use in the treatment of asthma.

The application provides a compound, method, or composition as described herein.

The application provides a method for treating an inflammatory and/or autoimmune condition comprising administering to a patient in need thereof a therapeutically effective amount of the Btk inhibitor compound of Formula I′.

The application provides a method for treating arthritis comprising administering to a patient in need thereof a therapeutically effective amount of the Btk inhibitor compound of Formula I′.

The application provides a method for treating asthma comprising administering to a patient in need thereof a therapeutically effective amount of the Btk inhibitor compound of Formula I′.

The application provides a method of inhibiting B-cell proliferation comprising administering to a patient in need thereof a therapeutically effective amount of the Btk inhibitor compound of Formula I′.

The application provides a method for inhibiting Btk activity comprising administering the Btk inhibitor compound of any one of Formula I′, wherein the Btk inhibitor compound exhibits an IC₅₀ of 50 micromolar or less in an in vitro biochemical assay of Btk activity.

In one variation of the above method, the Btk inhibitor compound exhibits an IC₅₀ of 100 nanomolar or less in an in vitro biochemical assay of Btk activity.

In another variation of the above method, the compound exhibits an IC₅₀ of 10 nanomolar or less in an in vitro biochemical assay of Btk activity.

The application provides a method for treating an inflammatory condition comprising co-administering to a patient in need thereof a therapeutically effective amount of an anti-inflammatory compound in combination with the Btk inhibitor compound of Formula I′.

The application provides a method for treating arthritis comprising co-administering to a patient in need thereof a therapeutically effective amount of an anti-inflammatory compound in combination with the Btk inhibitor compound of Formula I′.

The application provides a method for treating a lymphoma or a BCR-ABL1⁺ leukemia cells by administering to a patient in need thereof a therapeutically effective amount of the Btk inhibitor compound of Formula I′.

The application provides a pharmaceutical composition comprising the Btk inhibitor compound of Formula I′, admixed with at least one pharmaceutically acceptable carrier, excipient or diluent.

The application provides a use of the compound of formula I′ in the manufacture of a medicament for the treatment of an inflammatory disorder.

The application provides a use of the compound of formula I′ in the manufacture of a medicament for the treatment of an autoimmune disorder.

The application provides a compound, method, or composition as described herein.

### Compounds and Preparation

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formula I:

TABLE I

| Compound | Nomenclature | Structure |
|---|---|---|
| I-1 | 4-(4-Chloro-phenyl)-7H- pyrrolo[2,3-d]pyrimidine | 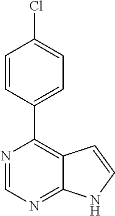 [SMILES: Clc1ccc(-c2ncnc3[nH]ccc23)cc1] |
| I-2 | 4-(3-Chloro-phenyl)-7H- pyrrolo[2,3-d]pyrimidine | 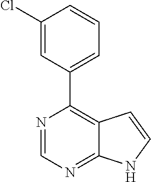 [SMILES: Clc1cccc(-c2ncnc3[nH]ccc23)c1] |
| I-3 | 4-(2-Chloro-phenyl)-7H- pyrrolo[2,3-d]pyrimidine | 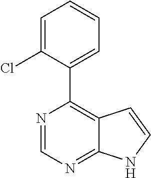 [SMILES: Clc1ccccc1-c1ncnc2[nH]ccc12] |
| I-4 | 4-(3-Fluoro-4-methyl- phenyl)-7H-pyrrolo[2,3- d]pyrimidine | 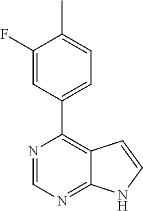 [SMILES: Cc1ccc(-c2ncnc3[nH]ccc23)cc1F] |
| I-5 | 4-(2,4-Dimethyl-phenyl)- 7H-pyrrolo[2,3- d]pyrimidine | 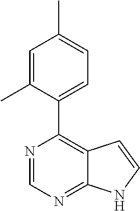 [SMILES: Cc1ccc(-c2ncnc3[nH]ccc23)c(C)c1] |
| I-6 | 4-(3,4-Dimethyl-phenyl)- 7H-pyrrolo[2,3- d]pyrimidine | 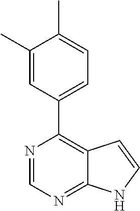 [SMILES: Cc1ccc(-c2ncnc3[nH]ccc23)cc1C] |
| I-7 | 4-p-Tolyl-7H-pyrrolo[2,3- d]pyrimidine | 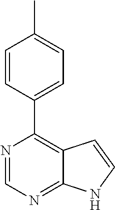 [SMILES: Cc1ccc(-c2ncnc3[nH]ccc23)cc1] |
| I-8 | 4-(3-Chloro-4-methyl- phenyl)-7H-pyrrolo[2,3- d]pyrimidine | 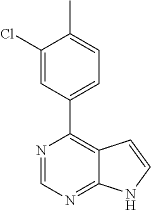 [SMILES: Cc1ccc(-c2ncnc3[nH]ccc23)cc1Cl] |
| I-9 | 4-tert-Butyl-N-[4-(7H- pyrrolo[2,3-d]pyrimidin-4- yl)-benzyl]-benzamide | 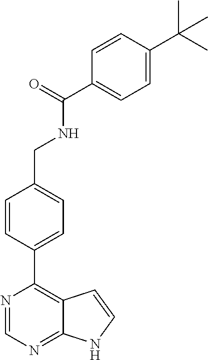 [SMILES: CC(C)(C)c1ccc(C(=O)NCc2ccc(-c3ncnc4[nH]ccc34)cc2)cc1] |
| I-10 | 3-Chloro-N-[4-(7H- pyrrolo[2,3-d]pyrimidin-4- yl)-benzyl]-benzamide | 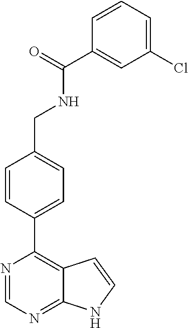 [SMILES: O=C(NCc1ccc(-c2ncnc3[nH]ccc23)cc1)c1cccc(Cl)c1] |
| I-11 | 2-(3-Chloro- phenylamino)-N-[4-(7H- pyrrolo[2,3-d]pyrimidin-4- yl)-benzyl]-acetamide | 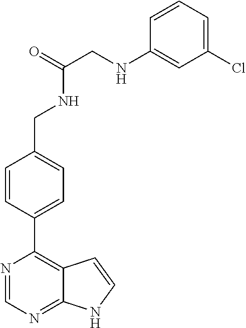 [SMILES: O=C(CNc1cccc(Cl)c1)NCc1ccc(-c2ncnc3[nH]ccc23)cc1] |
| I-12 | 4-tert-Butyl-N-[2-fluoro- 4-(7H-pyrrolo[2,3- d]pyrimidin-4-yl)-benzyl]- benzamide | 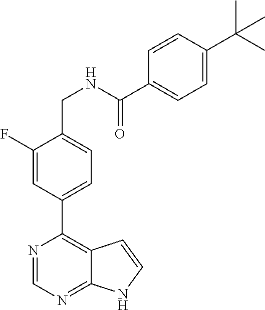 [SMILES: CC(C)(C)c1ccc(C(=O)NCc2ccc(-c3ncnc4[nH]ccc34)cc2F)cc1] |
| I-13 | 4-{4-[(4-tert-Butyl- benzoylamino)-methyl]-3- fluoro-phenyl)-7H- pyrrolo[2,3-d]pyrimidine- 6-carboxylic acid tert- butyl ester | 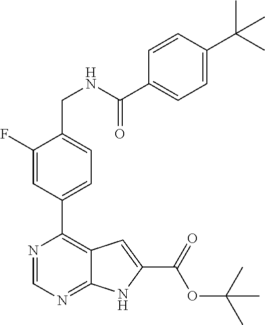 [SMILES: CC(C)(C)OC(=O)c1cc2c(-c3ccc(CNC(=O)c4ccc(C(C)(C)C)cc4)c(F)c3)ncnc2[nH]1] |
| I-14 | 4-(4-((4-tert- butylbenzamido)methyl)- 3-fluorophenyl)-7H- pyrrolo[2,3-d]pyrimidine- 6-carboxylic acid | 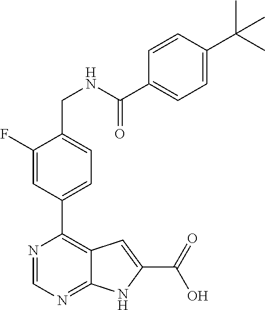 [SMILES: CC(C)(C)c1ccc(C(=O)NCc2ccc(-c3ncnc4[nH]c(C(=O)O)cc34)cc2F)cc1] |
| I-15 | 4-tert-butyl-N-(2-fluoro-4- (6-(morpholine-4- carbonyl)-7H-pyrrolo[2,3- d]pyrimidin-4- yl)benzyl)benzamide | 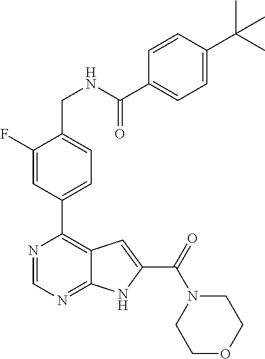 [SMILES: CC(C)(C)c1ccc(C(=O)NCc2ccc(-c3ncnc4[nH]c(C(=O)N5CCOCC5)cc34)cc2F)cc1] |
| I-16 | 4-{4-[(4-tert-Butyl- benzoylamino)-methyl]-3- fluoro-phenyl}-7H- pyrrolo[2,3-d]pyrimidine- 6-carboxylic acid dimethylamide | 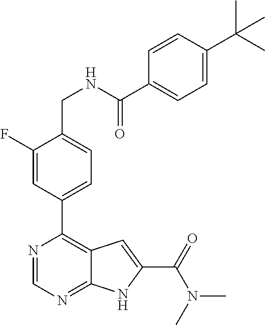 [SMILES: CN(C)C(=O)c1cc2c(-c3ccc(CNC(=O)c4ccc(C(C)(C)C)cc4)c(F)c3)ncnc2[nH]1] |
| I-17 | 4-{4-[(4-tert-Butyl- benzoylamino)-methyl]-3- fluoro-phenyl}-7H- pyrrolo[2,3-d] pyrimidine- 6-carboxylic acid methylamide | 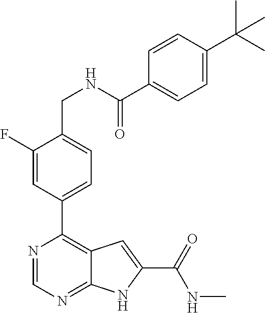 [SMILES: CNC(=O)c1cc2c(-c3ccc(CNC(=O)c4ccc(C(C)(C)C)cc4)c(F)c3)ncnc2[nH]1] |
| I-18 | 4-{4-[(4-tert-Butyl- benzoylamino)-methyl]-3- fluoro-phenyl}-7H- pyrrolo[2,3-d] pyrimidine- 6-carboxylic acid (2- hydroxy-ethyl)-amide | 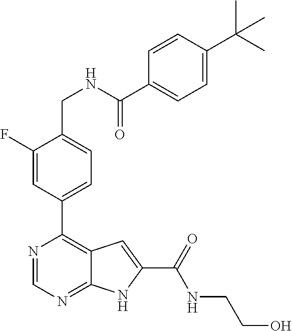 [SMILES: CC(C)(C)c1ccc(C(=O)NCc2ccc(-c3ncnc4[nH]c(C(=O)NCCO)cc34)cc2F)cc1] |
| I-19 | 4-{4-[(4-tert-Butyl- benzoylamino)-methyl]-3- fluoro-phenyl}-7H- pyrrolo[2,3-d] pyrimidine- 6-carboxylic acid (2- dimethylamino-ethyl)- amide | 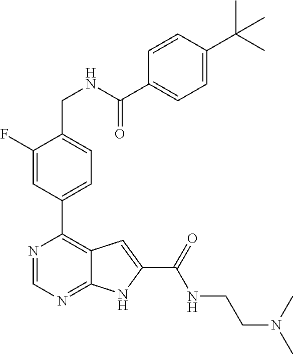 [SMILES: CN(C)CCNC(=O)c1cc2c(-c3ccc(CNC(=O)c4ccc(C(C)(C)C)cc4)c(F)c3)ncnc2[nH]1] |
| I-20 | 4-tert-Butyl-N-{1-[6-(1- methyl-1H-pyrazol-4-yl)- 7H-pyrrolo[2,3- d]pyrimidin-4-yl]- piperidin-4-ylmethyl}- benzamide | 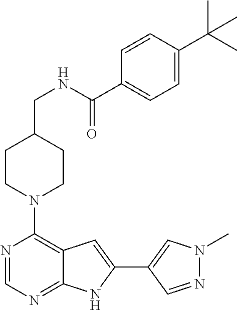 [SMILES: Cn1cc(-c2cc3c(N4CCC(CNC(=O)c5ccc(C(C)(C)C)cc5)CC4)ncnc3[nH]2)cn1] |
| I-21 | 4-tert-Butyl-N-{4-[6-(1- methyl-1H-pyrazol-4-yl)- 7H-pyrrolo[2,3- d]pyrimidin-4-yl]- benzyl}-benzamide | 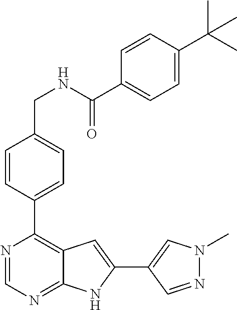 [SMILES: Cn1cc(-c2cc3c(-c4ccc(CNC(=O)c5ccc(C(C)(C)C)cc5)cc4)ncnc3[nH]2)cn1] |
| I-22 | 4-Cyclopropyl-N-{4-[6- (1-methyl-1H-pyrazol-4- yl)-7H-pyrrolo[2,3- d]pyrimidin-4-yl]- benzyl}-benzamide | 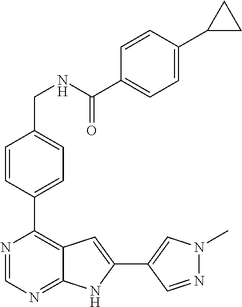 [SMILES: Cn1cc(-c2cc3c(-c4ccc(CNC(=O)c5ccc(C6CC6)cc5)cc4)ncnc3[nH]2)cn1] |
| I-23 | 4-Isopropyl-N-{4-[6-(1- methyl-1H-pyrazol-4-yl)- 7H-pyrrolo[2,3- d]pyrimidin-4-yl]- benzyl}-benzamide | 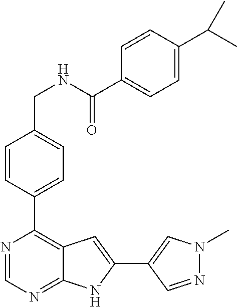 [SMILES: CC(C)c1ccc(C(=O)NCc2ccc(-c3ncnc4[nH]c(-c5cnn(C)c5)cc34)cc2)cc1] |
| I-24 | N-{4-[6-(1-Methyl-1H- pyrazol-4-yl)-7H- pyrrolo[2,3-d]pyrimidin-4- yl]-benzy]}-4-oxetan-3-yl- benzamide | 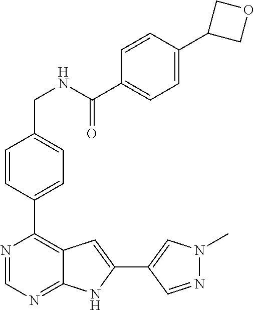 [SMILES: Cn1cc(-c2cc3c(-c4ccc(CNC(=O)c5ccc(C6COC6)cc5)cc4)ncnc3[nH]2)cn1] |
| I-25 | 4-(3-Methyl-oxetan-3-yl)- N-{4-[6-(1-methyl-1H- pyrazol-4-yl)-7H- pyrrolo[2,3-d]pyrimidin-4- yl]-benzyl}-benzamide | 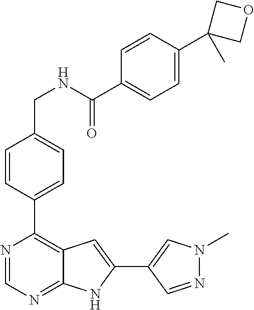 [SMILES: Cn1cc(-c2cc3c(-c4ccc(CNC(=O)c5ccc(C6(C)COC6)cc5)cc4)ncnc3[nH]2)cn1] |
| I-26 | 4,5,6,7-Tetrahydro- benzo[b]thiophene-2- carboxylic acid 4-[6-(1- methyl-1H-pyrazol-4-yl)- 7H-pyrrolo[2,3- d]pyrimidin-4-yl]- benzylamide | 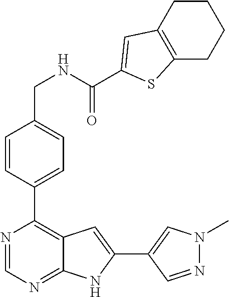 [SMILES: Cn1cc(-c2cc3c(-c4ccc(CNC(=O)c5cc6c(s5)CCCC6)cc4)ncnc3[nH]2)cn1] |
| I-27 | 4-tert-Butyl-N-{2-fluoro- 4-[6-(1-methyl-1H- pyrazol-4-yl)-7H- pyrrolo[2,3-d]pyrimidin-4- yl]-benzyl}-benzamide | 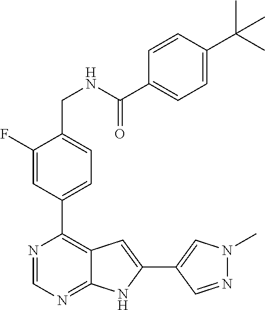 [SMILES: Cn1cc(-c2cc3c(-c4ccc(CNC(=O)c5ccc(C(C)(C)C)cc5)c(F)c4)ncnc3[nH]2)cn1] |
| I-28 | 6-tert-Butyl-N-{2-fluoro- 4-[6-(1-methyl-1H- pyrazol-4-yl)-7H- pyrrolo[2,3-d]pyrimidin-4- yl]-benzyl}-nicotinamide | 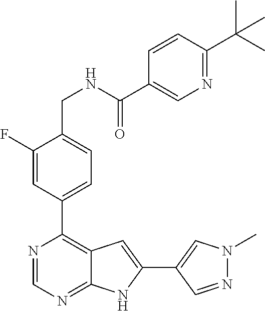 [SMILES: Cn1cc(-c2cc3c(-c4ccc(CNC(=O)c5ccc(C(C)(C)C)nc5)c(F)c4)ncnc3[nH]2)cn1] |
| I-29 | 5-Methyl-thiophene-2- carboxylic acid 2-fluoro-4- [6-(1-methyl-1H-pyrazol- 4-yl)-7H-pyrrolo[2,3-d] pyrimidin-4-yl]- benzylamide | 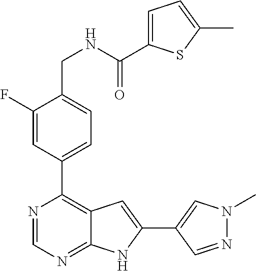 [SMILES: Cc1ccc(C(=O)NCc2ccc(-c3ncnc4[nH]c(-c5cnn(C)c5)cc34)cc2F)s1] |
| I-30 | 4-tert-Butyl-N-(2-fluoro- 4-{6-[1-(2-hydroxy-ethyl)- 1H-pyrazol-4-yl]-7H- pyrrolo[2,3-d]pyrimidin-4- yl}-benzyl)-benzamide | 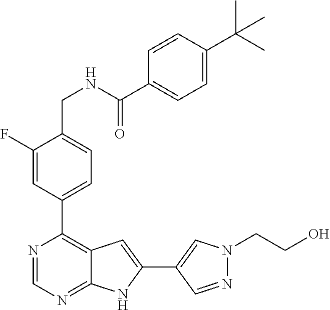 [SMILES: CC(C)(C)c1ccc(C(=O)NCc2ccc(-c3ncnc4[nH]c(-c5cnn(CCO)c5)cc34)cc2F)cc1] |
| I-31 | 4-tert-Butyl-N-{2-fluoro- 4-[6-(1-methyl-1H- pyrazol-4-yl)-7H- pyrrolo[2,3-d]pyrimidin-4- yl]-benzyl}-N-methyl- benzamide | 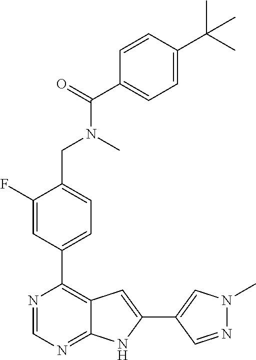 [SMILES: CN(Cc1ccc(-c2ncnc3[nH]c(-c4cnn(C)c4)cc23)cc1F)C(=O)c1ccc(C(C)(C)C)cc1] |
| I-32 | 5-Methyl-thiophene-2- carboxylic acid {2-fluoro- 4-[6-(1-methyl-1H- pyrazol-4-yl)-7H- pyrrolo[2,3-d] pyrimidin- 4-yl]-benzyl}-methyl- amide | 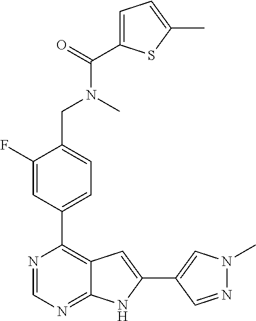 [SMILES: Cc1ccc(C(=O)N(C)Cc2ccc(-c3ncnc4[nH]c(-c5cnn(C)c5)cc34)cc2F)s1] |
| I-33 | 2-tert-Butyl-5-{2-fluoro-4- [6-(1-methyl-1H-pyrazol- 4-yl)-7H-pyrrolo[2,3- d]pyrimidin-4-yl]- benzyl}-4,5-dihydro- thieno[2,3-c]pyrrol-6-one | 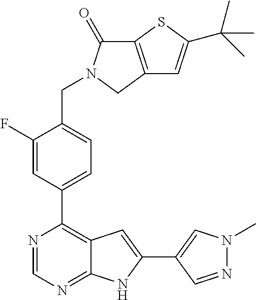 [SMILES: Cn1cc(-c2cc3c(-c4ccc(CN5Cc6cc(C(C)(C)C)sc6C5=O)c(F)c4)ncnc3[nH]2)cn1] |
| I-34 | 5-tert-Butyl-isoxazole-3- carboxylic acid 2-fluoro-4- [6-(1-methyl-1H-pyrazol- 4-yl)-7H-pyrrolo[2,3- d]pyrimidin-4-yl]- benzylamide | 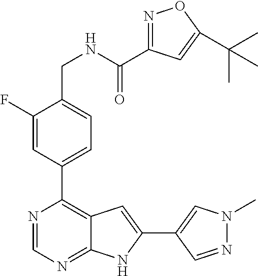 [SMILES: Cn1cc(-c2cc3c(-c4ccc(CNC(=O)c5cc(C(C)(C)C)on5)c(F)c4)ncnc3[nH]2)cn1] |
| I-35 | N-{2-Fluoro-4-[6-(1- methyl-1H-pyrazol-4-yl)- 7H-pyrrolo[2,3- d]pyrimidin-4-yl]- benzyl}-4-(3-methyl- oxetan-3-yl)-benzamide | 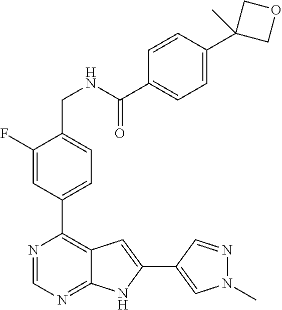 [SMILES: Cn1cc(-c2cc3c(-c4ccc(CNC(=O)c5ccc(C6(C)COC6)cc5)c(F)c4)ncnc3[nH]2)cn1] |
| I-36 | 4-(Cyano-dimethyl- methyl)-N-{2-fluoro-4-[6- (1-methyl-1H-pyrazol-4- yl)-7H-pyrrolo[2,3- d]pyrimidin-4-yl]- benzyl}-benzamide | 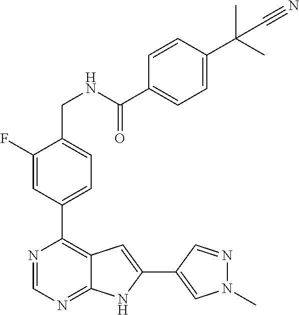 [SMILES: Cn1cc(-c2cc3c(-c4ccc(CNC(=O)c5ccc(C(C)(C)C#N)cc5)c(F)c4)ncnc3[nH]2)cn1] |
| I-37 | 4,5,6,7-Tetrahydro- benzo[b]thiophene-2- carboxylic acid 2-fluoro-4- [6-(1-methyl-1H-pyrazol- 4-yl)-7H-pyrrolo[2,3- d]pyrimidin-4-yl]- benzylamide | 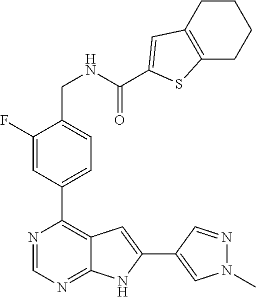 [SMILES: Cn1cc(-c2cc3c(-c4ccc(CNC(=O)c5cc6c(s5)CCCC6)c(F)c4)ncnc3[nH]2)cn1] |
| I-38 | N-{2-Fluoro-4-[6-(1- methyl-1H-pyrazol-4-yl)- 7H-pyrrolo[2,3- d]pyrimidin-4-yl]- benzyl}-4-(1-hydroxy-1- methyl-ethyl)-benzamide | 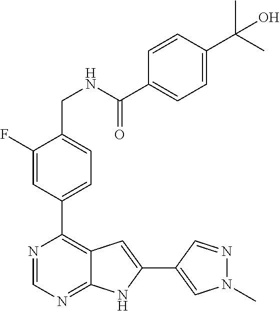 [SMILES: Cn1cc(-c2cc3c(-c4ccc(CNC(=O)c5ccc(C(C)(C)O)cc5)c(F)c4)ncnc3[nH]2)cn1] |
| I-39 | 3-tert-Butyl-isoxazole-5- carboxylic acid 2-fluoro-4- [6-(1-methyl-1H-pyrazol- 4-yl)-7H-pyrrolo[2,3- d]pyrimidin-4-yl]-benzyl amide | 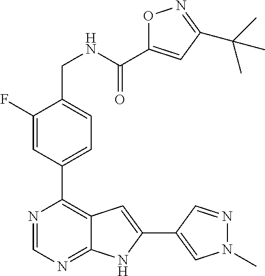 [SMILES: Cn1cc(-c2cc3c(-c4ccc(CNC(=O)c5cc(C(C)(C)C)no5)c(F)c4)ncnc3[nH]2)cn1] |
| I-40 | 3-tert-Butoxy-azetidine-1- carboxylic acid 2-fluoro-4- [6-(1-methyl-1H-pyrazol- 4-yl)-7H-pyrrolo[2,3- d]pyrimidin-4-yl]- benzylamide | 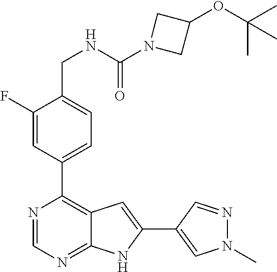 [SMILES: Cn1cc(-c2cc3c(-c4ccc(CNC(=O)N5CC(OC(C)(C)C)C5)c(F)c4)ncnc3[nH]2)cn1] |
| I-41 | 1,3-Dihydro-isoindole-2- carboxylic acid 2-fluoro-4- [6-(1-methyl-1H-pyrazol- 4-yl)-7H-pyrrolo[2,3- d]pyrimidin-4-yl]- benzylamide | 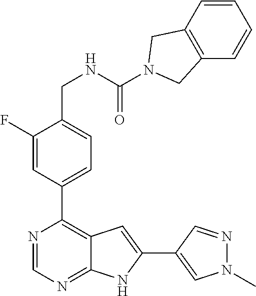 [SMILES: Cn1cc(-c2cc3c(-c4ccc(CNC(=O)N5Cc6ccccc6C5)c(F)c4)ncnc3[nH]2)cn1] |
| I-42 | 4-tert-Butyl-N-(4-{6-[1- (2-dimethylamino-ethyl)- 1H-pyrazol-4-yl]-7H- pyrrolo[2,3-d]pyrimidin-4- yl}-2-fluoro-benzyl)- benzamide | 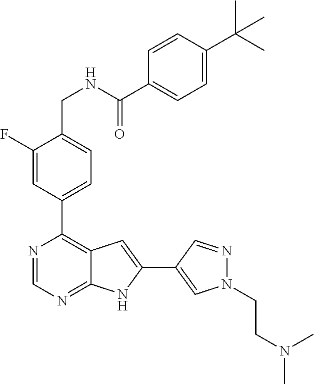 [SMILES: CN(C)CCn1cc(-c2cc3c(-c4ccc(CNC(=O)c5ccc(C(C)(C)C)cc5)c(F)c4)ncnc3[nH]2)cn1] |
| I-43 | 3-tert-Butoxy-azetidine-1- carboxylic acid 4-{6-[1- (2-dimethylamino-ethyl)- 1H-pyrazol-4-yl]-7H- pyrrolo[2,3-d]pyrimidin-4- yl}-2-fluoro-benzylamide | 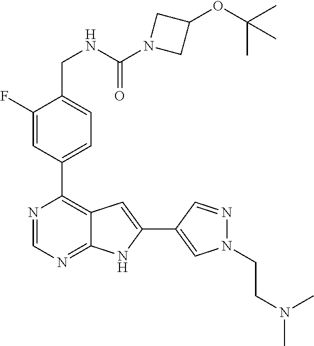 [SMILES: CN(C)CCn1cc(-c2cc3c(-c4ccc(CNC(=O)N5CC(OC(C)(C)C)C5)c(F)c4)ncnc3[nH]2)cn1] |
| I-44 | 1,3-Dihydro-isoindole-2- carboxylic acid 4-{6-[1- (2-dimethylamino-ethyl)- 1H-pyrazol-4-yl]-7H- pyrrolo[2,3-d]pyrimidin-4- yl}-2-fluoro-benzylamide | 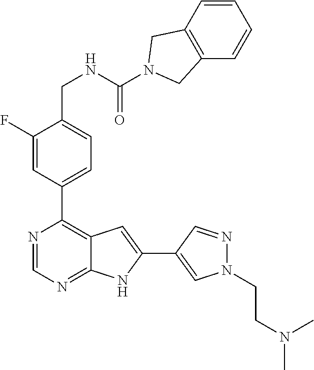 [SMILES: CN(C)CCn1cc(-c2cc3c(-c4ccc(CNC(=O)N5Cc6ccccc6C5)c(F)c4)ncnc3[nH]2)cn1] |
| I-45 | [4-(4-{4-[(4-tert-Butyl- benzoylamino)-methyl]-3- fluoro-phenyl}-7H- pyrrolo[2,3-d]pyrimidin-6- yl)-pyrazol-1-yl]-acetic acid ethyl ester | 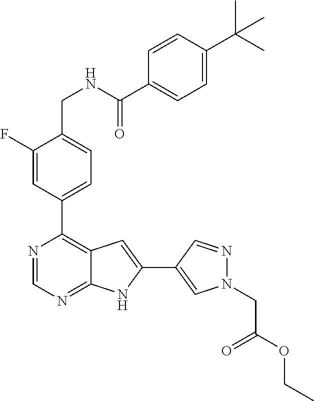 [SMILES: CCOC(=O)Cn1cc(-c2cc3c(-c4ccc(CNC(=O)c5ccc(C(C)(C)C)cc5)c(F)c4)ncnc3[nH]2)cn1] |
| I-46 | [4-(4-{4-[(4-tert-Butyl- benzoylamino)-methyl]-3- fluoro-phenyl}-7H- pyrrolo[2,3-d]pyrimidin-6- yl)-pyrazol-1-yl]-acetic acid | 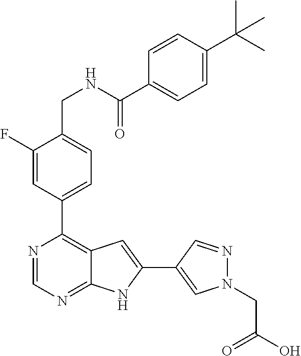 [SMILES: CC(C)(C)c1ccc(C(=O)NCc2ccc(-c3ncnc4[nH]c(-c5cnn(CC(=O)O)c5)cc34)cc2F)cc1] |
| I-47 | N-(2-fluoro-4-(6-(1- methyl-1H-pyrazol-4-yl)- 7H-pyrrolo[2,3- d]pyrimidin-4-yl)benzyl)- 4,5,6,7- tetrahydropyrazolo[1,5- a]pyridine-2-carboxamide | 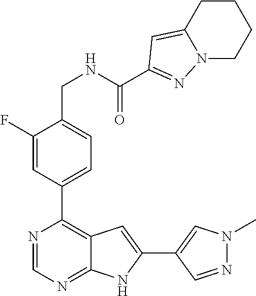 [SMILES: Cn1cc(-c2cc3c(-c4ccc(CNC(=O)c5cc6n(n5)CCCC6)c(F)c4)ncnc3[nH]2)cn1] |
| I-48 | 5-tert-Butyl-isoxazole-3- carboxylicacid 2-fluoro-4- [6-(1-methyl-1H-pyrazol- 4-yl)-7H-pyrrolo[2,3- d]pyrimidin-4-yl]- benzylamide | 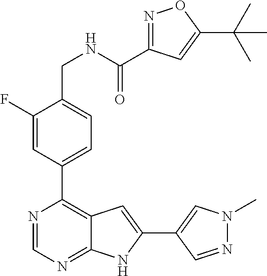 [SMILES: Cn1cc(-c2cc3c(-c4ccc(CNC(=O)c5cc(C(C)(C)C)on5)c(F)c4)ncnc3[nH]2)cn1] |
| I-49 | 3-tert-Butyl- [1,2,4]oxadiazole-5- carboxylic acid 2-fluoro-4- [6-(1-methyl-1H-pyrazol- 4-yl)-7H-pyrrolo[2,3- d]pyrimidin-4-yl]- benzylamide | 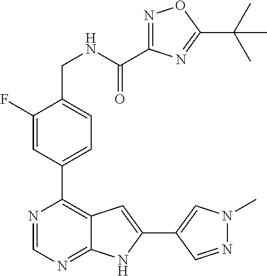 [SMILES: Cn1cc(-c2cc3c(-c4ccc(CNC(=O)c5noc(C(C)(C)C)n5)c(F)c4)ncnc3[nH]2)cn1] |
| I-50 | {2-Fluoro-4-[6-(1-methyl- 1H-pyrazol-4-yl)-7H- pyrrolo[2,3-d]pyrimidin-4- yl]-benzyl}-carbamic acid tert-butyl ester | 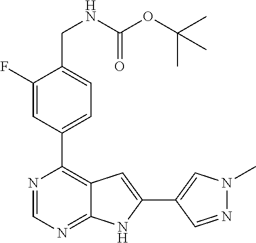 [SMILES: Cn1cc(-c2cc3c(-c4ccc(CNC(=O)OC(C)(C)C)c(F)c4)ncnc3[nH]2)cn1] |
| I-51 | N-{2-Fluoro-4-[6-(1- methyl-1H-pyrazol-4-yl)- 7H-pyrrolo[2,3- d]pyrimidin-4-yl]- benzyl}-benzamide | 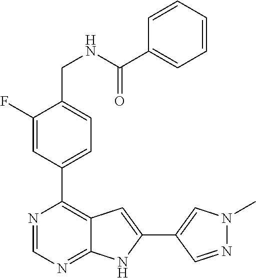 [SMILES: Cn1cc(-c2cc3c(-c4ccc(CNC(=O)c5ccccc5)c(F)c4)ncnc3[nH]2)cn1] |

### General Synthetic Schemes

The compounds of the present invention may be prepared by processes known in the art. Suitable processes for synthesizing these compounds are provided in the examples. Generally, compounds of the invention may be prepared according to one of the below described synthetic routes (Schemes 1-5). The starting materials are either commercially available or can be synthesized by methods known to those of ordinary skill in the art.

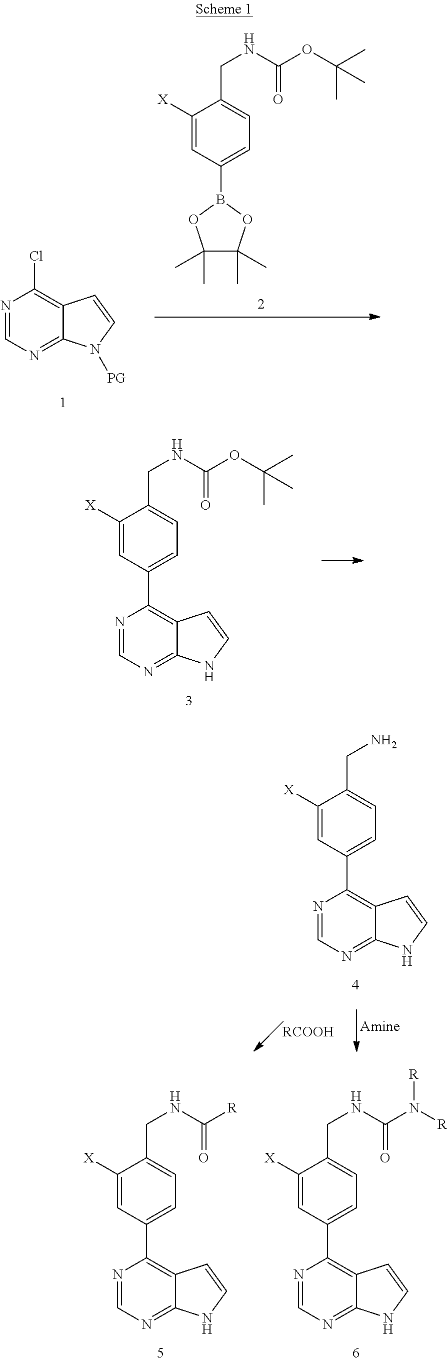

Compounds of interest of formula 5 and 6, where X is either fluorine, hydrogen or methyl and R is as described above in the genus of formula I, can be prepared according to scheme 1. Starting from commercially available 4-chloro-7-tosyl-7H-pyrrolo[2,3-d]pyrimidine 1, a Suzuki aryl-aryl coupling reaction with boronate ester 2 provides derivative 3. The conditions for the Suzuki aryl-aryl coupling have been reviewed in Modern Arene Chemistry 2002, 53-106. In carrying out this reaction any of the conditions conventional in a Suzuki reaction can be utilized. Generally Suzuki coupling reactions are carried out in the presence of a transition metal catalyst such as tetrakis(triphenylphosphine)palladium(0)), a conventional organic solvent such as dimethoxyethane and a weak inorganic base such as potassium carbonate. The reaction is carried out at a temperature between room temperature and about 100° C. for reaction times between 1 hour and several hours, if using conventional heating. The reaction can be also effected by microwave irradiation which is usually carried out at higher temperatures (for example 160° C.) but shorter time (5-60 min). During the reaction, loss of the tosyl group is also observed. The tert-butoxycarbonyl (BOC) protecting group in derivative 3 could easily be removed under acidic conditions such as a mixture of trifluoroacetic acid (TFA) and dichloromethane (DCM) to generate the free amine derivative 4. The reaction can occur at room temperature for reaction times between 15 minutes to 3 hours. Coupling reaction between 4 and carboxylic acid derivatives can be accomplished using standard peptidic coupling reagents such as 0-(7-azabenzotriazol-1-yl)-N,N,N′,N′-tetramethyluronium hexafluoro-phosphate (HATU), a conventional organic solvent such as N,N-dimethylformamide (DMF) and a base such as diisopropyl ethyl amine (DIPEA) to afford compound such as 5. A list of coupling reagents that could also be used for this transformation can be found in this review (Chemical Review 2011, 111, 6557). The reaction can occur at room temperature for reaction times between one hour to several hours. Alternatively, the free amine 4 can be coupled to other amine using 1,1′-carbonyl diimidazole as coupling reagent to prepare urea derivatives such as 6. The reaction can occur using DMF at temperature between room temperature and 90° C. for several hours. As known by those skilled in the art, other protecting groups than a tosyl group or BOC group could be used for this scheme. (For a leading reference, see P.G.M. Wuts and T. W. Greene in Green's Protective Groups in Organic Synthesis, Wiley and Sons, 2007).

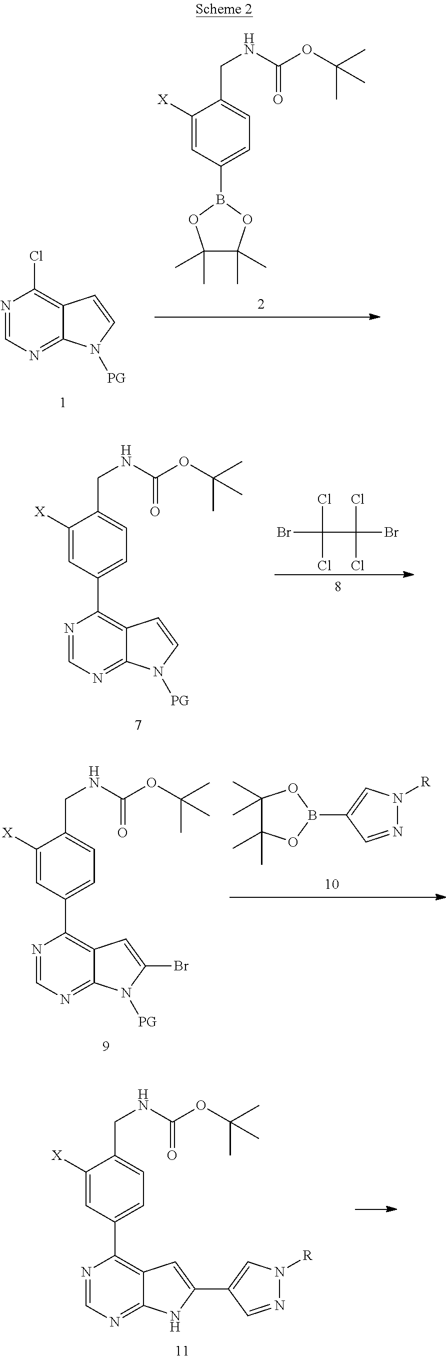

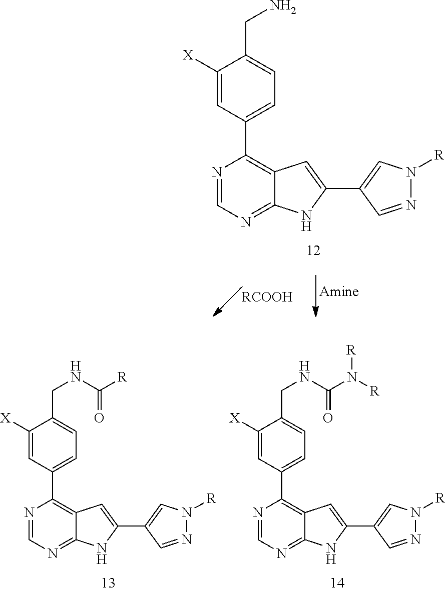

Compounds of interest of formula 13 and 14, where X is either fluorine, hydrogen or methyl and R is as described above in the genus of formula I, can be prepared according to scheme 2. The Suzuki aryl-aryl coupling, as described in scheme 1 was accomplished. However, using a shorter reaction time (30 minutes) and heating under microwave irradiation at 160° C., the tosyl protecting group could be maintained under those conditions. The bromination at C-2 of the pyrrolopyrimidine scaffold can be achieved using 1,2-dibromo-tetrachloroethane in presence of a strong base such as lithium diisopropyl amide (LDA) to afford derivative 9. The reaction can occur in inert solvent such as tetrahydrofuran (THF) at −78° C. for reaction times between 2 hours and several hours (WO2004/093812) The Suzuki coupling between 9 and 10 can occur using standard Suzuki conditions. The reaction is using longer reaction times (60 minutes) under microwave irradiation at 160° C. and in this case the tosyl protecting group is also removed. The subsequent steps to prepare derivatives 13 and 14 have been described above.

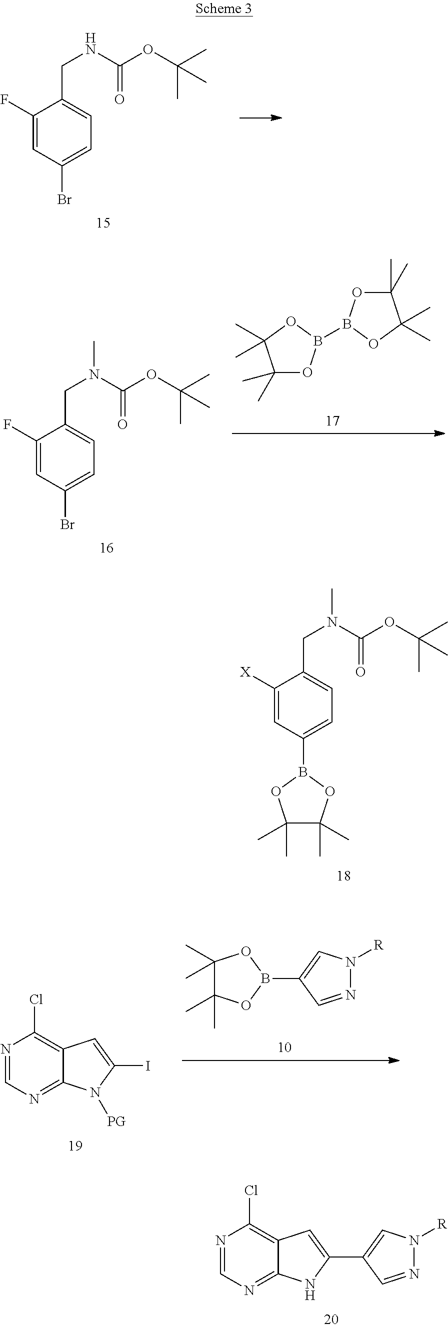

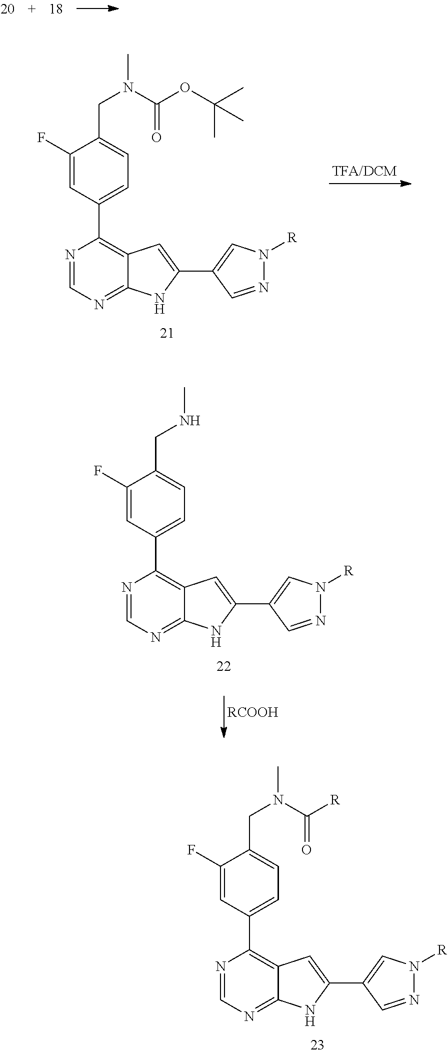

The scheme 3 describes the synthesis of compound such as 23. Methylation on the nitrogen of the carbamate 15 can occur using a strong base such as sodium hydride (NaH) in presence of methyl iodide and a polar solvent such as DMF. The reaction proceeds at 4° C. to room temperature and for reaction times between 2 hours to several hours. The palladium catalyzed borylation reaction of carbamate 16 can occur using bis(pinacoloto)diboron 17, a suitable palladium catalyst source such as 1,1′-bis(diphenylphosphino)ferrocene-palladium(II)dichloride, and potassium acetate (Journal of Organic Chemistry 1995, 60, 7508-7510). The reaction may proceed in an appropriate solvent such as dioxane, DMF, or NMP using either conventional heating or microwave heating at temperatures between 90° C. and 150° C. for reaction times between one hour and several hours. 4-chloro-6-iodo-7-(phenylsulfonyl)-7H-pyrrolo[2,3-d]pyrimidine 19 can be coupled with boronate ester 10 using the Suzuki coupling conditions described above to provide derivative 20. Similarly, the coupling of 18 and 20 occur using the same standard conditions to afford 21. The subsequent steps to prepare derivatives 23 have been described above.

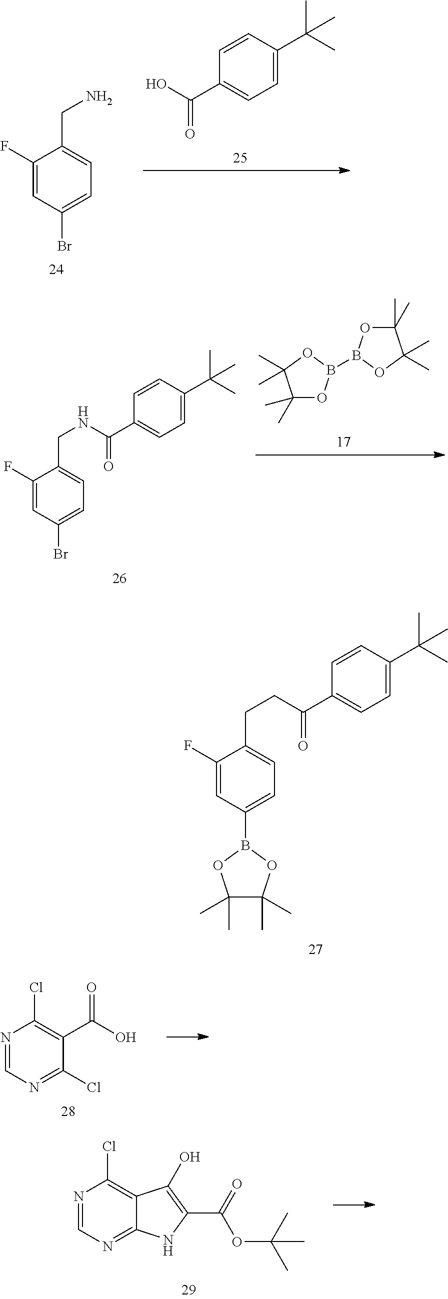

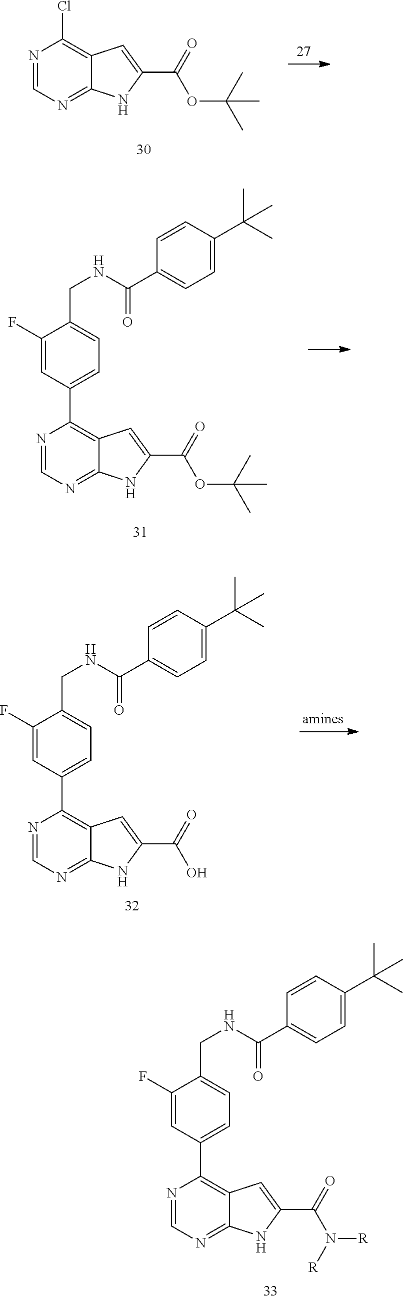

Compounds of interest of general formula 33, where R are as defined for the genus of formula I, can be prepared according to scheme 4. Coupling partner 27 can be prepared in two steps from commercially available starting materials. As described above, the formation of the amide bond in derivative 26 can be accomplished using standard coupling reagents. Similarly, the introduction of the boronate ester functionality in compound 27 can also be introduced using standard conditions. The synthesis of derivative 30 has been already described in the literature (WO2011/149827). The Suzuki coupling between 27 and 30 can occur in polar solvents such as DME, dioxane or DMF at temperature between 60° C. and 100° C. using conventional heating methods for reaction times between 1 hour to several hours. Microwave heating can reduce considerably the reaction times for the Suzuki couplings (Current Organic Chemistry, 2010, 14, 1050-1074). Usually, only 10-60 minutes are required to complete the reaction. Deprotection of the BOC group in compound 31 and subsequent coupling reaction to form the amide bonds as in 33 have been described above.

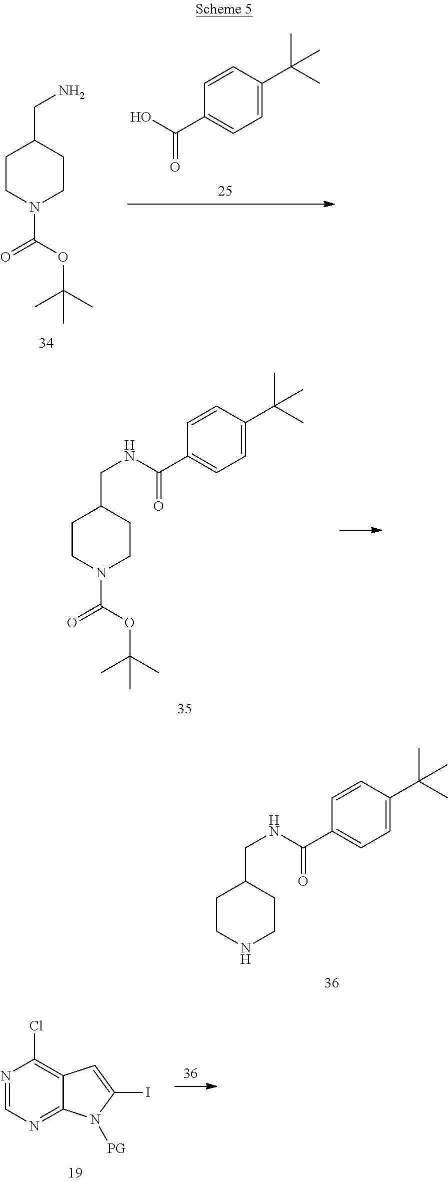

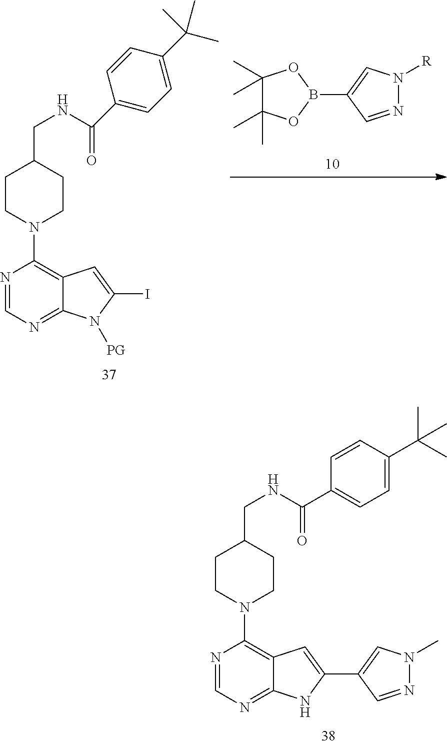

Compounds of interest such as 38 can be prepared according to scheme 5. The reaction between 19 and 36 can be accomplished in polar protic solvents such as ethanol in presence of a base such as DIPEA or triethylamine (TEA). The reaction can occur at 80° C. for reaction times between 1 hour and several hours. The subsequent steps leading to the preparation of compound 38 have been described above.

### Pharmaceutical Compositions and Administration

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Indications and Methods of Treatment

The compounds of generic Formula I inhibit Bruton's tyrosine kinase (Btk). Activation of Btk by upstream kinases results in activation of phospholipase-Cγ which, in turn, stimulates release of pro-inflammatory mediators. Compounds of Formula I are useful in the treatment of arthritis and other anti-inflammatory and auto-immune diseases. Compounds according to Formula I are, accordingly, useful for the treatment of arthritis. Compounds of Formula I are useful for inhibiting Btk in cells and for modulating B-cell development. The present invention further comprises pharmaceutical compositions containing compounds of Formula I admixed with pharmaceutically acceptable carrier, excipients or diluents.

The compounds described herein are kinase inhibitors, in particular Btk inhibitors. These inhibitors can be useful for treating one or more diseases responsive to kinase inhibition, including diseases responsive to Btk inhibition and/or inhibition of B-cell proliferation, in mammals. Without wishing to be bound to any particular theory, it is believed that the interaction of the compounds of the invention with Btk results in the inhibition of Btk activity and thus in the pharmaceutical utility of these compounds. Accordingly, the invention includes a method of treating a mammal, for instance a human, having a disease responsive to inhibition of Btk activity, and/or inhibiting B-cell proliferation, comprising administrating to the mammal having such a disease, an effective amount of at least one chemical entity provided herein. An effective concentration may be ascertained experimentally, for example by assaying blood concentration of the compound, or theoretically, by calculating bioavailability. Other kinases that may be affected in addition to Btk include, but are not limited to, other tyrosine kinases and serine/threonine kinases.

Kinases play notable roles in signaling pathways controlling fundamental cellular processes such as proliferation, differentiation, and death (apoptosis). Abnormal kinase activity has been implicated in a wide range of diseases, including multiple cancers, autoimmune and/or inflammatory diseases, and acute inflammatory reactions. The multifaceted role of kinases in key cell signaling pathways provides a significant opportunity to identify novel drugs targeting kinases and signaling pathways.

An embodiment includes a method of treating a patient having an autoimmune and/or inflammatory disease, or an acute inflammatory reaction responsive to inhibition of Btk activity and/or B-cell proliferation.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

Included herein are methods of treatment in which at least one chemical entity provided herein is administered in combination with an anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate.

Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

Still other embodiments of the invention pertain to combinations in which at least one active agent is an immunosuppressant compound such as an immunosuppressant compound chosen from methotrexate, leflunomide, cyclosporine, tacrolimus, azathioprine, and mycophenolate mofetil.

B-cells and B-cell precursors expressing BTK have been implicated in the pathology of B-cell malignancies, including, but not limited to, B-cell lymphoma, lymphoma (including Hodgkin's and non-Hodgkin's lymphoma), hairy cell lymphoma, multiple myeloma, chronic and acute myelogenous leukemia and chronic and acute lymphocytic leukemia.

BTK has been shown to be an inhibitor of the Fas/APO-1 (CD-95) death inducing signaling complex (DISC) in B-lineage lymphoid cells. The fate of leukemia/lymphoma cells may reside in the balance between the opposing proapoptotic effects of caspases activated by DISC and an upstream anti-apoptotic regulatory mechanism involving BTK and/or its substrates (Vassilev et al., J. Biol. Chem. 1998, 274, 1646-1656).

It has also been discovered that BTK inhibitors are useful as chemosensitizing agents, and, thus, are useful in combination with other chemotherapeutic drugs, in particular, drugs that induce apoptosis. Examples of other chemotherapeutic drugs that can be used in combination with chemosensitizing BTK inhibitors include topoisomerase I inhibitors (camptothecin or topotecan), topoisomerase II inhibitors (e.g. daunomycin and etoposide), alkylating agents (e.g. cyclophosphamide, melphalan and BCNU), tubulin directed agents (e.g. taxol and vinblastine), and biological agents (e.g. antibodies such as anti CD20 antibody, IDEC 8, immunotoxins, and cytokines).

Btk activity has also been associated with some leukemias expressing the bcr-abl fusion gene resulting from translocation of parts of chromosome 9 and 22. This abnormality is commonly observed in chronic myelogenous leukemia. Btk is constitutively phosphorylated by the bcr-abl kinase which initiates downstream survival signals which circumvents apoptosis in bcr-abl cells. (N. Feldhahn et al. J. Exp. Med. 2005 201(11):1837-1852).

### Methods of Treatment

The application provides a method for treating an inflammatory and/or autoimmune condition comprising administering to a patient in need thereof a therapeutically effective amount of the compound of Formula I.

The application provides a method for treating an inflammatory condition comprising administering to a patient in need thereof a therapeutically effective amount of the compound of Formula I.

The application provides a method for treating rheumatoid arthritis comprising administering to a patient in need thereof a therapeutically effective amount of the compound of Formula I.

The application provides a method for treating asthma comprising administering to a patient in need thereof a therapeutically effective amount of Formula I.

The application provides a method for treating an inflammatory and/or autoimmune condition comprising administering to a patient in need thereof a therapeutically effective amount of the Btk inhibitor compound of Formulae I.

The application provides a method for treating arthritis comprising administering to a patient in need thereof a therapeutically effective amount of the Btk inhibitor compound of Formula I.

The application provides a method for treating asthma comprising administering to a patient in need thereof a therapeutically effective amount of the Btk inhibitor compound of Formula I.

The application provides a method of inhibiting B-cell proliferation comprising administering to a patient in need thereof a therapeutically effective amount of the Btk inhibitor compound of Formula I.

The application provides a method for inhibiting Btk activity comprising administering the Btk inhibitor compound of any one of Formula I, wherein the Btk inhibitor compound exhibits an IC₅₀ of 50 micromolar or less in an in vitro biochemical assay of Btk activity.

In one variation of the above method, the Btk inhibitor compound exhibits an IC₅₀ of 100 nanomolar or less in an in vitro biochemical assay of Btk activity.

In another variation of the above method, the compound exhibits an IC₅₀ of 10 nanomolar or less in an in vitro biochemical assay of Btk activity.

The application provides a method for treating an inflammatory condition comprising co-administering to a patient in need thereof a therapeutically effective amount of an anti-inflammatory compound in combination with the Btk inhibitor compound of Formula I.

The application provides a method for treating arthritis comprising co-administering to a patient in need thereof a therapeutically effective amount of an anti-inflammatory compound in combination with the Btk inhibitor compound of Formula I.

The application provides a method for treating a lymphoma or a BCR-ABL1⁺ leukemia cells by administering to a patient in need thereof a therapeutically effective amount of the Btk inhibitor compound of Formula I.

### EXAMPLES

### General Conditions

Compounds of the present invention can be prepared beginning with the commercially available starting materials by utilizing general synthetic techniques and procedures known to those skilled in the art. Outlines below are reaction schemes suitable for preparing such compounds. Further exemplification can be found in the specific examples.

### Preparative Examples

### Specific Abbreviations

boc tert-butoxycarbonyl
CDI 1,1-carbonyldiimidazole
CH₂Cl₂ Dichloromethane
Cs₂CO₃ cesium carbonate
DCM Dichloromethane
DME Dimethoxyethane
DMF N,N-dimethylformamide
DIPEA N,N-diisopropylethylamine
DMSO Dimethylsulfoxide
EtOAc ethyl acetate
EtOH Ethanol
HATU O-(7-azabenzotriazol-1-yl)-N,N,N′,N′tetramethyluroniumhexafluorophosphate
HBTU O-(Benzotriazol-1-yl)-N,N,N′,N′-tetramethyluronium hexafluorophosphate
HCl hydrogen chloride
H₂O Water
KOH potassium hydroxide
LC-MS liquid chromatography mass spectrometry
LDA lithium diisopropylamide
LiOH lithium hydroxide
HPLC high pressure liquid chromatography
MeOH methyl alcohol
min Minutes
MgSO₄ magnesium sulfate
MW Microwave
nBuLi n-butyl lithium
NaCl sodium chloride
Na₂CO₃ sodium carbonate
NaH sodium hydride
NaHMDS sodium hexamethyldisilazane
NaOH sodium hydroxide
NaOMe sodium methoxide
Na₂SO₄ sodium sulfate
NH₄Cl ammonium chloride
NH₄OH ammonium hydroxide
NMP 1-methyl-2-pyrrolidinone
NMR nuclear magnetic resonance
Pd/C palladium on charcoal
PdCl₂(dppf) [1,1′-Bis(diphenylphosphino)ferrocene]dichloropalladium(II)
Pd(OAc)₂ palladium(II) acetate
Pd(PPh₃)₄ palladium tetrakistriphenylphosphine
PG protecting group
RT (or rt) room temperature
TEA Triethylamine
TFA trifluoroacetic acid
THF Tetrahydrofuran
TLC thin layer chromatography

### General Experimental Details

Reagents were purchased from Aldrich, Oakwood, Matrix or other suppliers and used without further purification. Reactions using microwave irradiation for heating were conducted using either a Personal Chemistry Emrys Optimizer System or a CEM Discovery System. The purification of multi-milligram to multi-gram scale was conducted by methods known know to those skilled in the art such as elution of silica gel flash column; preparative flash column purifications were also effected in some cases by use of disposal pre-packed multigram silica gel columns (RediSep) eluted with a CombiFlash system. Biotage™ and ISCO™ are also flash column instruments that may have been used in this invention for purification of intermediates.

For the purpose of judging compound identity and purity, LC/MS (liquid chromatography/mass spectroscopy) spectra were recorded using the following system. For measurement of mass spectra, the system consists of a Micromass Platform II spectrometer: ES Ionization in positive mode (mass range: 150-1200). The simultaneous chromatographic separation was achieved with the following HPLC system: ES Industries Chromegabond WR C-18 3u 120 Å (3.2×30 mm) column cartridge; Mobile Phase A: Water (0.02% TFA) and Phase B: Acetonitrile (0.02% TFA); gradient 10% B to 90% B in 3 minutes; equilibration time of 1 minute; flow rate of 2 mL/minute.

Many compounds of Formula 1 were also purified by reversed phased HPLC, using methods well known to those skilled in the art. In some cases, preparative HPLC purification was conducted using PE Sciex 150 EX Mass Spec controlling a Gilson 215 collector attached to a Shimadzu preparative HPLC system and a Leap autoinjector. Compounds were collected from the elution stream using LC/MS detection in the positive ion detection: The elution of compounds from C-18 columns (2.0×10 cm eluting at 20 mL/min) was effected using appropriate linear gradation mode over 10 minutes of Solvent (A) 0.05% TFA/H₂O and Solvent (B) 0.035% TFA/acetonitrile. For injection on to HPLC systems, the crude samples were dissolved in mixtures of methanol, acetonitrile and DMSO.

Compounds were characterized either by ¹H-NMR using a Bruker 400 MHz NMR Spectrometer.

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using Isis AutoNom 2000.

### Preparative Examples

### Example I-1

### 4-(4-Chloro-phenyl)-7H-pyrrolo[2,3-d]pyrimidine

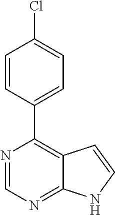 [SMILES: Clc1ccc(-c2ncnc3[nH]ccc23)cc1]

In a 10 mL microwave tube, 4-chloro-7H-pyrrolo[2,3-d]pyrimidine (200 mg, 1.3 mmol, Eq: 1.00), 4-chlorophenylboronic acid (204 mg, 1.3 mmol, Eq: 1.00) and potassium carbonate (720 mg, 5.21 mmol, Eq: 4.00) in 2 mL of water were combined with DME (4.00 ml). Pd (PPh₃)₄ was added (78 mg, 0.068 mmol). The reaction mixture was irradiated at 160° C. in a microwave for 60 minutes. The resulting solution was diluted with EtOAc, washed with water. The combined organic phases were dried over anhydrous sodium sulfate then evaporated. The crude material was dissolved with DCM and filtered. The title compound was obtained as a green solid (90 mg, 30% yield). LC/MS: m/z calculated for C₁₂H₈ClN₃([M+H]⁺): 230.6 Found: 230.1

### Example I-2

### 4-(3-Chloro-phenyl)-7H-pyrrolo[2,3-d]pyrimidine

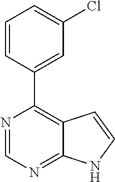 [SMILES: Clc1cccc(-c2ncnc3[nH]ccc23)c1]

Following a similar procedure as described in example 1 using 3-chlorophenylboronic acid, the title compound may be obtained.

### Example I-3

### 4-(2-Chloro-phenyl)-7H-pyrrolo[2,3-d]pyrimidine

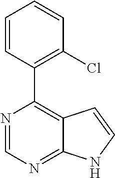 [SMILES: Clc1ccccc1-c1ncnc2[nH]ccc12]

Following a similar procedure as described in example 1 using 2-chlorophenylboronic acid, the title compound may be obtained.

### Example I-4

### 4-(3-Fluoro-4-methyl-phenyl)-7H-pyrrolo[2,3-d]pyrimidine

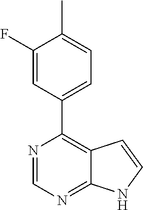 [SMILES: Cc1ccc(-c2ncnc3[nH]ccc23)cc1F]

Following a similar procedure as described in example 1 using 3-fluoro-4-methylphenylboronic acid, the title compound may be obtained.

### Example I-5

### 4-(2,4-Dimethyl-phenyl)-7H-pyrrolo[2,3-d]pyrimidine

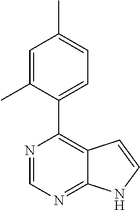 [SMILES: Cc1ccc(-c2ncnc3[nH]ccc23)c(C)c1]

In a 10 mL sealable microwave tube, 4-chloro-7-tosyl-7H-pyrrolo[2,3-d]pyrimidine (210 mg, 0.681 mmol, Eq: 1.00), 2,4-dimethylphenylboronic acid (112 mg, 0.749 mmol, Eq: 1.1) and potassium carbonate (376 mg, 2.72 mmol, Eq: 4.00) in 2 mL of water were combined with DME (4 mL). Pd(PPh₃)₄ (79 mg, 0.068 mmol, Eq: 0.1) was added. The reaction mixture was heated in a microwave at 160° C. for 60 minutes. The resulting solution was diluted with EtOAc and washed with brine. The combined organic phases were dried over anhydrous sodium sulfate then the solvent was removed under reduced pressure. The crude material was purified by column chromatography (silica, 5-70% ethyl acetate in hexanes).

The title compound was obtained as a solid (51 mg, 34% yield). LC/MS: m/z calculated for C₁₄H₁₃N₃([M+H]⁺): 224.2 Found: 224.2

### Example I-6

### 4-(3,4-Dimethyl-phenyl)-7H-pyrrolo[2,3-d]pyrimidine

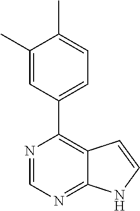 [SMILES: Cc1ccc(-c2ncnc3[nH]ccc23)cc1C]

In a 10 mL sealable microwave tube, 4-chloro-7-tosyl-7H-pyrrolo[2,3-d]pyrimidine (210 mg, 0.681 mmol, Eq: 1.00), 3,4-dimethylphenylboronic acid (112 mg, 0.749 mmol, Eq: 1.1) and potassium carbonate (376 mg, 2.72 mmol, Eq: 4.00) in 2 mL of water were combined with DME (4 mL). Pd(PPh₃)₄ (79 mg, 0.0681 mmol, Eq: 0.1) was added. The reaction mixture was heated in a microwave at 160° C. for 60 minutes. The resulting solution was diluted with EtOAc and washed with brine. The combined organic phases were dried over anhydrous sodium sulfate then the solvent was removed under reduced pressure. The crude material was purified by column chromatography (silica, 5-70% ethyl acetate in hexanes). The title compound was obtained as a solid (17 mg, 11% yield). LC/MS: m/z calculated for C₁₄H₁₃N₃([M+H]⁺): 224.2 Found: 224.2

### Example I-7

### 4-p-Tolyl-7H-pyrrolo[2,3-d]pyrimidine

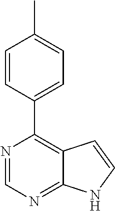 [SMILES: Cc1ccc(-c2ncnc3[nH]ccc23)cc1]

In a 10 mL sealable microwave tube, 4-chloro-7-tosyl-7H-pyrrolo[2,3-d]pyrimidine (210 mg, 0.681 mmol, Eq: 1.00), p-tolylboronic acid (102 mg, 0.749 mmol, Eq: 1.1) and potassium carbonate (376 mg, 2.72 mmol, Eq: 4.00) in 2 mL of water were combined with DME (4 mL). Pd(PPh₃)₄ (79 mg, 0.0681 mmol, Eq: 0.1) was added. The reaction mixture was heated in a microwave at 160° C. for 60 minutes. The resulting solution was diluted with EtOAc and washed with brine. The combined organic phases were dried over anhydrous sodium sulfate then the solvent was removed under reduced pressure. The crude material was purified by column chromatography (silica, 5-70% ethyl acetate in hexanes The title compound was obtained as a solid (60 mg, 42% yield). LC/MS: m/z calculated for C₁₃H₁₁N₃([M+H]⁺): 210.2 Found: 210.2

### Example I-8

### 4-(3-Chloro-4-methyl-phenyl)-7H-pyrrolo[2,3-d]pyrimidine

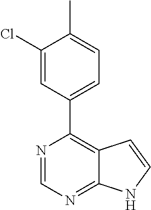 [SMILES: Cc1ccc(-c2ncnc3[nH]ccc23)cc1Cl]

In a 10 mL sealable microwave tube, 4-chloro-7-tosyl-7H-pyrrolo[2,3-d]pyrimidine (210 mg, 0.681 mmol, Eq: 1.00), 3-chloro-4-methylphenylboronic acid (128 mg, 0.749 mmol, Eq: 1.1) and potassium carbonate (376 mg, 2.72 mmol, Eq: 4.00) in 2 mL of water were combined with DME (4 mL). Pd(PPh₃)₄ (79 mg, 0.068 mmol, Eq: 0.1) was added. The reaction mixture was heated in a microwave at 160° C. for 60 minutes. The resulting solution was diluted with EtOAc and washed with brine. The combined organic phases were dried over anhydrous sodium sulfate then the solvent was removed under reduced pressure. The crude material was triturated with DCM. The solid was filtered. The title compound was obtained as a solid (14 mg, 8% yield). LC/MS: m/z calculated for C₁₃H₁₀ClN₃([M+H]⁺): 244.7 Found: 244.2

### Example I-9

### 4-tert-Butyl-N-[4-(7H-pyrrolo[2,3-d]pyrimidin-4-yl)-benzyl]-benzamide

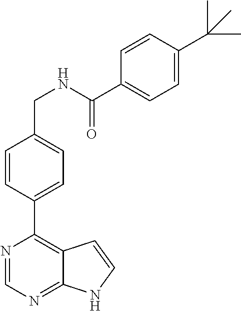 [SMILES: CC(C)(C)c1ccc(C(=O)NCc2ccc(-c3ncnc4[nH]ccc34)cc2)cc1]

### Step 1: [4-(7H-Pyrrolo[2,3-d]pyrimidin-4-yl)-benzyl]-carbamic acid tert-butyl ester

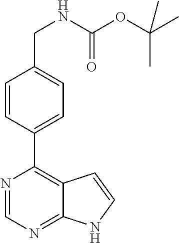 [SMILES: CC(C)(C)OC(=O)NCc1ccc(-c2ncnc3[nH]ccc23)cc1]

In a 20 mL sealable microwave tube, 4-chloro-7H-pyrrolo[2,3-d]pyrimidine (500 mg, 3.26 mmol, Eq: 1.00), tert-butyl 4-(4,4,5,5-tetramethyl-1,3,2-dioxaborolan-2-yl)benzylcarbamate (1.6 g, 4.8 mmol, Eq: 1.47) and potassium carbonate (1.8 g, 13.0 mmol, Eq: 4.00) were combined with DME (10 mL) and water (5 mL). Pd(PPh₃)₄ (376 mg, 0.326 mmol, Eq: 0.1) was added and the reaction mixture was heated at 150° C. for 60 minutes. The resulting solution was diluted with EtOAc and washed with brine. The combined organic phases were dried over anhydrous sodium sulfate then the solvent was removed under reduced pressure. The crude material was purified by column chromatography (silica, 5-70% ethyl acetate in hexanes). The title compound was obtained as a solid (545 mg, 52% yield). LC/MS: m/z calculated for C₁₈H₂₀N₄O₂([M+H]⁺): 325.3 Found: 325.2

### Step 2: 4-tert-Butyl-N-[4-(7H-pyrrolo[2,3-d]pyrimidin-4-yl)-benzyl]-benzamide

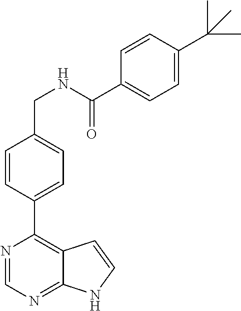 [SMILES: CC(C)(C)c1ccc(C(=O)NCc2ccc(-c3ncnc4[nH]ccc34)cc2)cc1]

In a 20 mL scintillation vial, tert-butyl 4-(7H-pyrrolo[2,3-d]pyrimidin-4-yl)benzylcarbamate (200 mg, 0.617 mmol, Eq: 1.00) was dissolved with 2 mL DCM and 2 mL of TFA. The reaction mixture was stirred at room temperature for 30 minutes. The solvent was removed under reduced pressure. The crude material was dissolved with DMF (4 mL). 4-tert-butylbenzoic acid (121 mg, 0.678 mmol, Eq: 1.1), DIPEA (0.43 mL, 2.47 mmol, Eq: 4.00) and HATU (258 mg, 0.678 mmol, Eq: 1.1) were added. The reaction mixture was stirred at room temperature overnight. The reaction mixture was diluted with EtOAc and water, then stirred at room temperature for 30 minutes. The resulting solution was washed with brine. The combined organic phases were dried over anhydrous sodium sulfate then the solvent was removed under reduced pressure. The crude material was triturated with DCM and the solid obtained was filtered to provide the title compound as a solid (65 mg, 27% yield). LC/MS: m/z calculated for C₂₄H₂₄N₄O([M+H]⁺): 385.4 Found: 385.1

### Example I-10

### 3-Chloro-N-[4-(7H-pyrrolo[2,3-d]pyrimidin-4-yl)-benzyl]-benzamide

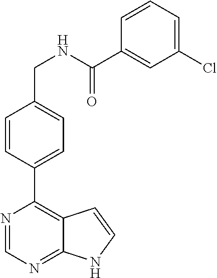 [SMILES: O=C(NCc1ccc(-c2ncnc3[nH]ccc23)cc1)c1cccc(Cl)c1]

Following a similar procedure described in example 9, step 2, using 3-chlorobenzoic acid (96.5 mg, 0.617 mmol, Eq: 1.00), the title compound was obtained as solid (45 mg, 20% yield). LC/MS: m/z calculated for C₂₀H₁₅ClN₄O([M+H]⁺): 363.8 Found: 363.0

### Example I-11

### 2-(3-Chloro-phenylamino)-N-[4-(7H-pyrrolo[2,3-d]pyrimidin-4-yl)-benzyl]-acetamide

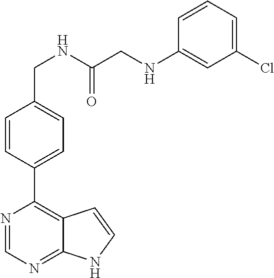 [SMILES: O=C(CNc1cccc(Cl)c1)NCc1ccc(-c2ncnc3[nH]ccc23)cc1]

### Step 1: (3-Chloro-phenylamino)-acetic acid methyl ester

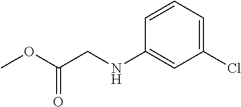

In a 250 mL round-bottomed flask, methyl bromoacetate (1.66 g, 1 mL, 10.9 mmol, Eq: 1.00), 3-chloroaniline (1.66 g, 1.4 mL, 13.0 mmol, Eq: 1.2) and DIPEA (1.9 mL, 10.9 mmol, Eq: 1.00) were combined with DMF (20 mL) to give a light yellow solution. The reaction mixture was heated at 60° C. overnight. The reaction mixture was diluted with EtOAc then washed with brine. The combined organic phases were dried over anhydrous sodium sulfate. The solvent was removed under reduced pressure to afford title compound (2.1 g, 97% yield). LC/MS: m/z calculated for C₉H₁₀ClNO₂ ([M+H]⁺): 200.6 Found: 200.0

### Step 2: (3-Chloro-phenylamino)-acetic acid

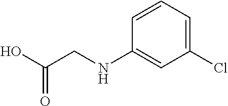 [SMILES: O=C(O)CNc1cccc(Cl)c1]

In a 20 mL scintillation vial, (3-chloro-phenylamino)-acetic acid methyl ester (500 mg, 2.5 mmol, Eq: 1.00) and NaOH (500 mg, 12.5 mmol, Eq: 4.99) in 5 mL H₂O were combined with EtOH (8 mL) to give a light yellow solution. The reaction mixture was heated at 60° C. for 4 hours. The reaction mixture was diluted with EtOAc and washed with 10% aqueous HCl. The combined organic phases were dried over anhydrous sodium sulfate then evaporated under reduced pressure to afford title compound as a brown solid. LC/MS: m/z calculated for C₈H₈ClNO₂ ([M+H]⁺): 186.0 Found: 186.0

### Step 3: 2-(3-Chloro-phenylamino)-N-[4-(7H-pyrrolo[2,3-d]pyrimidin-4-yl)-benzyl]-acetamide

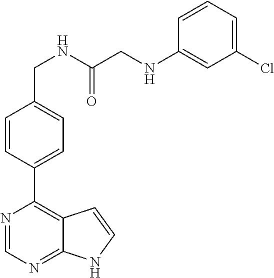 [SMILES: O=C(CNc1cccc(Cl)c1)NCc1ccc(-c2ncnc3[nH]ccc23)cc1]

Following a similar procedure described in example 9, step 2, using tert-butyl 4-(7H-pyrrolo[2,3-d]pyrimidin-4-yl)benzylcarbamate (345 mg, 1.06 mmol, Eq: 1.00), 2-(3-chlorophenylamino)acetic acid (217 mg, 1.17 mmol, Eq: 1.1), DIPEA (740 mg, 1 mL, 5.73 mmol, Eq: 5.38) and HATU (445 mg, 1.17 mmol, Eq: 1.1), the title compound was obtained as a solid (71 mg, 17% yield). LC/MS: m/z calculated for C₂₁H₁₈ClN₅O([M+H]⁺): 392.8 Found: 392.1

### Example I-12

### 4-tert-Butyl-N-[2-fluoro-4-(7H-pyrrolo[2,3-d]pyrimidin-4-yl)-benzyl]-benzamide

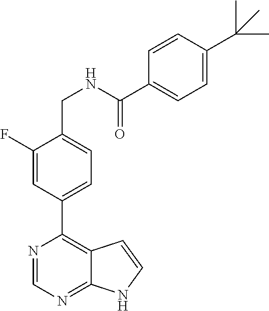 [SMILES: CC(C)(C)c1ccc(C(=O)NCc2ccc(-c3ncnc4[nH]ccc34)cc2F)cc1]

### Step 1: [2-Fluoro-4-(4,4,5,5-tetramethyl-[1,3,2]dioxaborolan-2-yl)-benzyl]-carbamic acid tert-butyl ester

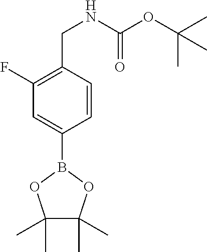 [SMILES: CC(C)(C)OC(=O)NCc1ccc(B2OC(C)(C)C(C)(C)O2)cc1F]

In a pressure tube, tert-butyl 4-bromo-2-fluorobenzylcarbamate (5 g, 16.4 mmol), bis(pinacolato)diboron (6.26 g, 24.7 mmol) and potassium acetate (4.84 g, 49.3 mmol) were combined with NMP (75.0 mL) to give a light yellow solution. The reaction mixture was degassed under nitrogen for 10 minutes. [1,1′-Bis (diphenylphosphino)ferrocene]dichloro-palladium(II) (722 mg, 0.986 mmol) was added. The reaction mixture was heated at 100° C. for 20 hours. The reaction mixture was quenched with water, and extracted with DCM (3×100 mL). The combined organic layers were washed with water, brine, dried over Na₂SO₄, filtered, and concentrated. The crude material was purified by flash chromatography (silica gel, 120 g, 0% to 30% ethyl acetate in hexanes). [2-Fluoro-4-(4,4,5,5-tetramethyl-[1,3,2]dioxaborolan-2-yl)-benzyl]-carbamic acid tert-butyl ester (5.8 g, 100%) was obtained as a yellow oil.

### Step 2: [2-Fluoro-4-(7H-pyrrolo[2,3-d]pyrimidin-4-yl)-benzyl]-carbamic acid tert-butyl ester

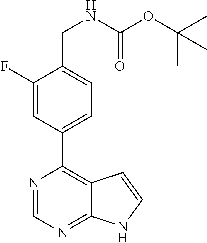 [SMILES: CC(C)(C)OC(=O)NCc1ccc(-c2ncnc3[nH]ccc23)cc1F]

In a 10 mL sealable microwave tube, 4-chloro-7-tosyl-7H-pyrrolo[2,3-d]pyrimidine (157 mg, 0.511 mmol, Eq: 1.00), [2-Fluoro-4-(4,4,5,5-tetramethyl-[1,3,2]dioxaborolan-2-yl)-benzyl]-carbamic acid tert-butyl ester (197 mg, 0.562 mmol, Eq: 1.1) and potassium carbonate (282 mg, 2.04 mmol, Eq: 4.00) in 2 mL of water were combined with DME (4 mL). Pd(Ph₃P)₄ (59.0 mg, 0.051 mmol, Eq: 0.1) was added and the reaction mixture was heated at 160° C. for 60 minutes. The resulting solution was diluted with EtOAc and washed with brine. The combined organic phases were dried over anhydrous sodium sulfate then the solvent was removed under reduced pressure. The crude material was purified by column chromatography (silica, 5-70% ethyl acetate in hexanes). The title compound was obtained as a solid (75 mg, 43% yield). LC/MS: m/z calculated for C₁₈H₁₉FN₄O₂([M+H]⁺): 343.3 Found: 343.3

### Step 3: 4-tert-Butyl-N-[2-fluoro-4-(7H-pyrrolo[2,3-d]pyrimidin-4-yl)-benzyl]-benzamide

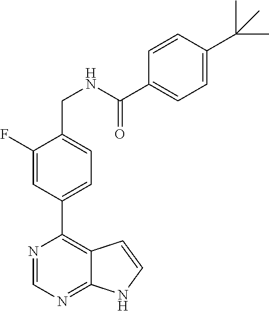 [SMILES: CC(C)(C)c1ccc(C(=O)NCc2ccc(-c3ncnc4[nH]ccc34)cc2F)cc1]

In a 20 mL scintillation vial, [2-Fluoro-4-(7H-pyrrolo[2,3-d]pyrimidin-4-yl)-benzyl]carbamic acid tert-butyl ester (70 mg, 0.134 mmol, Eq: 1.00) was dissolved with 2 mL DCM and 2 mL of TFA. The reaction mixture was stirred at room temperature for 30 minutes. The solvent was removed under reduced pressure. The crude material was dissolved with DMF (4 mL). 4-tert-butylbenzoic acid (26 mg, 0.148 mmol, Eq: 1.1), DIPEA (0.047 mL, 0.269 mmol, Eq: 4.00) and HATU (56 mg, 0.148 mmol, Eq: 1.1) were added. The reaction mixture was stirred at room temperature overnight. The reaction mixture was diluted with EtOAc and water, then stirred at room temperature for 30 minutes. The resulting solution was washed with brine. The combined organic phases were dried over anhydrous sodium sulfate then the solvent was removed under reduced pressure. The crude material was triturated with DCM and the solid obtained was filtered to provide the title compound as a solid (32 mg, 59% yield). LC/MS: m/z calculated for C₂₄H₂₃FN₄O([M+H]⁺): 403.4 Found: 403.2

### Example I-13

### 4-{4-[(4-tert-Butyl-benzoylamino)-methyl]-3-fluoro-phenyl}-7H-pyrrolo[2,3-d]pyrimidine-6-carboxylic acid tert-butyl ester

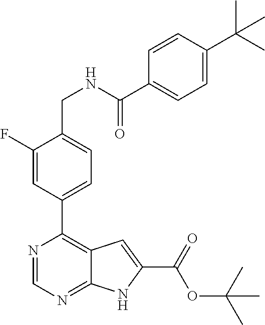 [SMILES: CC(C)(C)OC(=O)c1cc2c(-c3ccc(CNC(=O)c4ccc(C(C)(C)C)cc4)c(F)c3)ncnc2[nH]1]

### Step 1: 4-Chloro-5-hydroxy-6,7-dihydro-5H-pyrrolo[2,3-d]pyrimidine-6-carboxylic acid tert-butyl ester

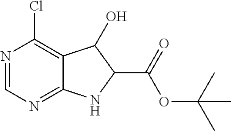 [SMILES: CC(C)(C)OC(=O)C1Nc2ncnc(Cl)c2C1O]

To a suspension of 4,6-dichloropyrimidine-5-carbaldehyde (2 g, 11.3 mmol, Eq: 1.00) in EtOH (50 mL) was added tert-butyl 2-aminoacetate (1.48 g, 11.3 mmol, Eq: 1.00) followed by triethylamine (2.86 g, 3.94 mL, 28.3 mmol, Eq: 2.5) and stirred at r.t. for 48 h. The solvent was removed under reduced pressure. The crude material was diluted with dichloromethane and washed with water. The combined organic phased were dried over anhydrous sodium sulfate and the solvent was removed under reduced pressure. The crude material was purified by column chromatography (silica, 10-90% ethyl acetate in hexanes) to give title compound (634 mg, 21% yield) as a white solid. LC/MS: m/z calculated for C₁₁H₁₄ClN₃O([M+H]⁺): 272.7 Found: 272.1

### Step 2: 4-Chloro-7H-pyrrolo[2,3-d]pyrimidine-6-carboxylic acid tert-butyl ester

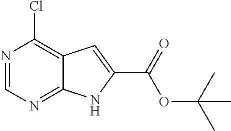 [SMILES: CC(C)(C)OC(=O)c1cc2c(Cl)ncnc2[nH]1]

To a solution of tert-butyl 4-chloro-5-hydroxy-6,7-dihydro-5H-pyrrolo[2,3-d]pyrimidine-6-carboxylate (634 mg, 2.33 mmol, Eq: 1.00) in DMF (10 mL) was added sodium hydride (93.3 mg, 2.33 mmol, Eq: 1.00) at 0° C. and then stirred at r.t. for 1 h. The reaction was quenched with water then washed with NH₄Cl and brine. The combined organic layers were dried over anhydrous sodium sulfate then the solvent was removed under reduced pressure. The crude material was purified by column chromatography (silica, 5-35% ethyl acetate in hexanes) to give title compound (417 mg, 70.4% yield) as a white solid. LC/MS: m/z calculated for C₁₁H₁₂ClN₃O([M+H]⁺): 254.6 Found: 254.1

### Step 3: N-(4-Bromo-2-fluoro-benzyl)-4-tert-butyl-benzamide

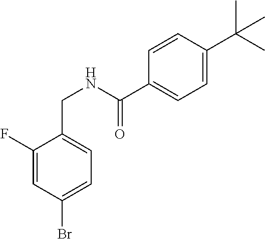 [SMILES: CC(C)(C)c1ccc(C(=O)NCc2ccc(Br)cc2F)cc1]

To a solution of (4-bromo-2-fluorophenyl)methanamine (1.5 g, 7.35 mmol, Eq: 1.00) in DCM (25 mL) cooled to 0° C. was added a solution of 4-tert-butylbenzoyl chloride (1.45 g, 7.35 mmol, Eq: 1.00), triethylamine (1.49 g, 2.05 mL, 14.7 mmol, Eq: 2.00) in DCM (5 mL). The reaction mixture was warmed to r.t. for 1 hr. The reaction mixture was purified by column chromatography (silica, 5-40% ethyl acetate in hexanes) to give title compound (2.59 g, 7.11 mmol, 96.7% yield) as a white solid. ¹H NMR (400 MHz, CHLOROFORM-d) δ ppm 7.74 (d, J=8.0 Hz, 2H), 7.48 (d, J=8.5 Hz, 2H), 7.35 (t, J=7.5 Hz, 1H), 6.48 (s, 1H), 4.67 (d, J=5.7 Hz, 2H), 1.36 (s, 9H).

### Step 4: 4-tert-Butyl-N-[2-fluoro-4-(4,4,5,5-tetramethyl-[1,3,2]dioxaborolan-2-yl)-benzyl]-benzamide

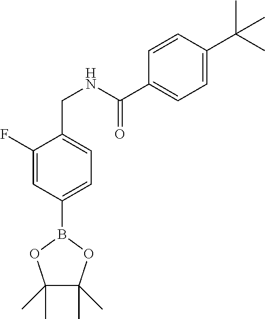 [SMILES: CC(C)(C)c1ccc(C(=O)NCc2ccc(B3OC(C)(C)C(C)(C)O3)cc2F)cc1]

To a mixture of N-(4-bromo-2-fluorobenzyl)-4-tert-butylbenzamide (600 mg, 1.65 mmol, Eq: 1.00), 4,4,4′,4′,5,5,5′,5′-octamethyl-2,2′-bi(1,3,2-dioxaborolane) (627 mg, 2.47 mmol, Eq: 1.5), potassium acetate (485 mg, 4.94 mmol, Eq: 3) and PdCl₂(dppf)-CH₂Cl₂ (121 mg, 165 μmol, Eq: 0.1) stirring under N₂ was added NMP (12 mL) and heated to 100° C. for 16 h. The reaction mixture was diluted with EtOAc and washed with water and brine. The combined organic layers were dried over anhydrous sodium sulfate and the solvent was removed under reduced pressure. The crude material was purified by column chromatography (silica, 15-60% ethyl acetate in hexanes) to give title compound (545 mg, 80% yield) as an off-white solid. ¹H NMR (400 MHz, CHLOROFORM-d) δ ppm 7.74 (d, J=8.3 Hz, 2H), 7.75 (d, J=7.4 Hz, 1H), 7.51 (d, J=10.4 Hz, 1H), 7.47 (d, J=8.4 Hz, 2H), 7.44 (t, J=7.0 Hz, 1H), 6.46 (s, 1H), 4.74 (d, J=5.9 Hz, 2H), 1.37 (s, 12H), 1.36 (s, 9H).

### Step 5: 4-{4-[(4-tert-Butyl-benzo ylamino)-methyl]-3-fluoro-phenyl}-7H-pyrrolo[2,3-d]pyrimidine-6-carboxylic acid tert-butyl ester

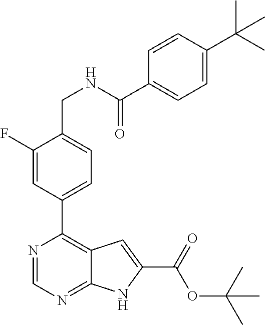 [SMILES: CC(C)(C)OC(=O)c1cc2c(-c3ccc(CNC(=O)c4ccc(C(C)(C)C)cc4)c(F)c3)ncnc2[nH]1]

To a mixture of tert-butyl 4-chloro-7H-pyrrolo[2,3-d]pyrimidine-6-carboxylate (100 mg, 0.394 mmol, Eq: 1.00), 4-tert-butyl-N-(2-fluoro-4-(4,4,5,5-tetramethyl-1,3,2-dioxaborolan-2-yl)benzyl)benzamide (162 mg, 0.394 mmol, Eq: 1.00), tetrakis(triphenylphosphine)palladium (0) (46 mg, 0.04 mmol, Eq: 0.1) and potassium carbonate (163 mg, 1.18 mmol, Eq: 3.00) was added DME (1 mL) and water (500 μL) and heated in the microwave at 150° C. for 30 min. The reaction mixture was filtered through a pad of celite and diluted with dichloromethane. The solution was purified by column chromatography (silica, 15-60% ethyl acetate in hexanes followed by 0-30% [10% Methanol/dichloromethane] in dichloromethane) to give title compound (80 mg, 40% yield) as an off-white solid. ¹H NMR (400 MHz, CHLOROFORM-d) δ ppm 10.93 (s, 1H), 9.18 (s, 1H), 7.94 (t, J=9.3 Hz, 2H), 7.80 (d, J=8.5 Hz, 2H), 7.68 (t, J=7.8 Hz, 1H), 7.50 (d, J=8.4 Hz, 1H), 7.39 (s, 1H), 6.67 (t, J=5.9 Hz, 1H), 4.83 (d, J=5.8 Hz, 2H), 1.69 (s, 9H), 1.37 (s, 9H); LC/MS: m/z calculated for C₂₉H₃₁FN₄O₃([M+H]⁺): 503.5 Found: 503.3

### Example I-14

### 4-{4-[(4-tert-Butyl-benzoylamino)-methyl]-3-fluoro-phenyl}-7H-pyrrolo[2,3-d]pyrimidine-6-carboxylic acid

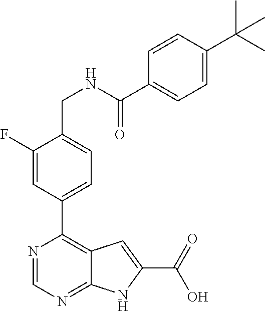 [SMILES: CC(C)(C)c1ccc(C(=O)NCc2ccc(-c3ncnc4[nH]c(C(=O)O)cc34)cc2F)cc1]

To a solution of 4-{4-[(4-tert-Butyl-benzoylamino)-methyl]-3-fluoro-phenyl}-7H-pyrrolo[2,3-d]pyrimidine-6-carboxylic acid tert-butyl ester (55 mg, 0.109 mmol, Eq: 1.00) in dichloromethane (1 mL) was added trifluoroacetic acid (843 μL, 10.9 mmol, Eq: 100) and stirred at r.t. for 2 h. The solvent was concentrated in vacuo from methanol (3×) to give title compound (43 mg, 88% yield) as a light brown solid. ¹H NMR (400 MHz, DMSO-d₆) δ ppm 13.05 (s, 1H), 9.10 (t, J=5.9 Hz, 1H), 8.98 (s, 1H), 8.06 (d, J=8.2 Hz, 1H), 7.97 (d, J=11.2 Hz, 1H), 7.89 (d, J=8.4 Hz, 2H), 7.59 (t, J=7.6 Hz, 1H), 7.54 (m, 3H), 4.64 (d, J=5.6 Hz, 2H), 1.33 (s, 9H); LC/MS: m/z calculated for C₂₅H₂₃FN₄O₃([M+H]⁺): 447.4 Found: 447.2

### Example I-15

### 4-tert-butyl-N-(2-fluoro-4-(6-(morpholine-4-carbonyl)-7H-pyrrolo[2,3-d]pyrimidin-4-yl)benzyl)benzamide

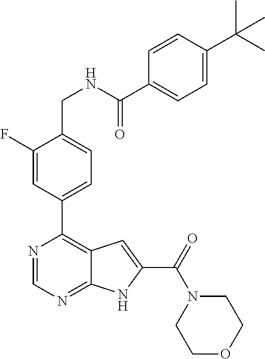 [SMILES: CC(C)(C)c1ccc(C(=O)NCc2ccc(-c3ncnc4[nH]c(C(=O)N5CCOCC5)cc34)cc2F)cc1]

To a solution of 4-{4-[(4-tert-Butyl-benzoylamino)-methyl]-3-fluoro-phenyl}-7H-pyrrolo[2,3-d]pyrimidine-6-carboxylic acid (50 mg, 0.112 mmol, Eq: 1.00), HBTU (42.5 mg, 0.112 mmol, Eq: 1.00) and DIPEA (59 μl, 0.336 mmol, Eq: 3) in DMF (1.5 mL) was added morpholine (19.5 mg, 0.224 mmol, Eq: 2.00) and stirred at room temperature for 16 h. The reaction mixture was diluted with ethyl acetate and washed with brine. The combined organic phases were dried over anhydrous sodium sulfate then the solvent was removed under reduced pressure. The crude material was purified by column chromatography (silica, 0-5% methanol in dichloromethane followed by 50-100% ethyl acetate in hexanes) to give title compound (26 mg, 45% yield) as a white solid. ¹H NMR (400 MHz, CHLOROFORM-d) δ ppm 8.96 (s, 1H), 7.74 (m, 2H), 7.71 (d, J=8.4 Hz, 2H), 7.58 (t, J=8.2 Hz, 1H), 7.41 (d, J=8.4 Hz, 1H), 6.91 (s, 1H), 4.71 (s, 1H), 3.81 (br. s, 4H), 3.72 (br. s, 4H), 1.27 (s, 9H).

### Example I-16

### 4-{4-[(4-tert-Butyl-benzoylamino)-methyl]-3-fluoro-phenyl}-7H-pyrrolo[2,3-d]pyrimidine-6-carboxylic acid dimethylamide

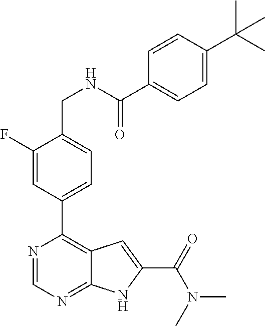 [SMILES: CN(C)C(=O)c1cc2c(-c3ccc(CNC(=O)c4ccc(C(C)(C)C)cc4)c(F)c3)ncnc2[nH]1]

To a solution of 4-{4-[(4-tert-Butyl-benzoylamino)-methyl]-3-fluoro-phenyl}-7H-pyrrolo[2,3-d]pyrimidine-6-carboxylic acid (50 mg, 0.112 mmol, Eq: 1.00), HBTU (42.5 mg, 0.112 mmol, Eq: 1.00) and DIPEA (59 μl, 0.336 mmol, Eq: 3.00) in DMF (1 mL) was added dimethylamine in THF (112 μl, 0.224 mmol, Eq: 2.00) and stirred at room temperature for 16 h. The reaction mixture was diluted with ethyl acetate and washed with brine. The combined organic phases were dried over anhydrous sodium sulfate then the solvent was removed under reduced pressure. The crude material was purified by column chromatography (silica, 50-100% ethyl acetate in hexanes gradient) to give title compound (18 mg, 34% yield) as a white solid. ¹H NMR (400 MHz, DMSO-d) 6 ppm 12.78 (s, 1H), 9.10 (t, J=5.8 Hz, 1H), 8.92 (s, 1H), 8.04 (d, J=8.0 Hz, 1H), 7.96 (d, J=11.6 Hz, 1H), 7.89 (d, J=8.6 Hz, 2H), 7.56 (t, J=7.9 Hz, 1H), 7.53 (d, J=8.3 Hz, 2H), 7.23 (s, 1H), 4.62 (s, 2H), 3.26 (br. s, 3H), 3.06 (br. s, 3H), 1.32 (s, 9H); LC/MS: m/z calculated for C₂₇H₂₈FN₅O₂([M+H]⁺): 474.5 Found: 474.3

### Example I-17

### 4-{4-[(4-tert-Butyl-benzoylamino)-methyl]-3-fluoro-phenyl}-7H-pyrrolo[2,3-d]pyrimidine-6-carboxylic acid methylamide

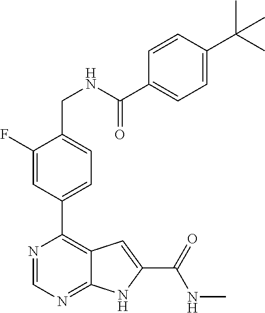 [SMILES: CNC(=O)c1cc2c(-c3ccc(CNC(=O)c4ccc(C(C)(C)C)cc4)c(F)c3)ncnc2[nH]1]

To a solution of 4-{4-[(4-tert-Butyl-benzoylamino)-methyl]-3-fluoro-phenyl}-7H-pyrrolo[2,3-d]pyrimidine-6-carboxylic acid (50 mg, 0.112 mmol, Eq: 1.00), HBTU (42.5 mg, 0.112 mmol, Eq: 1.00) and DIPEA (43 mg, 59 μl, 0.336 mmol, Eq: 3.00) in DMF (1.00 mL) was added methanamine in THF (112 μl, 0.224 mmol, Eq: 2.00) and stirred at room temperature for 16 h. The reaction mixture was diluted with ethyl acetate and washed with brine. The combined organic phases were dried over anhydrous sodium sulfate then the solvent was removed under reduced pressure. The crude material was purified by column chromatography (silica, 50-100% ethyl acetate in hexanes) to give title compound (25 mg, 48.6% yield) as a white solid. ¹H NMR (400 MHz, DMSO-d₆) δ ppm 12.82 (s, 1H), 9.11 (t, J=5.7 Hz, 1H), 8.92 (s, 1H), 8.70 (d, J=4.8 Hz, 1H), 8.01 (d, J=7.8 Hz, 1H), 7.93 (d, J=11.6 Hz, 1H), 7.89 (d, J=8.7 Hz, 2H), 7.61 (s, 1H), 7.59 (t, J=8.1 Hz, 1H), 7.53 (d, J=8.4 Hz, 2H), 4.64 (d, J=5.4 Hz, 2H), 2.84 (d, J=4.5 Hz, 3H), 1.32 (s, 9H); LC/MS: m/z calculated for C₂₆H₂₆FN₅O₂([M+H]⁺): 460.5 Found: 460.3

### Example I-18

### 4-{4-[(4-tert-Butyl-benzoylamino)-methyl]-3-fluoro-phenyl}-7H-pyrrolo[2,3-d]pyrimidine-6-carboxylic acid (2-hydroxy-ethyl)-amide

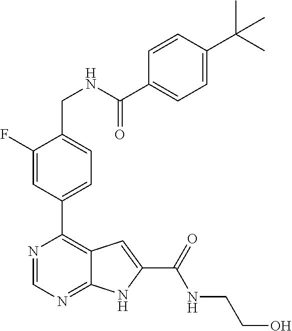 [SMILES: CC(C)(C)c1ccc(C(=O)NCc2ccc(-c3ncnc4[nH]c(C(=O)NCCO)cc34)cc2F)cc1]

To a solution of 4-{4-[(4-tert-Butyl-benzoylamino)-methyl]-3-fluoro-phenyl}-7H-pyrrolo[2,3-d]pyrimidine-6-carboxylic acid (50 mg, 0.112 mmol, Eq: 1.00), HBTU (42.5 mg, 0.112 mmol, Eq: 1.00) and DIPEA (59 μl, 0.336 mmol, Eq: 3.00) in DMF (1.00 mL) was added 2-aminoethanol (14 mg, 0.224 mmol, Eq: 2.00) and stirred at room temperature for 16 h. The reaction mixture was diluted with ethyl acetate and washed with brine. The combined organic phases were dried over anhydrous sodium sulfate then the solvent was removed under reduced pressure. The crude material was purified by column chromatography (silica, 50-100% ethyl acetate in hexanes followed by 0-10% methanol in dichloromethane containing NH₄OH) to give title compound (16 mg, 29% yield) as an off-white solid. ¹H NMR (400 MHz, DMSO-d₆) δ ppm 12.80 (s, 1H), 9.10 (t, J=5.6 Hz, 1H), 8.92 (s, 1H), 8.73 (t, J=6.4 Hz, 1H), 8.03 (d, J=8.6 Hz, 1H), 7.95 (d, J=11.0 Hz, 1H), 7.89 (d, J=8.4 Hz, 2H), 7.69 (s, 1H), 7.59 (t, J=7.5 Hz, 1H), 7.53 (d, J=8.2 Hz, 2H), 4.64 (d, J=6.0 Hz, 2H), 3.55 (t, J=5.2 Hz, 2H), 3.38 (m, 2H), 1.32 (s, 9H); LC/MS: m/z calculated for C₂₇H₂₈FN₅O₃([M+H]⁺): 490.5 Found: 490.4

### Example I-19

### 4-{4-[(4-tert-Butyl-benzoylamino)-methyl]-3-fluoro-phenyl}-7H-pyrrolo[2,3-d]pyrimidine-6-carboxylic acid (2-dimethylamino-ethyl)-amide

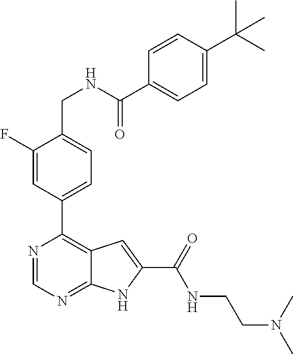 [SMILES: CN(C)CCNC(=O)c1cc2c(-c3ccc(CNC(=O)c4ccc(C(C)(C)C)cc4)c(F)c3)ncnc2[nH]1]

To a solution of 4-{4-[(4-tert-Butyl-benzoylamino)-methyl]-3-fluoro-phenyl}-7H-pyrrolo[2,3-d]pyrimidine-6-carboxylic acid (50 mg, 0.112 mmol, Eq: 1.00), N¹,N¹-dimethylethane-1,2-diamine (10 mg, 0.112 mmol, Eq: 1.00) and DIPEA (49 μl, 0.280 mmol, Eq: 2.5) in DMF (1.00 mL) cooled to 0° C. was added 1-propanephosphonic acid cyclic anhydride (80 μl, 0.134 mmol, Eq: 1.2) and allowed to warm to r.t. for 4 h. The reaction mixture was diluted with ethyl acetate and washed with brine. The combined organic phases were dried over anhydrous sodium sulfate then the solvent was removed under reduced pressure. The crude material was purified by column chromatography (0-15% methanol in dichloromethane cont. NH₄OH) to give title compound (25 mg, 43% yield) as an off-white solid. ¹H NMR (400 MHz, METHANOL-d₄) 6 ppm 8.93 (s, 1H), 7.97 (d, J=8.1 Hz, 1H), 7.91 (d, J=11.0 Hz, 1H), 7.86 (d, J=8.5 Hz, 2H), 7.66 (t, J=7.8 Hz, 1H), 7.57 (s, 1H), 7.56 (d, J=8.5 Hz, 2H), 4.76 (s, 2H), 3.75 (t, J=6.1 Hz, 2H), 3.22 (m, 2H), 2.84 (s, 6H), 1.38 (s, 9H); LC/MS: m/z calculated for C₂₉H₃₃FN₆O₂([M+H]⁺): 517.6 Found: 517.4

### Example I-20

### 4-tert-Butyl-N-{1-[6-(1-methyl-1H-pyrazol-4-yl)-7H-pyrrolo[2,3-d]pyrimidin-4-yl]-piperidin-4-ylmethyl}-benzamide

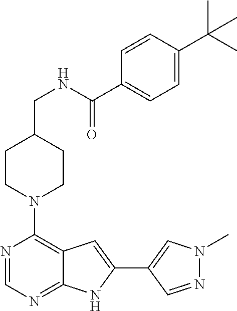 [SMILES: Cn1cc(-c2cc3c(N4CCC(CNC(=O)c5ccc(C(C)(C)C)cc5)CC4)ncnc3[nH]2)cn1]

### Step 1: 4-[(4-tert-Butyl-benzoylamino)-methyl]-piperidine-1-carboxylic acid tert-butyl ester

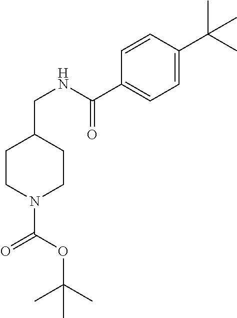 [SMILES: CC(C)(C)OC(=O)N1CCC(CNC(=O)c2ccc(C(C)(C)C)cc2)CC1]

To a solution of tert-butyl 4-(aminomethyl)piperidine-1-carboxylate (1 g, 4.67 mmol, Eq: 1.00) in DCM (16.7 ml) cooled to 0° C. was added a solution of 4-tert-butylbenzoyl chloride (918 mg, 4.67 mmol, Eq: 1.00), triethylamine (361 μl, 4.67 mmol, Eq: 1.00) in DCM (5 mL). The reaction mixture was warmed to r.t. for 1 h. The reaction mixture was purified by column chromatography (30-70% ethyl acetate in hexanes) to give title compound (1.75 g, 4.67 mmol, 100% yield) as a colorless viscous oil. ¹H NMR (400 MHz, CHLOROFORM-d) δ ppm 7.73 (d, J=8.5 Hz, 2H), 7.48 (d, J=8.1 Hz, 2H), 6.21 (s, 1H), 4.15 (d, J=12.9 Hz, 2H), 3.39 (t, J=6.0 Hz, 2H), 2.72 (t, J=14.7 Hz, 2H), 1.83 (m, 1H), 1.76 (d, J=14.3 Hz, 2H), 1.48 (s, 9H), 1.36 (s, 9H).

### Step 2: 4-tert-Butyl-N-piperidin-4-ylmethyl-benzamide

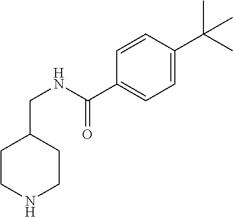 [SMILES: CC(C)(C)c1ccc(C(=O)NCC2CCNCC2)cc1]

To a solution of 4-[(4-tert-butyl-benzoylamino)-methyl]piperidine-1-carboxylic acid tert-butyl ester (1.75 g, 4.67 mmol, Eq: 1.00) in DCM (35 mL) was added trifluoroacetic acid (7.2 mL, 93.5 mmol, Eq: 20) and stirred at r.t. for 4 h. The solvent was removed by reduced pressure and dried in vacuo to give title compound (2.92 g, 124% yield) as a viscous colorless oil. ¹H NMR (400 MHz, CHLOROFORM-d) δ ppm 8.72 (br. s, 3H), 7.71 (d, J=8.5 Hz, 2H), 7.49 (d, J=8.5 Hz, 2H), 6.81 (t, J=6.7 Hz, 1H), 3.56 (d, J=12.8 Hz, 2H), 3.46 (t, J=6.4 Hz, 2H), 3.01 (q, J=11.7 Hz, 2H), 2.02 (d, J=14.3 Hz, 2H), 1.69 (q, J=14.1 Hz, 2H), 1.36 (s, 9H).

### Step 3: N-[1-(7-Benzenesulfonyl-6-iodo-7H-pyrrolo[2,3-d]pyrimidin-4-yl)-piperidin-4-ylmethyl]-4-tert-butyl-benzamide

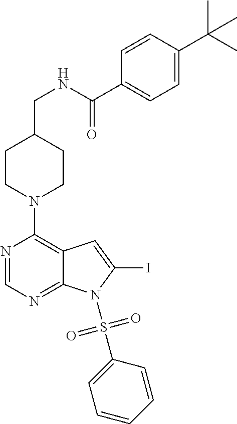 [SMILES: CC(C)(C)c1ccc(C(=O)NCC2CCN(c3ncnc4c3cc(I)n4S(=O)(=O)c3ccccc3)CC2)cc1]

To a suspension of 4-chloro-6-iodo-7-(phenylsulfonyl)-7H-pyrrolo[2,3-d]pyrimidine (200 mg, 0.477 mmol, Eq: 1.00) in EtOH (3.00 mL) was added 4-tert-butyl-N-piperidin-4-ylmethyl-benzamide (185 mg, 477 μmol, Eq: 1.00) and triethylamine (332 μl, 2.38 mmol, Eq: 5.00) and heated to 80° C. for 2 h. The reaction mixture was cooled to r.t. A precipitate was formed and was filtered off. The filtrate was purified by column chromatography (silica, 20-80% ethyl acetate in hexanes) to give title compound (159 mg, 51% yield) as a white solid. ¹H NMR (400 MHz, CHLOROFORM-d) δ ppm 8.37 (s, 1H), 8.25 (d, J=8.21 Hz, 2H), 7.72 (d, J=8.4 Hz, 2H), 7.63 (t, J=8.1 Hz, 1H), 7.54 (t, J=7.6 Hz, 2H), 7.47 (d, J=8.5 Hz, 2H), 6.96 (s, 1H), 6.28 (s, 1H), 4.61 (d, J=12.4 Hz, 2H), 3.40 (t, J=6.3 Hz, 2H), 3.09 (t, J=13.3 Hz, 2H), 2.03 (m, 1H), 1.91 (d, J=13.3 Hz, 2H), 1.36 (s, 9H).

### Step 4: N-{1-[7-Benzenesulfonyl-6-(1-methyl-1H-pyrazol-4-yl)-7H-pyrrolo[2,3-d]pyrimidin-4-yl]-piperidin-4-ylmethyl}-4-tert-butyl-benzamide

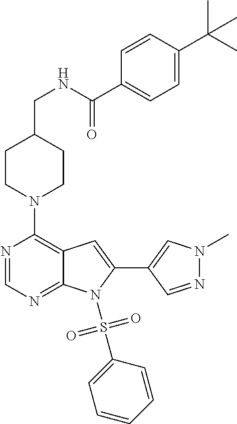 [SMILES: Cn1cc(-c2cc3c(N4CCC(CNC(=O)c5ccc(C(C)(C)C)cc5)CC4)ncnc3n2S(=O)(=O)c2ccccc2)cn1]

To a mixture of N-[1-(7-Benzenesulfonyl-6-iodo-7H-pyrrolo[2,3-d]pyrimidin-4-yl)-piperidin-4-ylmethyl]-4-tert-butyl-benzamide (159 mg, 0.242 mmol, Eq: 1.00), 1-methyl-1H-pyrazol-4-ylboronic acid (36.5 mg, 0.290 mmol, Eq: 1.2), Pd(PPh₃)₄ (28 mg, 0.024 mmol, Eq: 0.1) and potassium carbonate (100 mg, 0.725 mmol, Eq: 3.00) was added DME (1.29 ml)/Water (322 μl) and heated in the microwave to 150° C. for 1 h. The reaction mixture was diluted with DCM and washed with water. The combined organic phases were dried over anhydrous sodium sulfate then the solvent was removed under reduced pressure. The crude material was purified by column chromatography (silica, 30-100% ethyl acetate in hexanes) to give title compound (85 mg, 58% yield) as a white solid. LC/MS: m/z calculated for C₃₃H₃₇N₇O₃S([M+H]⁺): 612.7 Found: 612.4

### Step 5: 4-tert-Butyl-N-{1-[6-(1-methyl-1H-pyrazol-4-yl)-7H-pyrrolo[2,3-d]pyrimidin-4-yl]-piperidin-4-ylmethyl}-benzamide

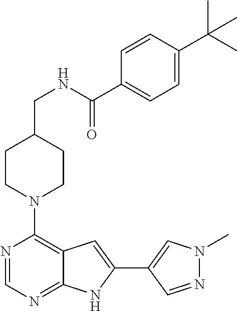 [SMILES: Cn1cc(-c2cc3c(N4CCC(CNC(=O)c5ccc(C(C)(C)C)cc5)CC4)ncnc3[nH]2)cn1]

To a solution of N-{1-[7-Benzenesulfonyl-6-(1-methyl-1H-pyrazol-4-yl)-7H-pyrrolo[2,3-d]pyrimidin-4-yl]-piperidin-4-ylmethyl}-4-tert-butyl-benzamide (85 mg, 0.139 mmol, Eq: 1.00) in THF (926 μl)/MeOH (463 μl) was added cesium carbonate (136 mg, 0.417 mmol, Eq: 3.00) and stirred at r.t. for 16 h. The reaction mixture was purified by column chromatography (silica, 1-6% methanol in DCM containing NH₄OH) to give title compound (45 mg, 68.7% yield) as a white solid. ¹H NMR (400 MHz, DMSO-d₆) δ ppm 11.95 (s, 1H), 8.45 (t, J=5.4 Hz, 1H), 8.11 (s, 1H), 7.93 (s, 1H), 7.80 (d, J=7.9 Hz, 2H), 7.49 (d, J=7.9 Hz, 2H), 6.75 (s, 1H), 4.71 (d, J=13.8 Hz, 2H), 3.88 (s, 3H), 3.20 (t, J=6.2 Hz, 2H), 3.06 (t, J=12.8 Hz, 2H), 1.97 (m, 1H), 1.82 (d, J=13.3 Hz, 2H), 1.31 (s, 9H), 1.23 (d, J=14.3 Hz, 2H); LC/MS: m/z calculated for C₂₇H₃₃N₇O ([M+H]⁺): 472.6 Found: 472.4

### Example I-21

### 4-tert-Butyl-N-{4-[6-(1-methyl-1H-pyrazol-4-yl)-7H-pyrrolo[2,3-d]pyrimidin-4-yl]-benzyl}-benzamide

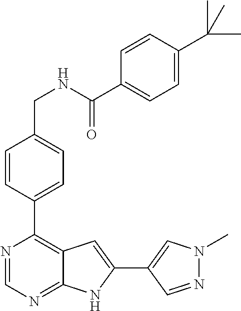 [SMILES: Cn1cc(-c2cc3c(-c4ccc(CNC(=O)c5ccc(C(C)(C)C)cc5)cc4)ncnc3[nH]2)cn1]

### Step 1: 4-[7-(Toluene-4-sulfonyl)-7H-pyrrolo[2,3-d]pyrimidin-4-yl]-benzyl}-carbamic acid tert-butyl ester

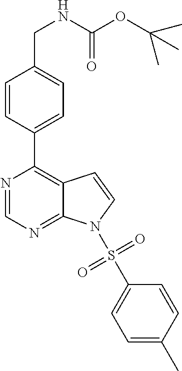 [SMILES: Cc1ccc(S(=O)(=O)n2ccc3c(-c4ccc(CNC(=O)OC(C)(C)C)cc4)ncnc32)cc1]

In a 20 mL sealable microwave tube, 4-chloro-7-tosyl-7H-pyrrolo[2,3-d]pyrimidine (1 g, 3.25 mmol, Eq: 1.00), 4-((tert-butoxycarbonylamino)methyl)phenylboronic acid (1.22 g, 4.87 mmol, Eq: 1.5) and potassium carbonate (1.8 g, 13.0 mmol, Eq: 4.00) in 5 mL of water were combined with DME (10 mL). Pd(PPh₃)₄ (375 mg, 0.325 mmol, Eq: 0.1) was added. The reaction mixture was heated in a microwave at 150° C. for 30 minutes. The solution was washed with EtOAc and brine. The combined organic phases were dried over anhydrous sodium sulfate then the solvent was removed under reduced pressure. The crude material was purified by column chromatography (silica, 5-70% ethyl acetate in hexanes). The title compound was obtained as a white solid (700 mg, 45% yield). LC/MS: m/z calculated for C₂₅H₂₆N₄O₄S([M+H]⁺): 479.5 Found: 479.3

### Step 2: 4-[6-Bromo-7-(toluene-4-sulfonyl)-7H-pyrrolo[2,3-d]pyrimidin-4-yl]-benzyl}-carbamic acid tert-butyl ester

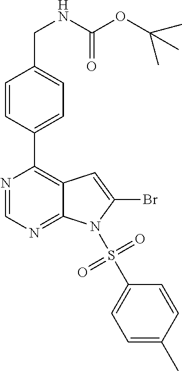 [SMILES: Cc1ccc(S(=O)(=O)n2c(Br)cc3c(-c4ccc(CNC(=O)OC(C)(C)C)cc4)ncnc32)cc1]

In a 100 mL round-bottomed flask, 4-[7-(toluene-4-sulfonyl)-7H-pyrrolo[2,3-d]pyrimidin-4-yl]-benzyl}-carbamic acid tert-butyl ester (500 mg, 1.04 mmol, Eq: 1.00) was dissolved in THF (10 mL) and cooled down to −78° C. LDA 2M solution in heptanes/THF/ethylbenzene (1.31 mL, 2.61 mmol, Eq: 2.5) was added at −78 C under a nitrogen atmosphere to give a dark brown solution. The reaction mixture was stirred at −78° C. for 1 hr 30 minutes. 1,2-dibromo-1,1,2,2-tetrachloroethane (851 mg, 2.61 mmol, Eq: 2.5) in 5 mL THF was added and the reaction mixture was stirred at −78 C for 2 hours. Water was added. The reaction mixture was diluted with EtOAc and washed with brine. The combined organic phases were dried over anhydrous sodium sulfate then evaporated. The crude material was purified by column chromatography (silica, 5-70% ethyl acetate in hexanes). The title compound was obtained as a white solid (450 mg, 77% yield). LC/MS: m/z calculated for C₂₅H₂₅BrN₄O₄S(M+H⁺): 558.4 Found: 558.8

### Step 3: {4-[6-(1-Methyl-1H-pyrazol-4-yl)-7H-pyrrolo[2,3-d]pyrimidin-4-yl]-benzyl}-carbamic acid tert-butyl ester

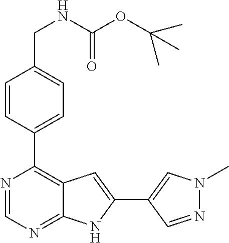 [SMILES: Cn1cc(-c2cc3c(-c4ccc(CNC(=O)OC(C)(C)C)cc4)ncnc3[nH]2)cn1]

In a 10 mL sealable microwave tube, 4-[6-Bromo-7-(toluene-4-sulfonyl)-7H-pyrrolo[2,3-d]pyrimidin-4-yl]-benzyl}-carbamic acid tert-butyl ester (200 mg, 0.359 mmol, Eq: 1.00), 1-methyl-4-(4,4,5,5-tetramethyl-1,3,2-dioxaborolan-2-yl)-1H-pyrazole (192 mg, 0.923 mmol, Eq: 2.57) and potassium carbonate (198 mg, 1.44 mmol, Eq: 4.00) in water (1 mL) were combined with DME (4 mL). Pd(PPh₃)₄ (42 mg, 0.036 mmol, Eq: 0.1) was added and the reaction mixture was sealed and heated in a microwave at 150° C. for 60 minutes. The reaction mixture was diluted with EtOAc then washed with brine. The combined organic phases were dried over anhydrous sodium sulfate. The solvent was removed under reduced pressure. The crude material was triturated with DCM then filtered to afford the title compound as a yellow solid (32 mg, 22% yield). LC/MS: m/z calculated for C₂₂H₂₄N₆O₂([M+H]⁺): 405.4 Found: 405.2

### Step 4: 4-tert-Butyl-N-{4-[6-(1-methyl-1H-pyrazol-4-yl)-7H-pyrrolo[2,3-d]pyrimidin-4-yl]-benzyl}-benzamide

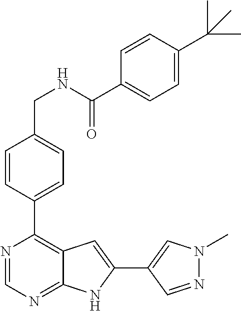 [SMILES: Cn1cc(-c2cc3c(-c4ccc(CNC(=O)c5ccc(C(C)(C)C)cc5)cc4)ncnc3[nH]2)cn1]

In a 20 mL scintillation vial, {4-[6-(1-methyl-1H-pyrazol-4-yl)-7H-pyrrolo[2,3-d]pyrimidin-4-yl]-benzyl}-carbamic acid tert-butyl ester (30 mg, 0.074 mmol, Eq: 1.00) was combined with 1 mL DCM and 1 mL TFA. The solution was stirred at room temperature for 1 hour. The solvent was removed under reduced pressure. The crude material was dissolved in DMF (2 mL). 4-tert-butylbenzoic acid (14.5 mg, 0.082 mmol, Eq: 1.1), DIPEA (0.052 mL, 0.297 mmol, Eq: 4.00) and HATU (31.0 mg, 0.082 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was triturated with DCM and the resulting solid was filtered. The title compound was obtained as a solid (11 mg, 32% yield). LC/MS: m/z calculated for C₂₈H₂₈N₆O ([M+H]⁺): 465.5 Found: 465.2

### Example I-22

### 4-Cyclopropyl-N-{4-[6-(1-methyl-1H-pyrazol-4-yl)-7H-pyrrolo[2,3-d]pyrimidin-4-yl]-benzyl}-benzamide

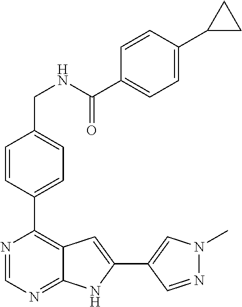 [SMILES: Cn1cc(-c2cc3c(-c4ccc(CNC(=O)c5ccc(C6CC6)cc5)cc4)ncnc3[nH]2)cn1]

Following a similar procedure described in example 21, step 4, using 4-cyclopropylbenzoic acid (12.0 mg, 0.074 mmol, Eq: 1.00), the title compound was obtained as a solid (16 mg, 48% yield). LC/MS: m/z calculated for C₂₇H₂₄N₆O([M+H]⁺): 449.5 Found: 449.2

### Example I-23

### 4-Isopropyl-N-{4-[6-(1-methyl-1H-pyrazol-4-yl)-7H-pyrrolo[2,3-d]pyrimidin-4-yl]-benzyl}-benzamide

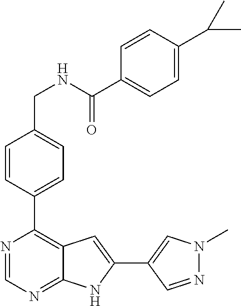 [SMILES: CC(C)c1ccc(C(=O)NCc2ccc(-c3ncnc4[nH]c(-c5cnn(C)c5)cc34)cc2)cc1]

Following a similar procedure described in example 21, step 4, using 4-isopropylbenzoic acid (13.4 mg, 0.082 mmol, Eq: 1.1), the title compound was obtained as a solid (5 mg, 15% yield). LC/MS: m/z calculated for C₂₇H₂₆N₆O([M+H]⁺): 451.5 Found: 451.3

### Example I-24

### N-{4-[6-(1-Methyl-1H-pyrazol-4-yl)-7H-pyrrolo[2,3-d]pyrimidin-4-yl]-benzyl}-4-oxetan-3-yl-benzamide

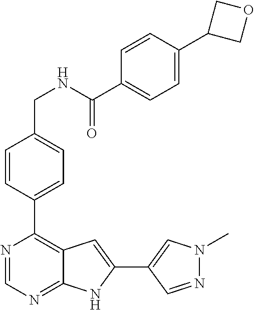 [SMILES: Cn1cc(-c2cc3c(-c4ccc(CNC(=O)c5ccc(C6COC6)cc5)cc4)ncnc3[nH]2)cn1]

### Step 1: 4-Oxetan-3-yl-benzoic acid methyl ester

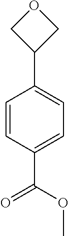

In a 20 mL sealable microwave vial, 4-(methoxycarbonyl)phenylboronic acid (978 mg, 5.44 mmol, Eq: 2.0), trand-2-aminocyclohexanol hydrochloride (50 mg, 0.326 mmol, Eq: 0.12) and nickel iodide (102 mg, 0.326 mmol, Eq: 0.12) were combined with isopropanol (8 mL) to give a white suspension. NaHMDS (997 mg, 5.44 mmol, Eq: 2.0) was added. The reaction mixture was back-filled with argon and stirred for five minutes. 3-iodooxetane (0.5 g, 2.72 mmol, Eq: 1.00) was added. The reaction mixture was sealed and then heated at 80° C. for 20 minutes in the microwave. After this time, TLC showed two possible product spots, close together in Rf. The reaction mixture was diluted with isopropanol, then filtered through filter paper. The solvent was concentrated to give a yellow oil. This product was dissolved in methylene chloride and the solution was concentrated over silica gel. The silica-gel supported crude product was loaded onto a 40 gram silica gel column. Flash chromatography (5% ethyl acetate-hexanes ramped to 10% ethyl acetate-hexanes). The title compound was isolated as an oil (118 mg, 23% yield). The other side product isolated corresponded to the 4-Oxetan-3-yl-benzoic acid isopropyl ester (77 mg, 13% yield).

### Step 2: 4-Oxetan-3-yl-benzoic acid

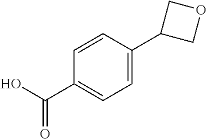 [SMILES: O=C(O)c1ccc(C2COC2)cc1]

In a 100 mL pear-shaped flask, methyl 4-(oxetan-3-yl)benzoate (118 mg, 0.614 mmol, Eq: 1.00) and lithium hydroxide monohydrate (40 mg, 0.953 mmol, Eq: 1.55) were combined with THF (2.5 mL) to give a colorless solution. Water (2.5 mL) was added. The reaction mixture was stirred overnight at room temperature. In the morning, TLC showed a small amount of remaining starting material. An additional 40 mg of LiOH monohydrate was added, and the reaction mixture was stirred overnight at room temperature again. The solvent was removed under reduced pressure. The aqueous residue was then diluted with 10 mL water and this solution was extracted with 20 mL of 1:1 hexane-ethyl acetate. The aqueous phase was then acidified with several drops of 4 N aqueous HCl, giving a white suspension. This suspension was extracted with ethyl acetate. The organic extracts were dried over anhydrous sodium sulfate and the solvent was removed under reduced pressure. The title compound was obtained as a white solid (88 mg, 80% yield). The product was used as is without further purification.

### Step 3: N-{4-[6-(1-Methyl-1H-pyrazol-4-yl)-7H-pyrrolo[2,3-d]pyrimidin-4-yl]-benzyl}-4-oxetan-3-yl-benzamide

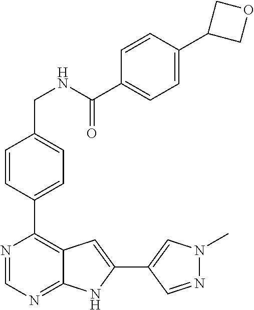 [SMILES: Cn1cc(-c2cc3c(-c4ccc(CNC(=O)c5ccc(C6COC6)cc5)cc4)ncnc3[nH]2)cn1]

In a 20 mL scintillation vial, {4-[6-(1-Methyl-1H-pyrazol-4-yl)-7H-pyrrolo[2,3-d]pyrimidin-4-yl]-benzyl}-carbamic acid tert-butyl ester (40 mg, 0.099 mmol, Eq: 1.00) was combined with 3 mL DCM and 3 mL TFA. The solution was stirred at room temperature for 1 hour. The solvent was removed under reduced pressure. The crude material was dissolved in DMF (4 mL). 4-Oxetan-3-yl-benzoic acid (18 mg, 0.099 mmol, Eq: 1.00), DIPEA (0.069 mL, 0.396 mmol, Eq: 4.00) and HATU (38 mg, 0.099 mmol, Eq: 1.00) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was triturated with DCM and the resulting solid was filtered. The title compound was obtained as a solid (3 mg, 7% yield). LC/MS: m/z calculated for C₂₇H₂₄N₆O₂([M+H]⁺): 465.5 Found: 465.2

### Example I-25

### 4-(3-Methyl-oxetan-3-yl)-N-{4-[6-(1-methyl-1H-pyrazol-4-yl)-7H-pyrrolo[2,3-d]pyrimidin-4-yl]-benzyl}-benzamide

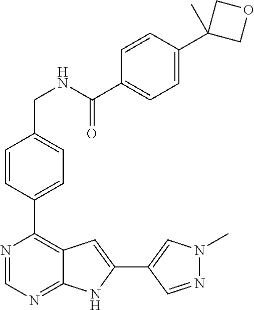 [SMILES: Cn1cc(-c2cc3c(-c4ccc(CNC(=O)c5ccc(C6(C)COC6)cc5)cc4)ncnc3[nH]2)cn1]

### Step 1: 4-(3-Methyl-oxetan-3-yl)-benzoic acid

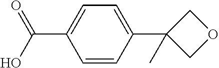

In a 250 mL three-necked flask, 3-(4-bromophenyl)-3-methyloxetane (1.05 g, 4.62 mmol) was combined with THF (35 mL) to give a colorless solution. This solution was cooled to −78° C. in a dry ice-acetone bath. To the cold solution was added drop wise a 1.6 M solution of nBuLi in hexanes (3.32 mL, 5.32 mmol). Drop wise addition occurred over the course of 10 minutes. The reaction mixture was stirred at −78° C. for 1 hour. After this time, carbon dioxide gas, which was generated in a separate flask from dry ice, was added to the reaction mixture via a long needle. The reaction mixture quickly turned to light yellow. Carbon dioxide was bubbled in at low temperature for another 20 minutes. After this time, the reaction mixture was a white suspension. The reaction mixture was warmed to room temperature then slowly quenched with water. The organic solvent was evaporated off. The resulting mixture was extracted with a 1:1 solution of ethyl acetate and hexanes. The aqueous phase was then brought to acidic pH though the addition of 4 N aqueous HCl. The resulting white suspension was vacuum filtered using a Büchner funnel. The collected white solids were further dried down on the vacuum funnel and then further dried in the vacuum oven, giving 4-(3-methyl-oxetan-3-yl)-benzoic acid (456 mg, 51%). ¹H NMR (300 MHz, DMSO-d₆) δ ppm 12.89 (br. s., 1H), 7.93 (d, J=8.48 Hz, 2H), 7.36 (d, J=8.67 Hz, 2H), 4.81 (d, J=5.84 Hz, 2H), 4.56 (d, J=6.03 Hz, 2H), 1.64 (s, 3H).

### Step 2: 4-(3-Methyl-oxetan-3-yl)-N-{4-[6-(1-methyl-1H-pyrazol-4-yl)-7H-pyrrolo[2,3-d]pyrimidin-4-yl]-benzyl}-benzamide

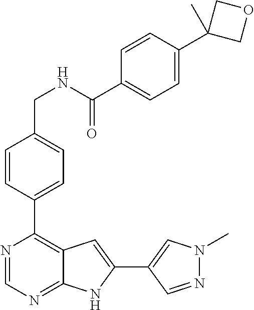 [SMILES: Cn1cc(-c2cc3c(-c4ccc(CNC(=O)c5ccc(C6(C)COC6)cc5)cc4)ncnc3[nH]2)cn1]

In a 20 mL scintillation vial, {4-[6-(1-methyl-1H-pyrazol-4-yl)-7H-pyrrolo[2,3-d]pyrimidin-4-yl]-benzyl}-carbamic acid tert-butyl ester (60 mg, 0.148 mmol, Eq: 1.00) was combined with 3 mL DCM and 3 mL TFA. The solution was stirred at room temperature for 1 hour. The solvent was removed under reduced pressure. The crude material was dissolved in DMF (4 mL). 4-Oxetan-3-yl-benzoic acid (31 mg, 0.163 mmol, Eq: 1.1), DIPEA (0.104 mL, 0.593 mmol, Eq: 4.00) and HATU (62 mg, 0.163 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was triturated with DCM and the resulting solid was filtered. The title compound was obtained as a yellow solid (15 mg, 21% yield). LC/MS: m/z calculated for C₂₈H₂₆N₆O₂([M+H]⁺): 479.5 Found: 479.2

### Example I-26

### 4,5,6,7-Tetrahydro-benzo[b]thiophene-2-carboxylic acid 4-[6-(1-methyl-1H-pyrazol-4-yl)-7H-pyrrolo[2,3-d]pyrimidin-4-yl]-benzylamide

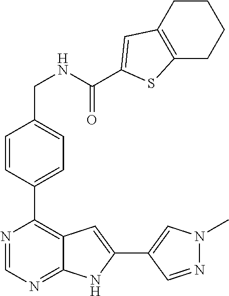 [SMILES: Cn1cc(-c2cc3c(-c4ccc(CNC(=O)c5cc6c(s5)CCCC6)cc4)ncnc3[nH]2)cn1]

In a 20 mL scintillation vial, {4-[6-(1-methyl-1H-pyrazol-4-yl)-7H-pyrrolo[2,3-d]pyrimidin-4-yl]-benzyl}-carbamic acid tert-butyl ester (40 mg, 0.099 mmol, Eq: 1.00) was combined with 3 mL DCM and 3 mL TFA. The solution was stirred at room temperature for 1 hour. The solvent was removed under reduced pressure. The crude material was dissolved in DMF (4 mL). 4,5,6,7-Tetrahydro-benzo[b]thiophene-2-carboxylic acid (20 mg, 0.109 mmol, Eq: 1.1), DIPEA (0.069 mL, 0.396 mmol, Eq: 4.00) and HATU (41 mg, 0.109 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The crude material was purified by column chromatography (silica, 0-10% MeOH in DCM). The title compound was obtained as a solid (17 mg, 37% yield). LC/MS: m/z calculated for C₂₆H₂₄N₆OS([M+H]⁺): 469.5 Found: 469.2

### Example I-27

### 4-tert-Butyl-N-{2-fluoro-4-[6-(1-methyl-1H-pyrazol-4-yl)-7H-pyrrolo[2,3-d]pyrimidin-4-yl]-benzyl}-benzamide

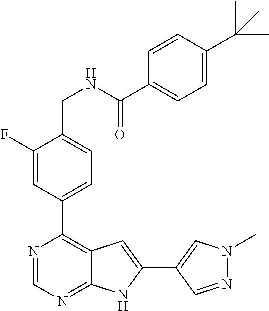 [SMILES: Cn1cc(-c2cc3c(-c4ccc(CNC(=O)c5ccc(C(C)(C)C)cc5)c(F)c4)ncnc3[nH]2)cn1]

### Step 1: {2-Fluoro-4-[7-(toluene-4-sulfonyl)-7H-pyrrolo[2,3-d]pyrimidin-4-yl]-benzyl}-carbamic acid tert-butyl ester

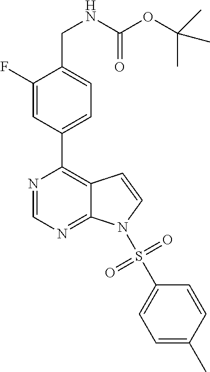 [SMILES: Cc1ccc(S(=O)(=O)n2ccc3c(-c4ccc(CNC(=O)OC(C)(C)C)c(F)c4)ncnc32)cc1]

In a 20 mL sealable microwave tube, 4-chloro-7-tosyl-7H-pyrrolo[2,3-d]pyrimidine (1 g, 3.25 mmol, Eq: 1.00), [2-Fluoro-4-(4,4,5,5-tetramethyl-[1,3,2]dioxaborolan-2-yl)-benzyl]-carbamic acid tert-butyl ester (1.14 g, 3.25 mmol, Eq: 1.00) and potassium carbonate (1.8 g, 13.0 mmol, Eq: 4.00) in 5 mL of water were combined with DME (10 mL). Pd(PPh₃)₄ (375 mg, 0.325 mmol, Eq: 0.1) was added. The reaction mixture was heated in a microwave at 150° C. for 30 minutes. The solution was washed with EtOAc and brine. The combined organic phases were dried over anhydrous sodium sulfate then the solvent was removed under reduced pressure. The crude material was purified by column chromatography (silica, 5-70% ethyl acetate in hexanes). The title compound was obtained as a white solid (950 mg, 59% yield). LC/MS: m/z calculated for C₂₅H₂₅FN₄O₄S([M+H]⁺): 497.5 Found: 497.2

### Step 2: {4-[6-Bromo-7-(toluene-4-sulfonyl)-7H-pyrrolo[2,3-d]pyrimidin-4-yl]-2-fluoro-benzyl}-carbamic acid tert-butyl ester

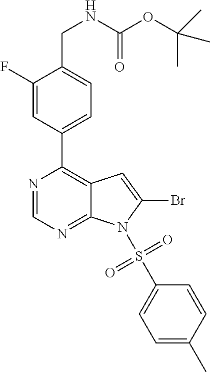 [SMILES: Cc1ccc(S(=O)(=O)n2c(Br)cc3c(-c4ccc(CNC(=O)OC(C)(C)C)c(F)c4)ncnc32)cc1]

In a 100 mL round-bottomed flask, {2-Fluoro-4-[7-(toluene-4-sulfonyl)-7H-pyrrolo[2,3-d]pyrimidin-4-yl]-benzyl}-carbamic acid tert-butyl ester (2 g, 4.03 mmol, Eq: 1.00) was dissolved in THF (40 mL) and cooled down to −78° C. LDA 2M solution in heptanes/THF/ethylbenzene (5.03 mL, 10.1 mmol, Eq: 2.5) was added at −78 C under a nitrogen atmosphere to give a dark brown solution. The reaction mixture was stirred at −78° C. for 1 hr 30 minutes. 1,2-dibromo-1,1,2,2-tetrachloroethane (3.28 g, 10.1 mmol, Eq: 2.5) in 10 mL THF was added and the reaction mixture was stirred at −78 C for 2 hours. Brine was added. The reaction mixture was diluted with EtOAc and washed with brine. The combined organic phases were dried over anhydrous sodium sulfate then evaporated. The crude material was purified by column chromatography (silica, 5-70% ethyl acetate in hexanes). The title compound was obtained as a beige solid (1.2 g, 52% yield). LC/MS: m/z calculated for C₂₅H₂₄BrFN₄O₄S([M+H]⁺): 576.4 Found: 577.1

### Step 3: {2-Fluoro-4-[6-(1-methyl-1H-pyrazol-4-yl)-7H-pyrrolo[2,3-d]pyrimidin-4-yl]-benzyl}-carbamic acid tert-butyl ester

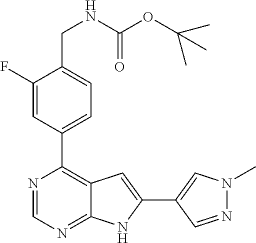 [SMILES: Cn1cc(-c2cc3c(-c4ccc(CNC(=O)OC(C)(C)C)c(F)c4)ncnc3[nH]2)cn1]

In a 20 mL sealable microwave tube, {4-[6-Bromo-7-(toluene-4-sulfonyl)-7H-pyrrolo[2,3-d]pyrimidin-4-yl]-2-fluoro-benzyl}-carbamic acid tert-butyl ester (400 mg, 0.695 mmol, Eq: 1.00), 1-methyl-4-(4,4,5,5-tetramethyl-1,3,2-dioxaborolan-2-yl)-1H-pyrazole (226 mg, 1.09 mmol, Eq: 1.6) and potassium carbonate (384 mg, 2.78 mmol, Eq: 4.00) in water (3 mL) were combined with DME (6 mL). Pd(PPh₃)₄ (80 mg, 0.069 mmol, Eq: 0.1) was added and the reaction mixture was sealed and heated in a microwave at 160° C. for 60 minutes. The reaction mixture was diluted with EtOAc then washed with brine. The combined organic phases were dried over anhydrous sodium sulfate. The solvent was removed under reduced pressure. The crude material was triturated with DCM then filtered to afford the title compound as a brown solid (75 mg, 26% yield). LC/MS: m/z calculated for C₂₂H₂₃FN₆O₂([M+H]⁺): 423.4 Found: 423.3

### Step 4: 4-tert-Butyl-N-{2-fluoro-4-[6-(1-methyl-1H-pyrazol-4-yl)-7H-pyrrolo[2,3-d]pyrimidin-4-yl]-benzyl}-benzamide

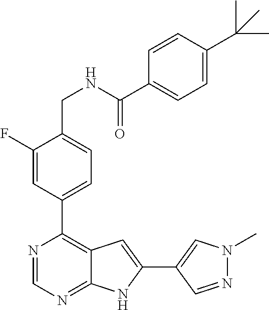 [SMILES: Cn1cc(-c2cc3c(-c4ccc(CNC(=O)c5ccc(C(C)(C)C)cc5)c(F)c4)ncnc3[nH]2)cn1]

In a 20 mL scintillation vial, {2-Fluoro-4-[6-(1-methyl-1H-pyrazol-4-yl)-7H-pyrrolo[2,3-d]pyrimidin-4-yl]-benzyl}-carbamic acid tert-butyl ester (40 mg, 0.094 mmol, Eq: 1.00) was combined with 1 mL DCM and 1 mL TFA. The solution was stirred at room temperature for 1 hour. The solvent was removed under reduced pressure. The crude material was dissolved in DMF (2 mL). 4-tert-butylbenzoic acid (19 mg, 0.104 mmol, Eq: 1.1), DIPEA (0.066 mL, 0.379 mmol, Eq: 4.00) and HATU (40 mg, 0.104 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was triturated with DCM and the resulting solid was filtered. The title compound was obtained as a solid (17 mg, 37% yield). LC/MS: m/z calculated for C₂₈H₂₇FN₆O([M+H]⁺): 483.5 Found: 483.2

### Example I-28

### 6-tert-Butyl-N-{2-fluoro-4-[6-(1-methyl-1H-pyrazol-4-yl)-7H-pyrrolo[2,3-d]pyrimidin-4-yl]-benzyl}-nicotinamide

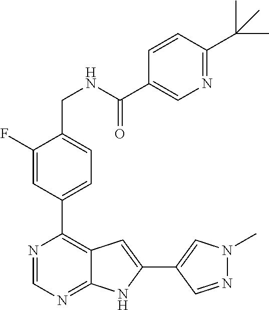 [SMILES: Cn1cc(-c2cc3c(-c4ccc(CNC(=O)c5ccc(C(C)(C)C)nc5)c(F)c4)ncnc3[nH]2)cn1]

### Step 1: 6-tert-Butyl-nicotinic acid

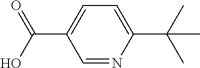

To a suspension of nicotinic acid (2 g, 16 mmol, Eq: 1.00) in water was added concentrated sulfuric acid (1 mL, 18.8 mmol, Eq: 1.2) and the mixture was stirred under nitrogen to form a clear solution. Pivalic acid (1.83 g, 17.9 mmol, Eq: 1.1) was added and stirring under argon at ambient temperature continued for 10 minutes. Silver nitrate (125 mg, 0.736 mmol) was added followed by ammonium persulfate (295 mg, 1.29 mmol, Eq: 0.08), the flask wrapped in aluminum foil to exclude light and the mixture heated to 90° C. under nitrogen. The reaction mixture was cooled to ambient temperature after 2 hours and left to stand overnight. Extraction of the reaction mixture with ethyl acetate failed to yield any significant amount of the expected product. LC/MS did indicate the presence of the product in the aqueous layer. The aqueous mixture was concentrated in vacuo to a colorless solid. The solid was triturated with THF, filtered and the filtrate was concentrated in vacuo. The residue was re-triturated with methanol, filtered and then the filtrate was concentrated in vacuo. The concentrated filtrates were purified by reverse phase chromatography using a 85 g C-18 column with gradient elution from 10% acetonitrile in water to 100% acetonitrile. The fractions containing the desired product were combined and concentrated to a colorless aqueous suspension (˜5 mL volume). Additional water (˜20 mL) was added to form a clear solution and the mixture was lyophilized to provide title compound as a colorless, amorphous lyophilized solid (139 mg, 5% yield). LC/MS: m/z calculated for C₁₀H₁₄NO₂[(M+H)⁺]: 180.2, 483.5 Found: 180.1

### Step 2: 6-tert-Butyl-N-{2-fluoro-4-[6-(1-methyl-1H-pyrazol-4-yl)-7H-pyrrolo[2,3-d]pyrimidin-4-yl]-benzyl}-nicotinamide

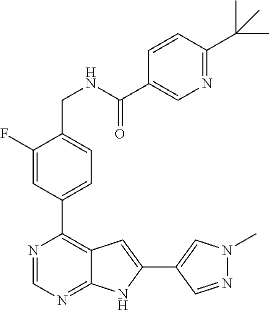 [SMILES: Cn1cc(-c2cc3c(-c4ccc(CNC(=O)c5ccc(C(C)(C)C)nc5)c(F)c4)ncnc3[nH]2)cn1]

In a 20 mL scintillation vial, {2-Fluoro-4-[6-(1-methyl-1H-pyrazol-4-yl)-7H-pyrrolo[2,3-d]pyrimidin-4-yl]-benzyl}-carbamic acid tert-butyl ester (56 mg, 0.133 mmol, Eq: 1.00) was combined with 2 mL DCM and 2 mL TFA. The solution was stirred at room temperature for 30 minutes. The solvent was removed under reduced pressure. The crude material was dissolved in DMF (2 mL). 6-tert-Butyl-nicotinic acid (40 mg, 0.233 mmol, Eq: 1.7), DIPEA (0.093 mL, 0.53 mmol, Eq: 4.00) and HATU (55 mg, 0.146 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was triturated with DCM and was filtered. The title compound was obtained as a yellow solid (40 mg, 62% yield). LC/MS: m/z calculated for C₂₇H₂₆FN₇O([M+H]⁺): 484.5 Found: 483.3

### Example I-29

### 5-Methyl-thiophene-2-carboxylic acid 2-fluoro-4-[6-(1-methyl-1H-pyrazol-4-yl)-7H-pyrrolo[2,3-d]pyrimidin-4-yl]-benzylamide

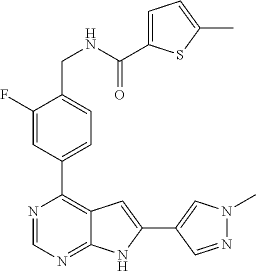 [SMILES: Cc1ccc(C(=O)NCc2ccc(-c3ncnc4[nH]c(-c5cnn(C)c5)cc34)cc2F)s1]

Following a similar procedure described in example 28, step 2, using 5-methylthiophene-2-carboxylic acid (22 mg, 0.156 mmol, Eq: 1.1), the title compound was obtained as a solid (42 mg, 66% yield). LC/MS: m/z calculated for C₂₃H₁₉FN₆OS([M+H]⁺): 447.5 Found: 447.2

### Example I-30

### 4-tert-Butyl-N-(2-fluoro-4-{6-[1-(2-hydroxy-ethyl)-1H-pyrazol-4-yl]-7H-pyrrolo[2,3-d]pyrimidin-4-yl}-benzyl)-benzamide

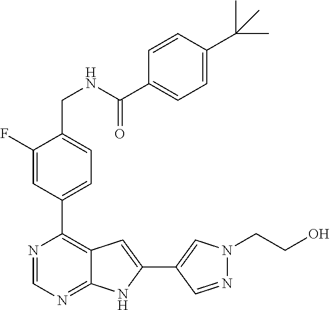 [SMILES: CC(C)(C)c1ccc(C(=O)NCc2ccc(-c3ncnc4[nH]c(-c5cnn(CCO)c5)cc34)cc2F)cc1]

### Step 1: N-[4-(6-Bromo-7H-pyrrolo[2,3-d]pyrimidin-4-yl)-2-fluoro-benzyl]-4-tert-butyl-benzamide

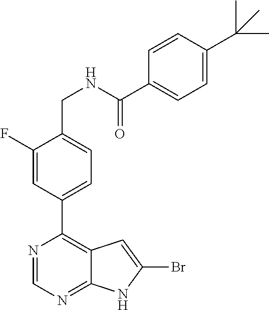 [SMILES: CC(C)(C)c1ccc(C(=O)NCc2ccc(-c3ncnc4[nH]c(Br)cc34)cc2F)cc1]

In a 20 mL scintillation vial, {4-[6-Bromo-7-(toluene-4-sulfonyl)-7H-pyrrolo[2,3-d]pyrimidin-4-yl]-2-fluoro-benzyl}-carbamic acid tert-butyl ester (200 mg, 0.348 mmol, Eq: 1.00) was combined with 1 mL DCM and 1 mL TFA. The solution was stirred at room temperature for 30 minutes. The solvent was removed under reduced pressure. The crude material was dissolved in DMF (2 mL). 4-tert-Butyl-benzoic acid (68 mg, 0.382 mmol, Eq: 1.1), DIPEA (0.243 mL, 1.39 mmol, Eq: 4.00) and HATU (145 mg, 0.382 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The crude material was stored at room temperature overnight. The resulting solid was triturated with DCM and was filtered. The title compound was obtained as a solid (96 mg, 57% yield). LC/MS: m/z calculated for C₂₄H₂₂BrFN₄O([M+H]⁺): 482.3 Found: 483.0

### Step 2: 4-tert-Butyl-N-(2-fluoro-4-{6-[1-(2-hydroxy-ethyl)-1H-pyrazol-4-yl]-7H-pyrrolo[2,3-d]pyrimidin-4-yl}-benzyl)-benzamide

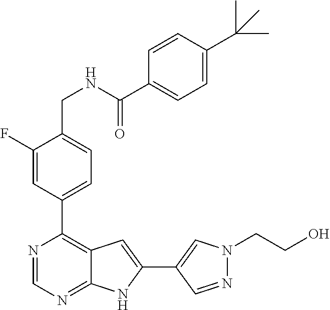 [SMILES: CC(C)(C)c1ccc(C(=O)NCc2ccc(-c3ncnc4[nH]c(-c5cnn(CCO)c5)cc34)cc2F)cc1]

In a 20 mL sealable microwave tube, N-[4-(6-Bromo-7H-pyrrolo[2,3-d]pyrimidin-4-yl)-2-fluoro-benzyl]-4-tert-butyl-benzamide (90 mg, 0.187 mmol, Eq: 1.00), 2-(4-(4,4,5,5-tetramethyl-1,3,2-dioxaborolan-2-yl)-1H-pyrazol-1-yl)ethanol (49.0 mg, 0.206 mmol, Eq: 1.1) and potassium carbonate (103 mg, 0.748 mmol, Eq: 4.00) in water (1 mL) were combined with DME (4 mL). Pd(PPh₃)₄ (22 mg, 0.019 mmol, Eq: 0.1) was added and the reaction mixture was sealed and heated in a microwave at 150° C. for 30 minutes. The reaction mixture was diluted with EtOAc then washed with brine. The combined organic phases were dried over anhydrous sodium sulfate. The solvent was removed under reduced pressure. The crude material was triturated with DCM then filtered to afford the title compound as a brown solid (22 mg, 23% yield). LC/MS: m/z calculated for C₂₉H₂₉FN₆O₂([M+H]⁺): 513.5 Found: 513.2

### Example I-31

### 4-tert-Butyl-N-{2-fluoro-4-[6-(1-methyl-1H-pyrazol-4-yl)-7H-pyrrolo[2,3-d]pyrimidin-4-yl]-benzyl}-N-methyl-benzamide

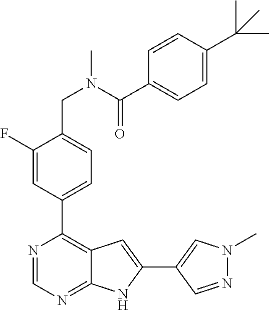 [SMILES: CN(Cc1ccc(-c2ncnc3[nH]c(-c4cnn(C)c4)cc23)cc1F)C(=O)c1ccc(C(C)(C)C)cc1]

### Step 1: (4-Bromo-2-fluoro-benzyl)-methyl-carbamic acid tert-butyl ester

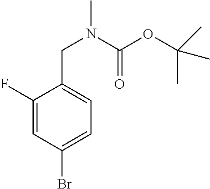 [SMILES: CN(Cc1ccc(Br)cc1F)C(=O)OC(C)(C)C]

In a 100 mL round-bottomed flask, tert-butyl 4-bromo-2-fluorobenzylcarbamate (2 g, 6.58 mmol, Eq: 1.00), methyl iodide (0.7 mL, 11.2 mmol, Eq: 1.7) and NaH in 60% oil dispersion (395 mg, 16.5 mmol, Eq: 2.5) were combined with DMF (40 mL) at 0° C. The reaction mixture was stirred and let warmed to room temperature for 3 hours. The reaction was quenched with MeOH. The reaction mixture was diluted with EtOAc and washed with brine. The combined organic phases were dried over anhydrous sodium sulfate then the solvent was removed under reduced pressure. The crude material was purified by column chromatography (silica, 5-40% ethyl acetate in hexanes). The title compound was obtained as an oil (1.8 g, 86% yield).

### Step 2: [2-Fluoro-4-(4,4,5,5-tetramethyl-[1,3,2]dioxaborolan-2-yl)-benzyl]-methyl-carbamic acid tert-butyl ester

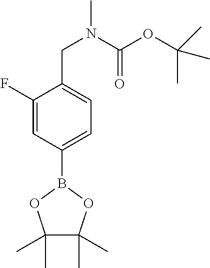 [SMILES: CN(Cc1ccc(B2OC(C)(C)C(C)(C)O2)cc1F)C(=O)OC(C)(C)C]

In a 250 mL round-bottomed flask, (4-Bromo-2-fluoro-benzyl)-methyl-carbamic acid tert-butyl ester (1.8 g, 5.66 mmol, Eq: 1.00), 4,4,4′,4′,5,5,5′,5′-octamethyl-2,2′-bi(1,3,2-dioxaborolane) (2.15 g, 8.49 mmol, Eq: 1.5) and potassium acetate (1.67 g, 17.0 mmol, Eq: 3.00) were combined with NMP (40 mL). The solution was degassed under nitrogen for 10 minutes. 1,1′-bis(diphenylphosphino) ferrocene]-dichloropalladium (II) (414 mg, 0.566 mmol, Eq: 0.1) was added and the reaction mixture heated at 100° C. for 24 hours. The reaction mixture was cooled down then diluted with water and EtOAc. The organic phases were combined then dried over anhydrous sodium sulfate. The solvent was removed under reduce pressure and the crude material obtained was purified by column chromatography (silica, 5-40% ethyl acetate in hexanes). The title compound was obtained as an oil (1.01 g, 49% yield).

### Step 3: 7-Benzenesulfonyl-4-chloro-6-(1-methyl-1H-pyrazol-4-yl)-7H-pyrrolo[2,3-d]pyrimidine

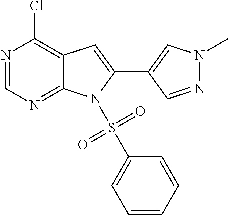 [SMILES: Cn1cc(-c2cc3c(Cl)ncnc3n2S(=O)(=O)c2ccccc2)cn1]

In a 20 mL scintillation vial, 4-chloro-6-iodo-7-(phenylsulfonyl)-7H-pyrrolo[2,3-d]pyrimidine (1 g, 2.38 mmol, Eq: 1.00), potassium carbonate (1.32 g, 9.53 mmol, Eq: 4.00) in 5 mL of water and 1-methyl-4-(4,4,5,5-tetramethyl-1,3,2-dioxaborolan-2-yl)-1H-pyrazole (744 mg, 3.57 mmol, Eq: 1.5) were combined with DME (10 mL). Pd(PPh₃)₄ (275 mg, 0.238 mmol, Eq: 0.1) was added and the reaction mixture was heated at 90° C. for 6 hours. The reaction mixture was let stand overnight at room temperature. A precipitate was formed which was filtered under vacuo to provide the title compound as a beige solid (194 mg, 22% yield). LC/MS: m/z calculated for C₁₆H₁₂ClN₅O₂S([M+H]⁺): 374.8 Found: 374.1

### Step 4: {2-Fluoro-4-[6-(1-methyl-1H-pyrazol-4-yl)-7H-pyrrolo[2,3-d]pyrimidin-4-yl]-benzyl}-methyl-carbamic acid tert-butyl ester

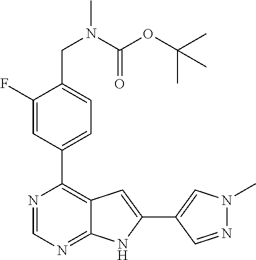 [SMILES: CN(Cc1ccc(-c2ncnc3[nH]c(-c4cnn(C)c4)cc23)cc1F)C(=O)OC(C)(C)C]

In a 20 mL scintillation vial, 7-benzenesulfonyl-4-chloro-6-(1-methyl-1H-pyrazol-4-yl)-7H-pyrrolo[2,3-d]pyrimidine (150 mg, 0.401 mmol, Eq: 1.00), [2-Fluoro-4-(4,4,5,5-tetramethyl-[1,3,2]dioxaborolan-2-yl)-benzyl]-methyl-carbamic acid tert-butyl ester (161 mg, 0.441 mmol, Eq: 1.1) and potassium carbonate (222 mg, 1.61 mmol, Eq: 4.00) in 3 mL of water were combined with DME (6 mL). Pd(PPh₃)₄ (46.4 mg, 0.04 mmol, Eq: 0.1) was added and the reaction mixture was heated at 160° C. for 60 minutes. The reaction mixture was diluted with EtOAc and washed with brine. The combined organic phases were dried over anhydrous sodium sulfate. The solvent was removed under reduced pressure and the crude material was purified by column chromatography (silica, 5-70% ethyl acetate in hexanes). The title compound was obtained as a solid (74 mg, 42% yield). LC/MS: m/z calculated for C₂₃H₂₅FN₆O₂([M+H]⁺): 437.4 Found: 437.3

### Step 5: 4-tert-Butyl-N-{2-fluoro-4-[6-(1-methyl-1H-pyrazol-4-yl)-7H-pyrrolo[2,3-d]pyrimidin-4-yl]-benzyl}-N-methyl-benzamide

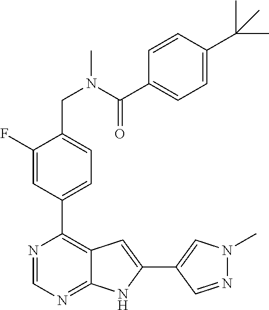 [SMILES: CN(Cc1ccc(-c2ncnc3[nH]c(-c4cnn(C)c4)cc23)cc1F)C(=O)c1ccc(C(C)(C)C)cc1]

In a 20 mL scintillation vial, {2-Fluoro-4-[6-(1-methyl-1H-pyrazol-4-yl)-7H-pyrrolo[2,3-d]pyrimidin-4-yl]-benzyl}-methyl-carbamic acid tert-butyl ester (40 mg, 0.092 mmol, Eq: 1.00) was combined with DCM (3 mL) and TFA (3 mL). The solution was stirred at room temperature for 30 minutes. The solvent was removed under reduced pressure. The crude material was dissolved in DMF (4 mL). 4-tert-Butyl-benzoic acid (18 mg, 0.101 mmol, Eq: 1.1), DIPEA (0.1 mL, 0.573 mmol, Eq: 6.25) and HATU (38 mg, 0.101 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The crude material purified by column chromatography (silica, 0-10% MeOH in DCM). The title compound was obtained as a solid (38 mg, 84% yield). LC/MS: m/z calculated for C₂₉H₂₉FN₆O([M+H]⁺): 497.5 Found: 497.4

### Example I-32

### 5-Methyl-thiophene-2-carboxylic acid {2-fluoro-4-[6-(1-methyl-1H-pyrazol-4-yl)-7H-pyrrolo[2,3-d]pyrimidin-4-yl]-benzyl}-methyl-amide

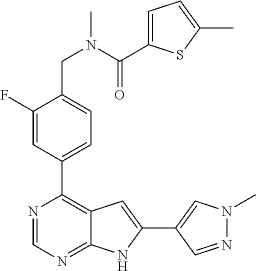 [SMILES: Cc1ccc(C(=O)N(C)Cc2ccc(-c3ncnc4[nH]c(-c5cnn(C)c5)cc34)cc2F)s1]

In a 20 mL scintillation vial, {2-Fluoro-4-[6-(1-methyl-1H-pyrazol-4-yl)-7H-pyrrolo[2,3-d]pyrimidin-4-yl]-benzyl}-methyl-carbamic acid tert-butyl ester (30 mg, 0.069 mmol, Eq: 1.00) was combined with DCM (3 mL) and TFA (3 mL). The solution was stirred at room temperature for 30 minutes. The solvent was removed under reduced pressure. The crude material was dissolved in DMF (4 mL). 5-methylthiophene-2-carboxylic acid (11 mg, 0.076 mmol, Eq: 1.1), DIPEA (0.05 mL, 0.275 mmol, Eq: 4.00) and HATU (29 mg, 0.076 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The crude material purified by column chromatography (silica, 0-10% MeOH in DCM). The title compound was obtained as a solid (14 mg, 44% yield). LC/MS: m/z calculated for C₂₄H₂₁FN₆OS ([M+H]⁺): 461.5 Found: 461.2

### Example I-33

### 2-tert-Butyl-5-{2-fluoro-4-[6-(1-methyl-1H-pyrazol-4-yl)-7H-pyrrolo[2,3-d]pyrimidin-4-yl]-benzyl}-4,5-dihydro-thieno[2,3-c]pyrrol-6-one

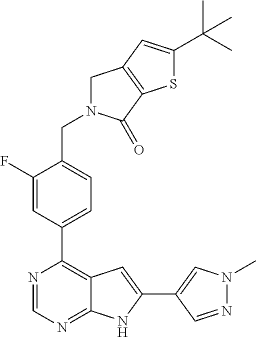 [SMILES: Cn1cc(-c2cc3c(-c4ccc(CN5Cc6cc(C(C)(C)C)sc6C5=O)c(F)c4)ncnc3[nH]2)cn1]

### Step 1: 3-Methyl-thiophene-2-carboxylic acid methyl ester

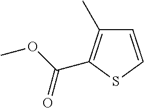

In a 1 L round-bottomed flask, 3-methylthiophene-2-carboxylic acid (15 g, 106 mmol) was combined with methanol (211 mL) to give an off-white suspension. This mixture was cooled to 0° C. in an ice-water bath. Concentrated sulfuric acid (6 ml, 113 mmol) was added dropwise to the cold suspension. The reaction mixture was stirred with gradual warming to room temperature. The reaction mixture was stirred at room temperature over three days. After this time, TLC showed complete conversion of the starting material to a less polar product. The reaction mixture was concentrated to remove methanol. The remaining light brown oil was partitioned between ethyl acetate and saturated aqueous sodium bicarbonate. The organic phase was dried over Na₂SO₄, filtered, and concentrated to afford a brown oil which contained a mixture of the desired methyl ester (84%) and the starting material (16%) based on ¹H NMR integration. The crude product was re-dissolved in ethyl acetate and the solution was washed with 1 M aqueous NaOH. The organic phase was dried over MgSO₄, filtered, and concentrated down to provide 3-methyl-thiophene-2-carboxylic acid methyl ester (13.6 g, 82%) as a light brown oil. ¹H NMR (300 MHz, CHLOROFORM-d) δ ppm 7.39 (d, J=5.09 Hz, 1H), 6.92 (d, J=5.20 Hz, 1H), 3.87 (s, 3H), 2.57 (s, 3H).

### Step 2: 5-tert-Butyl-3-methyl-thiophene-2-carboxylic acid methyl ester

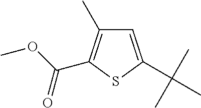

In a 500 mL round-bottom flask, aluminum trichloride (17.3 g, 130 mmol) was combined with DCM (20 mL) to give an off-white suspension. This mixture was back-filled with argon and then cooled to −78° C. in a dry ice/acetone bath. A solution of methyl 3-methylthiophene-2-carboxylate (13.5 g, 86.4 mmol) in 10 mL DCM was added dropwise over 5 minutes. The reaction mixture was stirred at −78° C. for 5 minutes. A solution of 2-chloro-2-methylpropane (9.87 mL, 90.7 mmol) in 10 mL DCM was added dropwise to the cold reaction mixture over 30 minutes. The reaction mixture was stirred over the weekend under a reflux condenser with the dry ice/acetone bath gradually melting and allowing the reaction flask to warm to room temperature. The reaction mixture was poured into ice water. After the ice melted, the organic phase was separated and then dried over Na₂SO₄. The organic phase was filtered then concentrated to afford a brown oil. This oil was loaded directly onto a 330 gram silica gel column. Flash chromatography (0-5% EtOAc-hexanes) was used to isolate 5-tert-butyl-3-methyl-thiophene-2-carboxylic acid methyl ester (7.05 g, 38%) as a yellow oil. ¹H NMR (300 MHz, CHLOROFORM-d) δ ppm 6.68 (s, 1H), 3.84 (s, 3H), 2.50 (s, 3H), 1.38 (s, 9H).

### Step 3: 3-Bromomethyl-5-tert-butyl-thiophene-2-carboxylic acid methyl ester

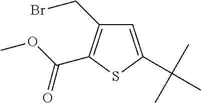

In a 1 L pear-shaped flask, methyl 5-tert-butyl-3-methylthiophene-2-carboxylate (6.06 g, 28.5 mmol), N-Bromosuccinimide (6.1 g, 34.3 mmol) and azobisisobutyronitrile (234 mg, 1.43 mmol) were combined with carbon tetrachloride (80 mL) to give an orange suspension. This mixture was heated at 90° C. overnight. In the morning, the reaction mixture was cooled to room temperature and then filtered to remove the precipitated solids. The filtrate was concentrated to a brown oil. This product was loaded directly onto a 120 gram silica gel column. Flash chromatography (5% EtOAc-hexanes) provided only partial purification of the product. The fractions containing pure product were consolidated to give 3-bromomethyl-5-tert-butyl-thiophene-2-carboxylic acid methyl ester (2.65 g, 32%) as a yellow oil. A second column was performed on the impure fractions from above, again using 120 g silica gel and 5% EtOAc-hexanes). Another lot of purified 3-bromomethyl-5-tert-butyl-thiophene-2-carboxylic acid methyl ester (2.54 g, 30%) was obtained. ¹H NMR (300 MHz, CHLOROFORM-d) δ ppm 6.93 (s, 1H), 4.87 (s, 2H), 3.88 (s, 3H), 1.39 (s, 8H).

### Step 4: 3-[(4-Bromo-2-fluoro-benzylamino)-methyl]-5-tert-butyl-thiophene-2-carboxylic acid methyl ester

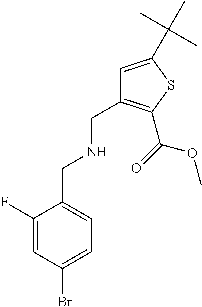 [SMILES: COC(=O)c1sc(C(C)(C)C)cc1CNCc1ccc(Br)cc1F]

In a 250 mL round-bottomed flask, 4-bromo-2-fluoro-benzylamine (5.34 g, 26.2 mmol), 3-bromomethyl-5-tert-butyl-thiophene-2-carboxylic acid methyl ester (2.54 g, 8.72 mmol) and cesium carbonate (3.73 g, 11.4 mmol) were combined with acetonitrile (50 mL) to give a white suspension. The reaction mixture was stirred over the weekend at room temperature. The reaction mixture was filtered, then the filtrate was concentrated on the rotary evaporator. The crude product was loaded directly onto a 120 gram silica gel column. Flash chromatography (5-25% EtOAc-hexanes) afforded 3-[(4-bromo-2-fluoro-benzylamino)-methyl]-5-tert-butyl-thiophene-2-carboxylic acid methyl ester (1.88 g, 52%) as a slightly yellow oil. LC/MS: m/z calculated for C₁₈H₂₂BrFN ([M+H]⁺): 414 and 416 Found: 416.0

### Step 5: 3-[(4-Bromo-2-fluoro-benzylamino)-methyl]-5-tert-butyl-thiophene-2-carboxylic acid

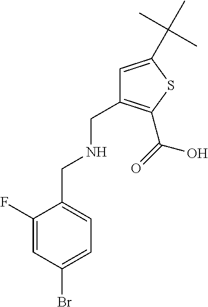 [SMILES: CC(C)(C)c1cc(CNCc2ccc(Br)cc2F)c(C(=O)O)s1]

In a 1 L pear-shaped flask, 3-[(4-bromo-2-fluoro-benzylamino)-methyl]-5-tert-butyl-thiophene-2-carboxylic acid methyl ester (1.85 g, 4.47 mmol) and lithium hydroxide monohydrate (1.87 g, 44.7 mmol) were combined with THF (12 mL) and water (12 mL) to give a colorless suspension.

This mixture was stirred at room temperature overnight. In the morning, LCMS shows mostly starting material and a small amount of product. Methanol (5 mL) was added and the reaction mixture was heated at 50° C. for 20 hours. The reaction mixture was cooled to room temperature and concentrated to dryness on the rotary evaporator. The resulting off-white solid was partially dissolved in water, then 4 N aqueous HCl was added until the mixture became a white suspension. This suspension was extracted with ethyl acetate. The organic phase was dried (Na₂SO₄), filtered, and then concentrated to afford 3-[(4-bromo-2-fluoro-benzylamino)-methyl]-5-tert-butyl-thiophene-2-carboxylic acid (1.77 g, 99%) as an off-white foam. LC/MS: m/z calculated for C₁₇H₂₀BrFNO ([M+H]⁺): 400 and 402 Found: 402.0

### Step 6: 5-(4-Bromo-2-fluoro-benzyl)-2-tert-butyl-4,5-dihydro-thieno[2,3-c]pyrrol-6-one

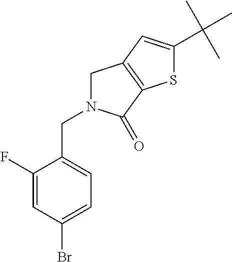 [SMILES: CC(C)(C)c1cc2c(s1)C(=O)N(Cc1ccc(Br)cc1F)C2]

In a 1 L round-bottomed flask, 3-((4-bromo-2-fluorobenzylamino)methyl)-5-tert-butylthiophene-2-carboxylic acid (1.77 g, 4.42 mmol) was combined with methylene chloride (80 mL) to give a light yellow solution. The reaction flask was back-filled with argon, then thionyl chloride (1.96 g, 1.2 mL, 16.4 mmol) was added dropwise over 5 minutes. The reaction mixture was stirred at room temperature under argon for 18 hours. After this time, LCMS shows a mixture of starting material and product. An additional 1.5 mL of thionyl chloride was added to the reaction mixture, and the reaction mixture was stirred for another 24 hours at room temperature. After this time, LCMS indicated that the reaction was completed. The reaction mixture was concentrated, giving a brownish-yellow oil. This crude product was dissolved in methylene chloride and the resulting solution was concentrated over silica gel. The silica gel supported crude product was loaded onto a 120 gram silica gel column. Flash chromatography (5-25% EtOAc-hexanes) afforded 5-(4-bromo-2-fluoro-benzyl)-2-tert-butyl-4,5-dihydro-thieno[2,3-c]pyrrol-6-one (1.22 g, 72%) as a slightly yellow oil. LC/MS: m/z calculated for C₁₇H₁₈BrFNOS ([M−H]⁺): 382 and 384 Found: 384.0

### Step 7: 2-tert-Butyl-5-[2-fluoro-4-(4,4,5,5-tetramethyl-[1,3,2]dioxaborolan-2-yl)-benzyl]-4,5-dihydro-thieno[2,3-c]pyrrol-6-one

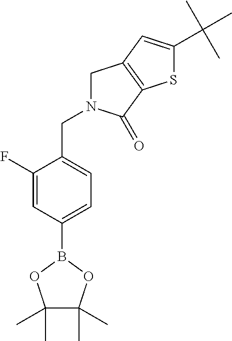 [SMILES: CC(C)(C)c1cc2c(s1)C(=O)N(Cc1ccc(B3OC(C)(C)C(C)(C)O3)cc1F)C2]

In a 250 mL round-bottomed flask, bis(pinacolato)diboron (1.15 g, 4.53 mmol), 5-(4-bromo-2-fluoro-benzyl)-2-tert-butyl-4,5-dihydro-thieno[2,3-c]pyrrol-6-one (1.07 g, 2.8 mmol) and potassium acetate (825 mg, 8.41 mmol) were combined with dioxane (9 mL) to give a dark brown suspension. To this mixture was added 1,1′-bis(diphenylphosphino)ferrocene-palladium(II)dichloride dichloromethane complex (185 mg, 227 μmol). The reaction mixture was heated at 110° C. for eight hours. After this time, the reaction mixture was cooled to room temperature and the dioxane was evaporated off. The crude product was dissolved in methylene chloride then the solution was poured into water (30 mL). The organic phase was separated and then dried over MgSO₄, filtered, and concentrated over silica gel. The silica gel supported crude product was loaded onto a 120 g silica gel column. Flash chromatography (5-25% ethyl acetate in hexanes) afforded 2-tert-butyl-5-[2-fluoro-4-(4,4,5,5-tetramethyl-[1,3,2]dioxaborolan-2-yl)-benzyl]-4,5-dihydro-thieno[2,3-c]pyrrol-6-one (0.72 g, 60%) as a white powder. LC/MS: m/z calculated for C₂₃H₃₀BFNO₃S ([M+H]⁺): 430 Found: 430.2

### Step 8: 2-tert-Butyl-5-{2-fluoro-4-[6-(1-methyl-1H-pyrazol-4-yl)-7H-pyrrolo[2,3-d]pyrimidin-4-yl]-benzyl}-4,5-dihydro-thieno[2,3-c]pyrrol-6-one

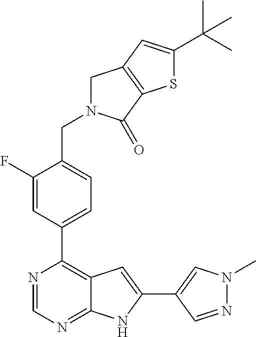 [SMILES: Cn1cc(-c2cc3c(-c4ccc(CN5Cc6cc(C(C)(C)C)sc6C5=O)c(F)c4)ncnc3[nH]2)cn1]

In a 10 mL microwave tube, 7-Benzenesulfonyl-4-chloro-6-(1-methyl-1H-pyrazol-4-yl)-7H-pyrrolo[2,3-d]pyrimidine (150 mg, 0.401 mmol, Eq: 1.00), 2-tert-Butyl-5-[2-fluoro-4-(4,4,5,5-tetramethyl-[1,3,2]dioxaborolan-2-yl)-benzyl]-4,5-dihydro-thieno[2,3-c]pyrrol-6-one (190 mg, 0.441 mmol, Eq: 1.1) and potassium carbonate (222 mg, 1.61 mmol, Eq: 4.00) in 2 mL of water were combined with DME (4 mL). Pd(Ph₃P)₄ (46 mg, 0.04 mmol, Eq: 0.1) was added. The reaction mixture was heated in a microwave at 160° C. for 60 minutes. The solution was diluted with EtOAc then washed with brine. The combined organic phases were dried over anhydrous sodium sulfate and the solvent was removed under reduced pressure. The crude material was purified by column chromatography (silica, 0-10% MeOH in DCM). The title compound was obtained as a light yellow solid (17 mg, 9% yield). LC/MS: m/z calculated for C₂₇H₂₅FN₆OS([M+H]⁺): 501.6 Found: 501.3

### Example I-34

### 5-tert-Butyl-isoxazole-3-carboxylic acid 2-fluoro-4-[6-(1-methyl-1H-pyrazol-4-yl)-7H-pyrrolo[2,3-d]pyrimidin-4-yl]-benzylamide

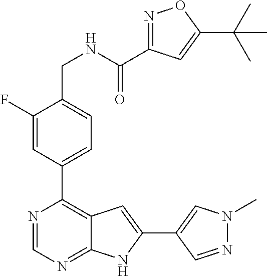 [SMILES: Cn1cc(-c2cc3c(-c4ccc(CNC(=O)c5cc(C(C)(C)C)on5)c(F)c4)ncnc3[nH]2)cn1]

In a 20 mL scintillation vial, {2-Fluoro-4-[6-(1-methyl-1H-pyrazol-4-yl)-7H-pyrrolo[2,3-d]pyrimidin-4-yl]-benzyl}-methyl-carbamic acid tert-butyl ester (75 mg, 0.178 mmol, Eq: 1.00) was combined with DCM (3 mL) and TFA (3 mL). The solution was stirred at room temperature for 30 minutes. The solvent was removed under reduced pressure. The crude material was dissolved in DMF (4 mL). 5-tert-butylisoxazole-3-carboxylic acid (18 mg, 0.195 mmol, Eq: 1.1), DIPEA (0.124 mL, 0.710 mmol, Eq: 4.00) and HATU (74 mg, 0.195 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were combined and dried over anhydrous sodium sulfate. The solvent was removed under reduced pressure. The crude material was purified by column chromatography (silica, 0-10% MeOH in DCM). The title compound was obtained as a yellow solid (35 mg, 42% yield). LC/MS: m/z calculated for C₂₅H₂₄FN₇O₂([M+H]⁺): 474.5 Found: 474.3

### Example I-35

### N-{2-Fluoro-4-[6-(1-methyl-1H-pyrazol-4-yl)-7H-pyrrolo[2,3-d]pyrimidin-4-yl]-benzyl}-4-(3-methyl-oxetan-3-yl)-benzamide

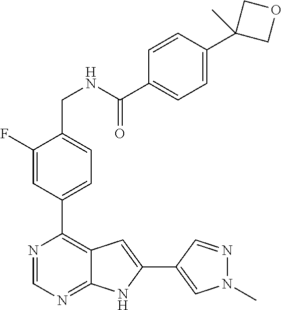 [SMILES: Cn1cc(-c2cc3c(-c4ccc(CNC(=O)c5ccc(C6(C)COC6)cc5)c(F)c4)ncnc3[nH]2)cn1]

In a 10 mL sealable tube, {2-Fluoro-4-[6-(1-methyl-1H-pyrazol-4-yl)-7H-pyrrolo[2,3-d]pyrimidin-4-yl]-benzyl}-methyl-carbamic acid tert-butyl ester (65 mg, 0.155 mmol, Eq: 1.00) was combined with DCM (3 mL) and TFA (3 mL). The solution was stirred at room temperature for 60 minutes. The solvent was removed under reduced pressure and dried under vacuo. The crude material was dissolved in DMF (5 mL). 4-(3-methyloxetan-3-yl)benzoic acid (59.6 mg, 0.31 mmol, Eq: 2.00), DIPEA (0.14 mL, 0.78 mmol, Eq: 5.00) and HATU (118 mg, 0.31 mmol, Eq: 2.00) were added. The reaction mixture was stirred at room temperature overnight. The resulting solution was diluted with EtOAc, washed with water and brine. The combined organic phases were dried over anhydrous sodium sulfate. The solvent was removed under reduced pressure. The crude material was purified by column chromatography (silica, 0-10% MeOH in DCM). The title compound was obtained as a solid (44 mg, 57% yield). LC/MS: m/z Calculated for C₂₈H₂₅FN₆O₂ ([M+H]⁺): 497.5 Found: 497.2

### Example I-36

### 4-(Cyano-dimethyl-methyl)-N-{2-fluoro-4-[6-(1-methyl-1H-pyrazol-4-yl)-7H-pyrrolo[2,3-d]pyrimidin-4-yl]-benzyl}-benzamide

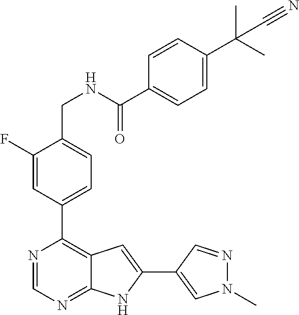 [SMILES: Cn1cc(-c2cc3c(-c4ccc(CNC(=O)c5ccc(C(C)(C)C#N)cc5)c(F)c4)ncnc3[nH]2)cn1]

Following a similar procedure described in example 35 using 4-(2-cyanopropan-2-yl)benzoic acid (58.7 mg, 0.310 mmol, Eq: 2.00), the title compound was obtained as a solid (43 mg, 53% yield). LC/MS: m/z Calculated for C₂₈H₂₄FN₇O ([M+H]⁺): 494.5 Found: 494.2

### Example I-37

### 4,5,6,7-Tetrahydro-benzo[b]thiophene-2-carboxylic acid 2-fluoro-4-[6-(1-methyl-1H-pyrazol-4-yl)-7H-pyrrolo[2,3-d]pyrimidin-4-yl]-benzylamide

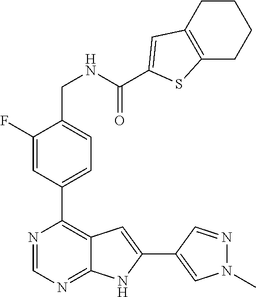 [SMILES: Cn1cc(-c2cc3c(-c4ccc(CNC(=O)c5cc6c(s5)CCCC6)c(F)c4)ncnc3[nH]2)cn1]

Following a similar procedure described in example 35 using 4,5,6,7-tetrahydrobenzo[b]thiophene-2-carboxylic acid (57 mg, 0.31 mmol, Eq: 2.00), the title compound was obtained as a solid (53 mg, 63% yield). LC/MS: m/z Calculated for C₂₆H₂₃FN₆OS ([M+H]⁺): 487.5 Found: 487.2

### Example I-38

### N-{2-Fluoro-4-[6-(1-methyl-1H-pyrazol-4-yl)-7H-pyrrolo[2,3-d]pyrimidin-4-yl]-benzyl}-4-(1-hydroxy-1-methyl-ethyl)-benzamide

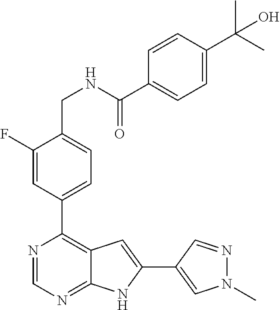 [SMILES: Cn1cc(-c2cc3c(-c4ccc(CNC(=O)c5ccc(C(C)(C)O)cc5)c(F)c4)ncnc3[nH]2)cn1]

### Step 1: 4-(1-Hydroxy-1-methyl-ethyl)-benzoic acid

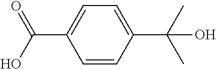

In a 500 mL round-bottomed flask, 4-isopropylbenzoic acid (1.0 g, 6.09 mmol) was combined with 5 mL of 10% KOH in water to give a cloudy suspension. KOH in water (96 mL, 19.2 mmol) and potassium permanganate (1.92 g, 12.2 mmol) in 100 mL water were added. The reaction mixture was heated at 70° C. for 1 hour. To the reaction mixture was added 5 drops of glycerol. The reaction mixture was cooled to 0° C. The solid residue was filtered through a celite pad. The filtrate was washed twice with ether. The combined organic phases were rinsed with brine, dried over anhydrous sodium sulfate, concentrated and dried overnight. The product 4-(1-hydroxy-1-methyl-ethyl)-benzoic acid was collected as a white solid (870 mg, 79%) which used in the next step without further purification. ¹H NMR (400 MHz, DMSO-d₆) δ ppm 12.80 (br. s, 1H), 7.83-7.89 (m, 2H), 7.55-7.60 (m, 2H), 5.15 (s, 1H), 1.43 (s, 6H).

### Step 2: N-{2-Fluoro-4-[6-(1-methyl-1H-pyrazol-4-yl)-7H-pyrrolo[2,3-d]pyrimidin-4-yl]-benzyl}-4-(1-hydroxy-1-methyl-ethyl)-benzamide

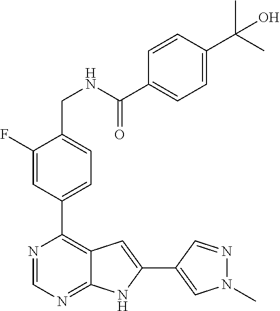 [SMILES: Cn1cc(-c2cc3c(-c4ccc(CNC(=O)c5ccc(C(C)(C)O)cc5)c(F)c4)ncnc3[nH]2)cn1]

Following a similar procedure described in example 35 using 4-(2-hydroxypropan-2-yl)benzoic acid (41.4 mg, 0.230 mmol, Eq: 2.00), the title compound was obtained as a solid (46 mg, 82% yield). LC/MS: m/z Calculated for C₂₇H₂₅FN₆O₂ ([M+H]⁺): 485.5 Found: 485.4

### Example I-39

### 3-tert-Butyl-isoxazole-5-carboxylic acid 2-fluoro-4-[6-(1-methyl-1H-pyrazol-4-yl)-7H-pyrrolo[2,3-d]pyrimidin-4-yl]-benzyl amide

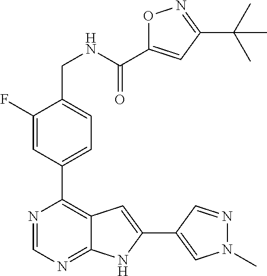 [SMILES: Cn1cc(-c2cc3c(-c4ccc(CNC(=O)c5cc(C(C)(C)C)no5)c(F)c4)ncnc3[nH]2)cn1]

### Step 1: 3-tert-Butyl-isoxazole-5-carboxylic acid methyl ester

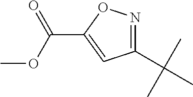

To a solution of pivaldehyde (1.0 g, 11.6 mmol) in 1:1 t-butanol/water (40 mL) was added hydroxylamine hydrochloride (807 mg, 11.6 mmol) and sodium hydroxide (464 mg, 11.6 mmol). The mixture was stirred at room temperature for 30 minutes before chloramine-T (5.49 g, 23.4 mmol) was added in portions over 5 minutes followed by copper(II) sulfate (327 mg, 1.3 mmol) and copper powder (73.8 mg, 1.16 mmol) and methyl propiolate (976 mg, 11.6 mmol). The reaction mixture is heated at reflux where it is maintained for 2 h. After this time, the mixture is cooled to room temperature and poured onto ice/water (50 g). Ammonium hydroxide (10 mL) was added and the solution was extracted with DCM (3×200 mL). The organic layers are combined, dried (Na₂SO₄), filtered and concentrated under reduced pressure. The crude material was purified by flash chromatography (silica gel, 40 g, 0% to 10% EtOAc in hexanes) to give 3-tert-butyl-isoxazole-5-carboxylic acid methyl ester (427 mg, 20%) as a colorless oil. LC/MS: m/z Calculated for C₉H₁₃NO₃ [(M+H)⁺] 184 Found: 184.1

### Step 2: 3-tert-Butyl-isoxazole-5-carboxylic acid

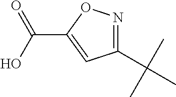

To a solution of 3-tert-butyl-isoxazole-5-carboxylic acid methyl ester (425 mg, 2.32 mmol) in methanol (4 mL) was added 1 N aqueous NaOH (11.6 ml, 11.6 mmol). The reaction mixture was stirred at room temperature for 2 hour, then concentrated and neutralized with 1 N hydrochloric acid (10 mL). The mixture was extracted with ethyl acetate, dried over sodium sulfate, filtered, and concentrated to give 3-tert-butyl-isoxazole-5-carboxylic acid (318 mg, 81%) as a white semi-solid. LC/MS: m/z Calculated for C₈H₁₁NO₃ [(M+H)⁺] 170 Found: 170

### Step 3: 3-tert-Butyl-isoxazole-5-carboxylic acid 2-fluoro-4-[6-(1-methyl-1H-pyrazol-4-yl)-7H-pyrrolo[2,3-d]pyrimidin-4-yl]-benzyl amide

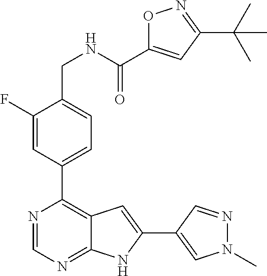 [SMILES: Cn1cc(-c2cc3c(-c4ccc(CNC(=O)c5cc(C(C)(C)C)no5)c(F)c4)ncnc3[nH]2)cn1]

Following a similar procedure described in example 35 using 3-tert-butylisoxazole-5-carboxylic acid (39 mg, 0.230 mmol, Eq: 2.00), the title compound was obtained as a solid (39 mg, 71% yield). LC/MS: m/z Calculated for C₂₅H₂₄FN₇O₂ ([M+H]⁺): 474.5. Found: 474.3.

### Example I-40

### 3-tert-Butoxy-azetidine-1-carboxylic acid 2-fluoro-4-[6-(1-methyl-1H-pyrazol-4-yl)-7H-pyrrolo[2,3-d]pyrimidin-4-yl]-benzylamide

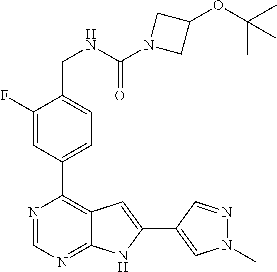 [SMILES: Cn1cc(-c2cc3c(-c4ccc(CNC(=O)N5CC(OC(C)(C)C)C5)c(F)c4)ncnc3[nH]2)cn1]

In a 20 ml sealable microwave tube, {2-fluoro-4-[6-(1-methyl-1H-pyrazol-4-yl)-7H-pyrrolo[2,3-d]pyrimidin-4-yl]-benzyl}-methyl-carbamic acid tert-butyl ester (65 mg, 0.155 mmol, Eq: 1.00) was combined with DCM (3 mL) and TFA (3 mL). The solution was stirred at room temperature for 60 minutes. The solvent was removed under reduced pressure and dried under vacuo. The crude material was dissolved in DMF (4 mL). Di(1H-imidazol-1-yl)methanone (50.3 mg, 0.31 mmol, Eq: 2.00) and DIPEA (0.14 mL, 0.78 mmol, Eq: 5.00) were added to the reaction mixture to give a light yellow solution. The reaction was stirred at room temperature for 2 hrs. 3-tert-butoxyazetidine (40 mg, 0.310 mmol, Eq: 2.00) was added and stirred at room temperature overnight. The reaction mixture was diluted with EtOAc and washed with brine. The organic phases were combined and dried over anhydrous sodium sulfate. The solvent was removed under reduced pressure. The crude material was purified by column chromatography (silica, 0-10% MeOH in DCM). The title compound was obtained as a yellow solid (32 mg, 43% yield). LC/MS: m/z Calculated for C₂₅H₂₈FN₇O₂ ([M−H]⁺): 478.5 Found: 478.6

### Example I-41

### 1,3-Dihydro-isoindole-2-carboxylic acid 2-fluoro-4-[6-(1-methyl-1H-pyrazol-4-yl)-7H-pyrrolo[2,3-d]pyrimidin-4-yl]-benzylamide

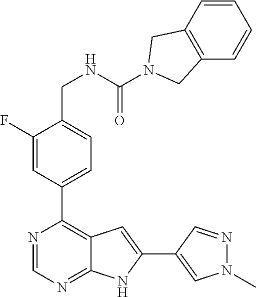 [SMILES: Cn1cc(-c2cc3c(-c4ccc(CNC(=O)N5Cc6ccccc6C5)c(F)c4)ncnc3[nH]2)cn1]

Following a similar procedure described in example 40 using isoindoline (37.0 mg, 0.31 mmol, Eq: 2.00), the title compound was obtained as a solid (43 mg, 59% yield). LC/MS: m/z Calculated for C₂₆H₂₂FN₇O ([M+H]⁺): 468.5 Found: 468.1

### Example 42

### 4-tert-Butyl-N-(4-{6-[1-(2-dimethylamino-ethyl)-1H-pyrazol-4-yl]-7H-pyrrolo[2,3-d]pyrimidin-4-yl}-2-fluoro-benzyl)-benzamide

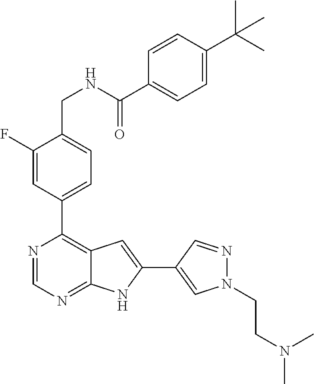 [SMILES: CN(C)CCn1cc(-c2cc3c(-c4ccc(CNC(=O)c5ccc(C(C)(C)C)cc5)c(F)c4)ncnc3[nH]2)cn1]

### Step 1: N,N-Dimethyl-2-(4-(4,4,5,5-tetramethyl-1,3,2-dioxaborolan-2-yl)-1H-pyrazol-1-yl)-ethanamine

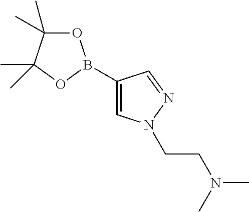

In a 250 mL round-bottomed flask, 2-chloro-N,N-dimethylethanamine (998 mg, 9.28 mmol), 4-(4,4,5,5-tetramethyl-1,3,2-dioxaborolan-2-yl)-1H-pyrazole (1.2 g, 6.18 mmol) and cesium carbonate (4.03 g, 12.4 mmol) were combined with acetonitrile (20 mL) to give a white suspension. The reaction mixture was heated at 100° C. overnight. In the morning, the reaction mixture was cooled to room temperature, filtered, and the filtrate was concentrated to give title compound (1.26 g, 77% yield) as a colorless oil. The crude product was used in subsequent reactions without purification.

### Step 2: (4-{6-[1-(2-Dimethylamino-ethyl)-1H-pyrazol-4-yl]-7H-pyrrolo[2,3-d]pyrimidin-4-yl}-2-fluorobenzyl)-carbamic acid tert-butyl ester

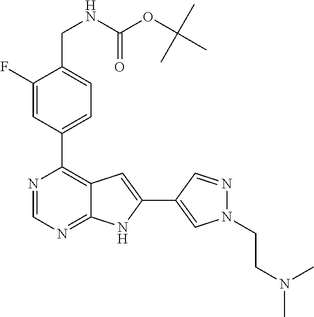 [SMILES: CN(C)CCn1cc(-c2cc3c(-c4ccc(CNC(=O)OC(C)(C)C)c(F)c4)ncnc3[nH]2)cn1]

In a 20 mL sealable microwave tube, tert-butyl 4-(6-bromo-7-tosyl-7H-pyrrolo[2,3-d]pyrimidin-4-yl)-2-fluorobenzylcarbamate (250 mg, 0.434 mmol, Eq: 1.00), N,N-dimethyl-2-(4-(4,4,5,5-tetramethyl-1,3,2-dioxaborolan-2-yl)-1H-pyrazol-1-yl)ethanamine (319 mg, 1.2 mmol, Eq: 2.77) and Pd(PPh₃)₄ (50 mg, 0.043 mmol, Eq: 0.1) were combined with DME (4 ml) to give a light brown suspension. Water (1 mL) was added, followed by potassium carbonate (240 mg, 1.74 mmol, Eq: 4.00). The reaction mixture was heated at 150° C. microwave for 1 hr. The reaction mixture was diluted with EtOAc and washed with brine and water. The organic phases were combined and dried over anhydrous sodium sulfate. The solvent was removed under reduced pressure. The crude material was purified by column chromatography (silica, 0-10% MeOH in DCM). The title compound was obtained as a yellow solid (62 mg, 29% yield). LC/MS: m/z Calculated for C₂₅H₃₀FN₇O₂ ([M+H]⁺): 480.6 Found: 480.3

### Step 3: (2-{4-[4-(4-Aminomethyl-3-fluoro-phenyl)-7H-pyrrolo[2,3-d]pyrimidin-6-yl]-pyrazol-1-yl}-ethyl)-dimethyl-amine

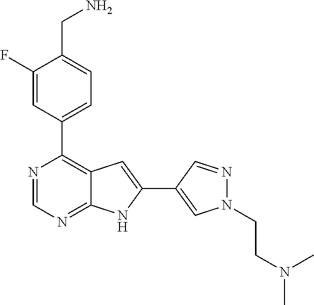 [SMILES: CN(C)CCn1cc(-c2cc3c(-c4ccc(CN)c(F)c4)ncnc3[nH]2)cn1]

In a 100 mL round-bottomed flask, tert-butyl 4-(6-(1-(2-(dimethylamino)ethyl)-1H-pyrazol-4-yl)-7H-pyrrolo[2,3-d]pyrimidin-4-yl)-2-fluorobenzylcarbamate (62 mg, 0.129 mmol, Eq: 1.00) was combined with DCM (8 mL) to give a light yellow suspension. TFA (4 mL, 51.9 mmol, Eq: 402) was added and stirred at room temperature for 1 hr. The solvent was removed under reduced pressure. The crude material was dried on high vacuum for 2 hrs. The residue was used in next step without further purification. LC/MS: m/z Calculated for C₂₀H₂₂FN₇ ([M+H]⁺): 380.4 Found: 380.2

### Step 4: 4-tert-Butyl-N-(4-{6-[1-(2-dimethylamino-ethyl)-1H-pyrazol-4-yl]-7H-pyrrolo[2,3-d]pyrimidin-4-yl}-2-fluoro-benzyl)-benzamide

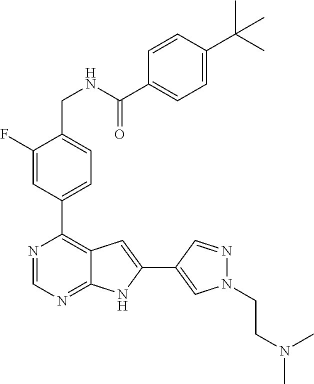 [SMILES: CN(C)CCn1cc(-c2cc3c(-c4ccc(CNC(=O)c5ccc(C(C)(C)C)cc5)c(F)c4)ncnc3[nH]2)cn1]

In a 10 mL sealable tube, (2-{4-[4-(4-Aminomethyl-3-fluoro-phenyl)-7H-pyrrolo[2,3-d]pyrimidin-6-yl]-pyrazol-1-yl}-ethyl)-dimethyl-amine (48 mg, 0.127 mmol, Eq: 1.00), 4-tert-butylbenzoic acid (45 mg, 0.253 mmol, Eq: 2.00) and HATU (96 mg, 0.253 mmol, Eq: 2.00) were combined with DMF (4 mL) to give a yellow solution. The reaction mixture was stirred for 5 minutes then DIPEA (0.110 ml, 0.630 mmol, Eq: 5.00) was added and stirred at room temperature overnight. The reaction mixture was diluted with EtOAc and washed with brine. The organic phases were combined and dried over anhydrous sodium sulfate. The solvent was removed under reduced pressure. The crude material was purified by column chromatography (silica, 0-10% MeOH in DCM). The title compound was obtained as a solid (49 mg, 71% yield). LC/MS: m/z Calculated for C₃₁H₃₄FN₇O ([M+H]⁺): 540.6 Found: 540.3

### Example I-43

### 3-tert-Butoxy-azetidine-1-carboxylic acid 4-{6-[1-(2-dimethylamino-ethyl)-1H-pyrazol-4-yl]-7H-pyrrolo[2,3-d]pyrimidin-4-yl}-2-fluoro-benzylamide

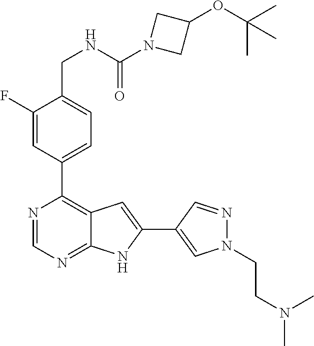 [SMILES: CN(C)CCn1cc(-c2cc3c(-c4ccc(CNC(=O)N5CC(OC(C)(C)C)C5)c(F)c4)ncnc3[nH]2)cn1]

In a 20 mL sealable microwave tube, (2-{4-[4-(4-Aminomethyl-3-fluoro-phenyl)-7H-pyrrolo[2,3-d]pyrimidin-6-yl]-pyrazol-1-yl}-ethyl)-dimethyl-amine (22 mg, 0.056 mmol, Eq: 1.00), di(1H-imidazol-1-yl)methanone (19 mg, 0.116 mmol, Eq: 2.00) and DIPEA (51 μl, 0.290 mmol, Eq: 5.00) were combined with DMF (2 mL) to give a light yellow solution. The reaction was stirred at room temperature for 2 hrs. 3-tert-butoxyazetidine (15.0 mg, 0.116 mmol, Eq: 2.00) was added and stirred at room temperature overnight. The reaction mixture was diluted with EtOAc and washed with brine and water. The organic phases were combined and dried over anhydrous sodium sulfate. The solvent was removed under reduced pressure. The crude material was purified by column chromatography (silica, 0-10% MeOH in DCM). The title compound was obtained as a yellow solid (18 mg, 58% yield). LC/MS: m/z Calculated for C₂₈H₃₅FN₈O₂ ([M−H]⁺): 535.6. Found: 535.4.

### Example I-44

### 1,3-Dihydro-isoindole-2-carboxylic acid 4-{6-[1-(2-dimethylamino-ethyl)-1H-pyrazol-4-yl]-7H-pyrrolo[2,3-d]pyrimidin-4-yl}-2-fluoro-benzylamide

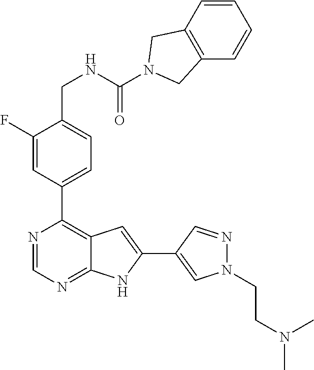 [SMILES: CN(C)CCn1cc(-c2cc3c(-c4ccc(CNC(=O)N5Cc6ccccc6C5)c(F)c4)ncnc3[nH]2)cn1]

Following a similar procedure described in example 43 using isoindoline (14 mg, 0.116 mmol, Eq: 2.00), the title compound was obtained as a yellow solid (20 mg, 63% yield). LC/MS: m/z Calculated for C₂₉H₂₉FN₈O ([M+H]⁺): 525.6 Found: 525.3

### Example I-45

### [4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid ethyl ester

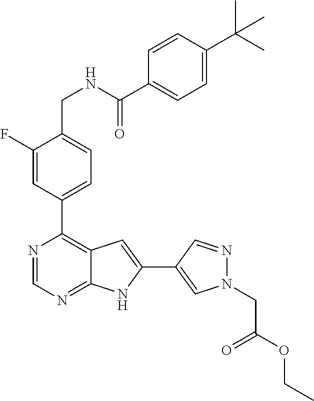 [SMILES: CCOC(=O)Cn1cc(-c2cc3c(-c4ccc(CNC(=O)c5ccc(C(C)(C)C)cc5)c(F)c4)ncnc3[nH]2)cn1]

### Step 1: (4-{4-[4-(tert-Butoxycarbonylamino-methyl)-3-fluoro-phenyl]-7H-pyrrolo[2,3-d]pyrimidin-6-yl}-pyrazol-1-yl)-acetic acid ethyl ester

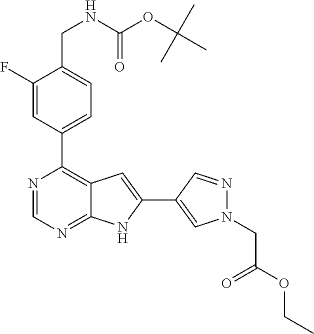 [SMILES: CCOC(=O)Cn1cc(-c2cc3c(-c4ccc(CNC(=O)OC(C)(C)C)c(F)c4)ncnc3[nH]2)cn1]

In a 20 mL sealable microwave tube, tert-butyl 4-(6-bromo-7-tosyl-7H-pyrrolo[2,3-d]pyrimidin-4-yl)-2-fluorobenzylcarbamate (250 mg, 0.434 mmol, Eq: 1.00), ethyl 2-(4-(4,4,5,5-tetramethyl-1,3,2-dioxaborolan-2-yl)-1H-pyrazol-1-yl)acetate (365 mg, 1.3 mmol, Eq: 3) and Pd(PPh₃)₄ (50 mg, 0.043 mmol, Eq: 0.1) were combined with DMF (10 mL) to give a light brown suspension. Potassium carbonate (240 mg, 1.74 mmol, Eq: 4.00) was added. The reaction mixture was heated at 155° C. microwave for 1 hr. The reaction mixture was diluted with EtOAc and washed with brine and water. The organic phases were combined and dried over anhydrous sodium sulfate. The solvent was removed under reduced pressure. The crude material was purified by column chromatography (silica, 5-60% EtOAc in hexanes). The title compound was obtained as a solid (72 mg, 33% yield). LC/MS: m/z Calculated for C₂₅H₂₇FN₆O₄ ([M+H]⁺): 495.5 Found: 495.3

### Step 2: {4-[4-(4-Aminomethyl-3-fluoro-phenyl)-7H-pyrrolo[2,3-d]pyrimidin-6-yl]-pyrazol-1-yl}-acetic acid ethyl ester

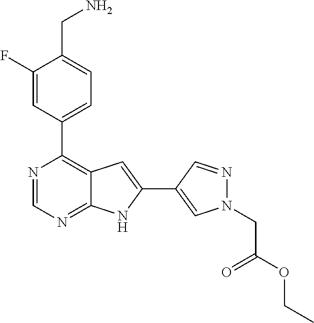 [SMILES: CCOC(=O)Cn1cc(-c2cc3c(-c4ccc(CN)c(F)c4)ncnc3[nH]2)cn1]

In a 50 mL round-bottomed flask, (4-{4-[4-(tert-Butoxycarbonylamino-methyl)-3-fluoro-phenyl]-7H-pyrrolo[2,3-d]pyrimidin-6-yl}-pyrazol-1-yl)-acetic acid ethyl ester (72 mg, 0.146 mmol, Eq: 1.00) was combined with DCM (8 mL) to give a yellow solution. TFA (4 mL, 51.9 mmol, Eq: 357) was added and stirred at room temperature for 1 hr. The solvent was removed under reduced pressure, further dried on vacuum for 3 hrs. The residue was used in next step without further purification. LC/MS: m/z Calculated for C₂₀H₁₉FN₆O₂ ([M−H]⁺): 395.4. Found: 395.2.

### Step 3: [4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid ethyl ester

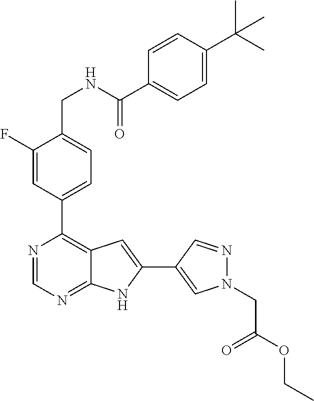 [SMILES: CCOC(=O)Cn1cc(-c2cc3c(-c4ccc(CNC(=O)c5ccc(C(C)(C)C)cc5)c(F)c4)ncnc3[nH]2)cn1]

In a 50 mL round bottom flask, {4-[4-(4-Aminomethyl-3-fluoro-phenyl)-7H-pyrrolo[2,3-d]pyrimidin-6-yl]-pyrazol-1-yl}-acetic acid ethyl ester (52 mg, 0.132 mmol, Eq: 1.00), 4-tert-butylbenzoic acid (47.0 mg, 0.264 mmol, Eq: 2.00) and HATU (100 mg, 0.264 mmol, Eq: 2.00) were combined with DMF (4 mL) to give a yellow solution. The reaction mixture was stirred for 5 minutes then DIPEA (115 μl, 659 μmol, Eq: 5.00) was added and stirred at room temperature overnight. The reaction mixture was diluted with EtOAc and washed with brine and water. The organic phases were combined and dried over anhydrous sodium sulfate. The solvent was removed under reduced pressure. The crude material was purified by column chromatography (silica, 0-10% MeOH in DCM). The title compound was obtained as a solid (52 mg, 69% yield). LC/MS: m/z Calculated for C₃₁H₃₁FN₆O₃ ([M+H]⁺): 555.6 Found: 555.4

### Example 46

### [4-(4-{4-[(4-tert-Butyl-benzoylamino)-methyl]-3-fluoro-phenyl}-7H-pyrrolo[2,3-d]pyrimidin-6-

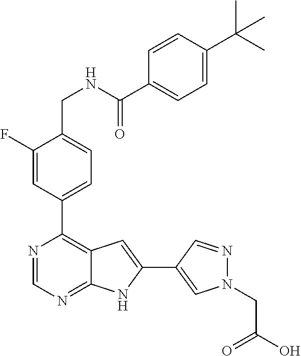 [SMILES: CC(C)(C)c1ccc(C(=O)NCc2ccc(-c3ncnc4[nH]c(-c5cnn(CC(=O)O)c5)cc34)cc2F)cc1]

In a 50 mL round bottom flask, [4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid ethyl ester (47 mg, 0.085 mmol, Eq: 1.00) were combined with THF (5 mL) to give a yellow suspension. NaOH 1M solution (0.135 ml, 0.135 mmol, Eq: 1.59) was added and stirred at room temperature overnight. The reaction was acidified by addition of 1N HCl. The solvent was removed under reduced pressure. The residue was purified by HPLC to provide the title compound as a yellow solid (34 mg, 76% yield). LC/MS: m/z Calculated for C₂₉H₂₇FN₆O₃ ([M+H]⁺): 527.6 Found: 527.3

### Example 47

### N-(2-fluoro-4-(6-(1-methyl-1H-pyrazol-4-yl)-7H-pyrrolo[2,3-d]pyrimidin-4-yl)benzyl)-4,5,6,7-tetrahydropyrazolo[1,5-a]pyridine-2-carboxamide

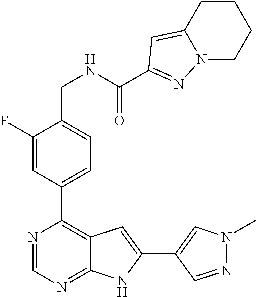 [SMILES: Cn1cc(-c2cc3c(-c4ccc(CNC(=O)c5cc6n(n5)CCCC6)c(F)c4)ncnc3[nH]2)cn1]

### Step 1: N-(4-bromo-2-fluorobenzyl)-4,5,6,7-tetrahydropyrazolo[1,5-a]pyridine-2-carboxamide

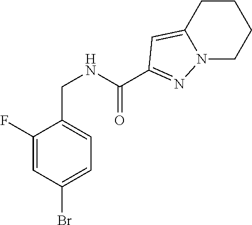 [SMILES: O=C(NCc1ccc(Br)cc1F)c1cc2n(n1)CCCC2]

(4-bromo-2-fluorophenyl)methanamine (193 mg, 945 μmol, Eq: 1.00), 4,5,6,7-tetrahydropyrazolo[1,5-a]pyridine-2-carboxylic acid (157 mg, 945 μmol, Eq: 1.00), HBTU (358 mg, 945 μmol, Eq: 1.00) and DIPEA (366 mg, 495 μl, 2.83 mmol, Eq: 3) in DMF (3.15 ml) was stirred at r.t. for 16 h. The reaction mixture was diluted with ethyl acetate, washed with water and brine. The combined organic layers were dried over anhydrous sodium sulfate. The solvent was removed under reduced pressure. The crude material obtained was purified by column chromatography (silica, 10-65% ethyl acetate in hexanes) to give N-(4-bromo-2-fluorobenzyl)-4,5,6,7-tetrahydropyrazolo[1,5-a]pyridine-2-carboxamide (178 mg, 54% yield) as a colorless oil. LC/MS: m/z calculated for C₁₅H₁₅BrFN₃O([M+H]⁺): 353.2 Found: 354.1

### Step 2: N-(2-fluoro-4-(4,4,5,5-tetramethyl-1,3,2-dioxaborolan-2-yl)benzyl)-4,5,6,7-tetrahydropyrazolo[1,5-a]pyridine-2-carboxamide

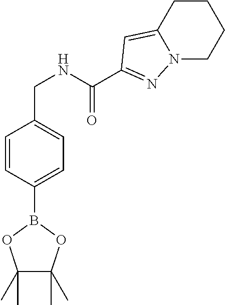 [SMILES: CC1(C)OB(c2ccc(CNC(=O)c3cc4n(n3)CCCC4)cc2)OC1(C)C]

N-(4-bromo-2-fluorobenzyl)-4,5,6,7-tetrahydropyrazolo[1,5-a]pyridine-2-carboxamide (178 mg, 505 μmol, Eq: 1.00), 4,4,4′,4′,5,5,5′,5′-octamethyl-2,2′-bi(1,3,2-dioxaborolane) (193 mg, 758 μmol, Eq: 1.5), PdCl₂(dppf)-CH₂Cl₂ adduct (37.0 mg, 50.5 μmol, Eq: 0.1) and potassium acetate (149 mg, 1.52 mmol, Eq: 3) in NMP (3 mL) was heated to 100° C. for 16 h. The reaction mixture was diluted with ethyl acetate, washed with water and brine. The combined organic layers were dried over anhydrous sodium sulfate and the solvent was removed under reduced pressure. The crude material obtained was purified by column chromatography (silica, 30-100% ethyl acetate in hexanes) to give N-(2-fluoro-4-(4,4,5,5-tetramethyl-1,3,2-dioxaborolan-2-yl)benzyl)-4,5,6,7-tetrahydropyrazolo[1,5-a]pyridine-2-carboxamide (74 mg, 37% yield) as a white solid. LC/MS: m/z calculated for C₂₁H₂₇BFN₃O₃([M+H]⁺): 400.2 Found: 400.2

### Step 3: N-(2-fluoro-4-(6-(1-methyl-1H-pyrazol-4-yl)-7H-pyrrolo[2,3-d]pyrimidin-4-yl)benzyl)-4,5,6,7-tetrahydropyrazolo[1,5-a]pyridine-2-carboxamide

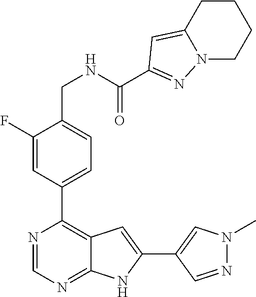 [SMILES: Cn1cc(-c2cc3c(-c4ccc(CNC(=O)c5cc6n(n5)CCCC6)c(F)c4)ncnc3[nH]2)cn1]

4-chloro-6-(1-methyl-1H-pyrazol-4-yl)-7-(phenylsulfonyl)-7H-pyrrolo[2,3-d]pyrimidine (69.3 mg, 185 μmol, Eq: 1.00), N-(2-fluoro-4-(4,4,5,5-tetramethyl-1,3,2-dioxaborolan-2-yl)benzyl)-4,5,6,7-tetrahydropyrazolo[1,5-a]pyridine-2-carboxamide (74 mg, 185 μmol, Eq: 1.00), tetrakis(triphenylphosphine)palladium (0) (21.4 mg, 18.5 μmol, Eq: 0.1) and potassium carbonate (76.8 mg, 556 μmol, Eq: 3) in DME (1.48 ml)/Water (371 μl) was heated to 150° C. in the microwave for 45 min. Purified by column chromatography (silica, 0-100% ethyl acetate in [10% MeOH/ethyl acetate]) followed by HPLC purification to give N-(2-fluoro-4-(6-(1-methyl-1H-pyrazol-4-yl)-7H-pyrrolo[2,3-d]pyrimidin-4-yl)benzyl)-4,5,6,7-tetrahydropyrazolo[1,5-a]pyridine-2-carboxamide (5.7 mg, 7% yield) as a yellow solid. ¹H NMR (400 MHz, DMSO-d) 6 ppm 12.63 (s, 1H), 8.79 (s, 1H), 8.73 (t, J=5.8 Hz, 1H), 8.31 (s, 1H), 8.11 (s, 1H), 8.04 (d, J=7.8 Hz, 1H), 7.94 (d, J=11.5 Hz, 1H), 7.52 (t, J=8.2 Hz, 1H), 7.15 (s, 1H), 6.43 (s, 1H), 4.56 (d, J=6.2 Hz, 2H), 4.15 (t, J=5.3 Hz, 2H), 3.92 (s, 3H), 2.80 (t, J=5.0 Hz, 2H), 2.01 (br. s, 2H), 1.82 (br. s, 2H); LC/MS: m/z calculated for C₂₅H₂₃FN₈O([M+H]⁺): 471.5 Found: 471.2

### Example 48

### 5-tert-Butyl-isoxazole-3-carboxylicacid 2-fluoro-4-[6-(1-methyl-1H-pyrazol-4-yl)-7H-pyrrolo[2,3-d]pyrimidin-4-yl]-benzylamide

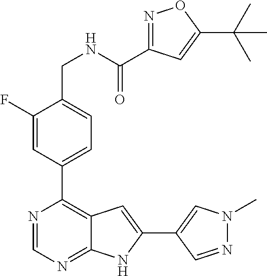 [SMILES: Cn1cc(-c2cc3c(-c4ccc(CNC(=O)c5cc(C(C)(C)C)on5)c(F)c4)ncnc3[nH]2)cn1]

In a 20 mL scintillation vial, {2-fluoro-4-[6-(1-methyl-1H-pyrazol-4-yl)-7H-pyrrolo[2,3-d]pyrimidin-4-yl]-benzyl}-carbamic acid tert-butyl ester (75 mg, 0.178 mmol, Eq: 1.00) was combined with 2 mL DCM and 2 mL TFA. The solution was stirred at room temperature for 30 minutes. The solvent was removed under reduced pressure. The crude material was dissolved in DMF (2 mL). 5-tert-butylisoxazole-3-carboxylic acid (33.0 mg, 0.195 mmol, Eq: 1.1), DIPEA (0.124 mL, 0.71 mmol, Eq: 4.00) and HATU (74 mg, 0.195 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was purified by column chromatography (silica gel, 0-10% MeOH in DCM). The title compound was obtained as a yellow solid (35 mg, 42% yield). LC/MS: m/z calculated for C₂₅H₂₄FN₇O₂([M+H]⁺): 474.5 Found: 474.3

### Example 49

### 3-tert-Butyl-[1,2,4]oxadiazole-5-carboxylic acid 2-fluoro-4-[6-(1-methyl-1H-pyrazol-4-yl)-7H-pyrrolo[2,3-d]pyrimidin-4-yl]-benzylamide

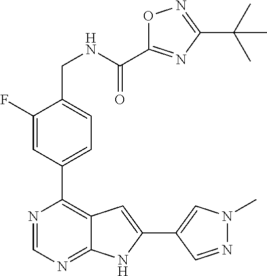 [SMILES: Cn1cc(-c2cc3c(-c4ccc(CNC(=O)c5nc(C(C)(C)C)no5)c(F)c4)ncnc3[nH]2)cn1]

In a 20 mL scintillation vial, {2-Fluoro-4-[6-(1-methyl-1H-pyrazol-4-yl)-7H-pyrrolo[2,3-d]pyrimidin-4-yl]-benzyl}-carbamic acid tert-butyl ester (150 mg, 0.355 mmol, Eq: 1.00) was combined with 3 mL DCM and 3 mL TFA. The solution was stirred at room temperature for 30 minutes. The solvent was removed under reduced pressure. The crude material was dissolved in DMF (4 mL). 3-tert-Butyl-1,2,4-oxadiazole-5-carboxylic acid (66.5 mg, 0.391 mmol, Eq: 1.1), DIPEA (0.25 mL, 1.42 mmol, Eq: 4.00) and bromotripyrrolidin-1-ylphosphonium (Pybrop) (182 mg, 0.391 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was preparative HPLC (10-100% acetonitrile-water). The title compound was obtained as a yellow solid (20 mg, 12% yield). LC/MS: m/z calculated for C₂₄H₂₃FN₈O₂([M+H]⁺): 475.5 Found: 475.2

### Example 50

### {2-Fluoro-4-[6-(1-methyl-1H-pyrazol-4-yl)-7H-pyrrolo[2,3-d]pyrimidin-4-yl]-benzyl}-carbamic acid tert-butyl ester

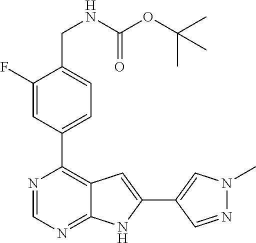 [SMILES: Cn1cc(-c2cc3c(-c4ccc(CNC(=O)OC(C)(C)C)c(F)c4)ncnc3[nH]2)cn1]

In a 20 mL sealable microwave tube, 7-benzenesulfonyl-4-chloro-6-(1-methyl-1H-pyrazol-4-yl)-7H-pyrrolo[2,3-d]pyrimidine (500 mg, 1.34 mmol, Eq: 1.00), [2-fluoro-4-(4,4,5,5-tetramethyl-[1,3,2]dioxaborolan-2-yl)-benzyl]-carbamic acid tert-butyl ester (541 mg, 1.61 mmol, Eq: 1.2) and potassium carbonate (739 mg, 5.35 mmol, Eq: 4.00) in 5 mL of water were combined with DME (10 mL). Pd(PPh₃)₄ (375 mg, 0.325 mmol, Eq: 0.1) was added. The reaction mixture was heated in a microwave at 160° C. for 60 minutes. The solution was washed with EtOAc and brine. The combined organic phases were dried over anhydrous sodium sulfate then the solvent was removed under reduced pressure. The crude material was purified by column chromatography (silica, 0-10% MeOH in DCM). The title compound was obtained as a yellow solid (150 mg, 27% yield). LC/MS: m/z calculated for C₂₂H₂₃FN₆O₂([M+H]⁺): 423.4 Found: 423.2

### Example 51

### N-{2-Fluoro-4-[6-(1-methyl-1H-pyrazol-4-yl)-7H-pyrrolo[2,3-d]pyrimidin-4-yl]-benzyl}-benzamide

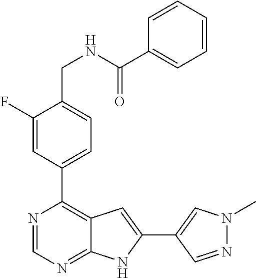 [SMILES: Cn1cc(-c2cc3c(-c4ccc(CNC(=O)c5ccccc5)c(F)c4)ncnc3[nH]2)cn1]

In a 20 mL scintillation vial, {2-Fluoro-4-[6-(1-methyl-1H-pyrazol-4-yl)-7H-pyrrolo[2,3-d]pyrimidin-4-yl]-benzyl}-carbamic acid tert-butyl ester (100 mg, 0.237 mmol, Eq: 1.00) was combined with 2 mL DCM and 2 mL TFA. The solution was stirred at room temperature for 30 minutes. The solvent was removed under reduced pressure. The crude material was dissolved in DMF (2 mL). Benzoic acid (32 mg, 0.26 mmol, Eq: 1.1), DIPEA (0.165 mL, 0.95 mmol, Eq: 4.00) and HATU (99 mg, 0.26 mmol, Eq: 1.1) were added. The reaction mixture was stirred overnight. The resulting solution was diluted with 10 mL of water and 5 mL of EtOAc. The solution was stirred at room temperature for an additional 30 minutes. The organic phases were extracted and dried over anhydrous sodium sulfate. The solvent was removed under reduced pressure. The resulting solid was purified by column chromatography (silica gel, 0-10% MeOH in DCM). The title compound was obtained as a yellow solid (8 mg, 8% yield). LC/MS: m/z calculated for C₂₄H₁₉FN₆O([M+H]⁺): 427.5 Found: 427.1

### Biological Examples

### Bruton's Tyrosine Kinase (Btk) Inhibition Assay

The assay is a capture of radioactive ³³P phosphorylated product through filtration. The interactions of Btk, biotinylated SH₂ peptide substrate (Src homology), and ATP lead to phosphorylation of the peptide substrate. Biotinylated product is bound streptavidin sepharose beads. All bound, radiolabeled products are detected by scintillation counter.

Plates assayed are 96-well polypropylene (Greiner) and 96-well 1.2 μm hydrophilic PVDF filter plates (Millipore). Concentrations reported here are final assay concentrations: 10-100 μM compounds in DMSO (Burdick and Jackson), 5-10 nM Btk enzyme (His-tagged, full-length), 30 μM peptide substrate (Biotin-Aca-AAAEEIYGEI-NH₂), 100 μM ATP (Sigma), 8 mM imidazole (Sigma, pH 7.2), 8 mM glycerol-2-phosphate (Sigma), 200 μM EGTA (Roche Diagnostics), 1 mM MnCl₂ (Sigma), 20 mM MgCl₂ (Sigma), 0.1 mg/ml BSA (Sigma), 2 mM DTT (Sigma), 1 μCi ³³P ATP (Amersham), 20% streptavidin sepharose beads (Amersham), 50 mM EDTA (Gibco), 2 M NaCl (Gibco), 2 M NaCl w/1% phosphoric acid (Gibco), microscint-20 (Perkin Elmer).

IC₅₀ determinations are calculated from 10 data points per compound utilizing data produced from a standard 96-well plate assay template. One control compound and seven unknown inhibitors were tested on each plate and each plate was run twice. Typically, compounds were diluted in half-log starting at 100 μM and ending at 3 nM. The control compound was staurosporine. Background was counted in the absence of peptide substrate. Total activity was determined in the presence of peptide substrate. The following protocol was used to determine Btk inhibition.

1) Sample preparation: The test compounds were diluted at half-log increments in assay buffer (imidazole, glycerol-2-phosphate, EGTA, MnCl₂, MgCl₂, BSA).
2) Bead preparation
a.) rinse beads by centrifuging at 500 g
b.) reconstitute the beads with PBS and EDTA to produce a 20% bead slurry
   3) Pre-incubate reaction mix without substrate (assay buffer, DTT, ATP, ³³P ATP) and mix with substrate (assay buffer, DTT, ATP, ³³P ATP, peptide substrate) 30° C. for 15 min.
   4) To start assay, pre-incubate 10 μL Btk in enzyme buffer (imidazole, glycerol-2-phosphate, BSA) and 10 μL of test compounds for 10 min at RT.
   5) Add 30 μL reaction mixture without or with substrate to Btk and compounds.
   6) Incubate 50 μL total assay mix for 30 min at 30° C.
   7) Transfer 40 μL of assay to 150 μL bead slurry in filter plate to stop reaction.
   8) Wash filter plate after 30 min, with following steps
a. 3×250 μL NaCl
b. 3×250 μL NaCl containing 1% phosphoric acid
c. 1×250 μL H₂O
   9) Dry plate for 1 h at 65° C. or overnight at RT
   10) Add 50 μL microscint-20 and count ³³P cpm on scintillation counter.

Calculate percent activity from raw data in cpm percent activity=(sample−bkg)/(total activity−bkg)×100

Calculate IC₅₀ from percent activity, using one-site dose response sigmoidal model y=A+((B−A)/(1+((x/C)^{D}))))

x=cmpd conc, y=% activity, A=min, B=max, C=IC₅₀, D=1 (hill slope)

### Bruton's Tyrosine Kinase (BTK) Inhibition TR-FRET (Time Resolved FRET) Assay

This BTK competition assay measures compound potency (IC50) for the inactivated state of Bruton's Tyrosine Kinase using FRET (Forster/Fluorescence Resonance Energy Transfer) technology. The BTK-Eu complex was incubated on ice one hour prior to use at a starting concentration of 50 nM BTK-Bioease™: 10 nM Eu-streptavidin (Perkin-Elmer Catalog# AD0062). The assay buffer consisted of 20 mM HEPES (pH 7.15), 0.1 mM DTT, 10 mM MgCl₂, 0.5 mg/ml BSA with 3% Kinase Stabilizer (Fremont Biosolutions, Catalog # STB-K02). After 1 h, the reaction mixture from above was diluted 10 fold in assay buffer to make 5 nM BTK: 1 nM Eu-Streptavidin complex (donor fluorophore). 180 of a mixture of 0.11 nM BTK-Eu and 0.11 nM Kinase Tracer 178 (Invitrogen, Catalog # PV5593,) with BTK-Eu alone as no negative control, was then dispensed into 384-well flat bottom plates (Greiner, 784076). Compounds to be tested in assay were prepared as 10× concentrations and serial dilution in half-log increments was performed in DMSO so as to generate 10 point curves. To initiate the FRET reaction, compounds prepared as 10× stock in DMSO was added to the plates and the plates were incubated 18-24 h at 14° C.

After the incubation the plates were read on a BMG Pherastar Fluorescent plate reader (or equivalent) and used to measure the emission energy from the europium donor fluorophore (620 nm emission) and the FRET (665 nm emission). The negative control well values were averaged to obtain the mean minimum. The positive “no inhibitor” control wells were averaged to obtain the mean maximum. Percent of maximal FRET was calculated using following equation:

% max FRET=100×[(FSR_{cmpd}−FSR_{mean min})/(FSR_{mean max}−FSR_{mean min})]

where FSR=FRET Signal ratio. % Max FRET curves were plotted in Activity Base (Excel) and the IC50(%), hill slope, z′ and % CV were determined. The mean IC50 and standard deviation will be derived from duplicate curves (singlet inhibition curves from two independent dilutions) using Microsoft Excel.

Representative compound data for this assay are listed below in Table II.

TABLE II

| | FRET IC50 | HWB IC50 |
|---|---|---|
| Compound | (μM) | (μM) |
| I-1 | 34.7 | |
| I-2 | >100 | |
| I-3 | >100 | |
| I-4 | 16.3 | |
| I-5 | 33.1 | |
| I-6 | 10.4 | |
| I-7 | 16.4 | |
| I-8 | >100 | |
| I-9 | 0.481 | |
| I-10 | 5.68 | |
| I-11 | 12.2 | |
| I-12 | 0.121 | |
| I-13 | >100 | |
| I-14 | 0.129 | |
| I-15 | 1.53 | |
| I-16 | 0.687 | |
| I-17 | 0.098 | 16.2 |
| I-18 | 0.137 | |
| I-19 | 0.137 | |
| I-20 | 0.062 | 3 |
| I-21 | 0.007 | 1.4 |
| I-22 | 0.008 | 5.48 |
| I-23 | 0.007 | 1.4 |
| I-24 | 0.065 | |
| I-25 | 0.044 | 28.9 |
| I-26 | 0.001 | 0.608 |
| I-27 | 0.002 | 0.812 |
| I-28 | 0.004 | 2.54 |
| I-29 | 0.003 | 0.47 |
| I-30 | 0.0007 | 1.04 |
| I-31 | 0.02 | 6.05 |
| I-32 | 0.008 | |
| I-33 | 0.001 | 0.129 |
| I-34 | 0.0002 | 0.076 |
| I-35 | 0.008 | 5.5 |
| I-36 | 0.004 | 6.74 |
| I-37 | 0.0003 | 0.655 |
| I-38 | 0.003 | 3.27 |
| I-39 | 0.0002 | 0.028 |
| I-40 | 0.0006 | 0.052 |
| I-41 | 0.0006 | 0.154 |
| I-42 | 0.0005 | 0.23 |
| I-43 | 0.0006 | 0.276 |
| I-44 | 0.00052 | 0.188 |
| I-45 | 0.0006 | 0.222 |
| I-46 | 0.001 | >50 |
| I-47 | 0.0032 | 0.674 |
| I-48 | 0.00019 | 0.076 |
| I-49 | 0.00011 | 0.002 |
| I-50 | 1.49 | |
| I-51 | 0.026 | |

### Inhibition of B Cell Activation in Whole Blood Measured by CD69 Expression

A procedure to test the ability of Btk inhibitors to suppress B cell receptor-mediated activation of B cells in human blood is as follows:

Human whole blood (HWB) is obtained from healthy volunteers, with the following restrictions: 24 hr drug-free, non-smokers. Blood is collected by venipuncture into Vacutainer tubes anticoagulated with sodium heparin. Test compounds are diluted to ten times the desired starting drug concentration in PBS (20×), followed by three-fold serial dilutions in 10% DMSO in PBS to produce a nine point dose-response curve. 5.5 μl of each compound dilution is added in duplicate to a 2 ml 96-well V bottom plate (Analytical Sales and Services, #59623-23); 5.5 μl of 10% DMSO in PBS is added to control and no-stimulus wells. HWB (100 μl) is added to each well, and after mixing the plates are incubated at 37 C, 5% CO₂, 100% humidity for 30 minutes. Goat F(ab′)2 anti-human IgM (Southern Biotech, #2022-14) (10 μl of a 500 μg/ml solution, 50 μg/ml final concentration) is added to each well (except the no-stimulus wells) with mixing and the plates are incubated for an additional 20 hours.

At the end of the 20 hour incubation, samples are incubated with florescent-probe-labeled anti-bodies (15 μl PE Mouse anti-Human CD20, BD Pharmingen, #555623, and/or 20 ul APC Mouse anti-Human CD69, BD Pharmingen #555533) for 30 minutes, at 37 C, 5% CO₂, 100% humidity. Included are induced control, unstained and single stains for compensation adjustments and initial voltage settings. Samples are then lysed with 1 ml of 1× Pharmingen Lyse Buffer (BD Pharmingen #555899), and plates are centrifuged at 1800 rpm for 5 minutes. Supernatants are removed via suction and the remaining pellets are lysed again with another 1 ml of 1× Pharmingen Lyse Buffer, and plates are spun down as before. Supernatants are aspirated and remaining pellets are washed in FACs buffer (PBS+1% FBS). After a final spin, the supernantants are removed and pellets are resuspended in 180 μl of FACs buffer. Samples are transferred to a 96 well plate suitable to be run on the HTS 96 well system on the BD LSR II flow cytometer.

Using appropriate excitation and emission wavelengths for the fluorophores used, data are acquired and percent positive cell values are obtained using Cell Quest Software. Results are initially analyzed by FACS analysis software (Flow Jo). The IC50 for test compounds is defined as the concentration which decreases by 50% the percentage of CD69-positive cells that are also CD20-positive after stimulation by anti-IgM (average of 8 control wells, after subtraction of the average of 8 wells for the no-stimulus background). The IC50 values are calculated using XLfit software version 3, equation 201.

### Inhibition of B-Cell Activation—B Cell FLIPR Assay in Ramos Cells

Inhibition of B-cell activation by compounds of the present invention is demonstrated by determining the effect of the test compounds on anti-IgM stimulated B cell responses.

The B cell FLIPR assay is a cell based functional method of determining the effect of potential inhibitors of the intracellular calcium increase from stimulation by an anti-IgM antibody. Ramos cells (human Burkitt's lymphoma cell line. ATCC-No. CRL-1596) were cultivated in Growth Media (described below). One day prior to assay, Ramos cells were resuspended in fresh growth media (same as above) and set at a concentration of 0.5×10⁶/mL in tissue culture flasks. On day of assay, cells are counted and set at a concentration of 1×10⁶/mL1 in growth media supplemented with 1 μM FLUO-3AM(TefLabs Cat-No. 0116, prepared in anhydrous DMSO and 10% Pluronic acid) in a tissue culture flask, and incubated at 37° C. (4% CO₂) for one h. To remove extracellular dye, cells were collected by centrifugation (5 min, 1000 rpm), resuspended in FLIPR buffer (described below) at 1×10⁶ cells/mL and then dispensed into 96-well poly-D-lysine coated black/clear plates (BD Cat-No. 356692) at 1×10⁵ cells per well. Test compounds were added at various concentrations ranging from 100 μM to 0.03 μM (7 concentrations, details below), and allowed to incubate with cells for 30 min at RT. Ramos cell Ca²⁺ signaling was stimulated by the addition of 10 μg/mL anti-IgM (Southern Biotech, Cat-No. 2020-01) and measured on a FLIPR (Molecular Devices, captures images of 96 well plates using a CCD camera with an argon laser at 480 nM excitation).

### Media/Buffers:

Growth Medium: RPMI 1640 medium with L-glutamine (Invitrogen, Cat-No. 61870-010), 10% Fetal Bovine Serum (FBS, Summit Biotechnology Cat-No. FP-100-05); 1 mM Sodium Pyruvate (Invitrogen Cat. No. 11360-070).

FLIPR buffer: HBSS (Invitrogen, Cat-No. 141175-079), 2 mM CaCl₂ (Sigma Cat-No. C-4901), HEPES (Invitrogen, Cat-No. 15630-080), 2.5 mM Probenecid (Sigma, Cat-No. P-8761), 0.1% BSA (Sigma, Cat-No.A-7906), 11 mM Glucose (Sigma, Cat-No.G-7528)

### Compound Dilution Details:

In order to achieve the highest final assay concentration of 100 μM, 24 μL of 10 mM compound stock solution (made in DMSO) is added directly to 576 μL of FLIPR buffer. The test compounds are diluted in FLIPR Buffer (using Biomek 2000 robotic pipettor) resulting in the following dilution scheme: vehicle, 1.00×10⁻⁴ M, 1.00×10⁻⁵, 3.16×10⁻⁶, 1.00×10⁻⁶, 3.16×10⁻⁷, 1.00×10⁻⁷, 3.16×10-8.

### Assay and Analysis:

Intracellular increases in calcium were reported using a max—min statistic (subtracting the resting baseline from the peak caused by addition of the stimulatory antibody using a Molecular Devices FLIPR control and statistic exporting software. The IC₅₀ was determined using a non-linear curve fit (GraphPad Prism software).

Mouse Collagen-Induced Arthritis (mCIA)

On day 0 mice are injected at the base of the tail or several spots on the back with an emulsion of Type II Collagen (i.d.) in Complete Freund's adjuvant (CFA). Following collagen immunization, animals will develop arthritis at around 21 to 35 days. The onset of arthritis is synchronized (boosted) by systemic administration of collagen in Incomplete Freund's adjuvant (IFA; i.d.) at day 21. Animals are examined daily after day 20 for any onset of mild arthritis (score of 1 or 2; see score description below) which is the signal to boost. Following boost, mice are scored and dosed with candidate therapeutic agents for the prescribed time (typically 2-3 weeks) and dosing frequency, daily (QD) or twice-daily (BID).

Rat Collagen-Induced Arthritis (rCIA)

On day 0, rats are injected with an emulsion of Bovine Type II Collagen in Incomplete Freund's adjuvant (IFA) is injected intradermally (i.d.) on several locations on the back. A booster injection of collagen emulsion is given around day 7, (i.d.) at the base of the tail or alternative sites on the back. Arthritis is generally observed 12-14 days after the initial collagen injection. Animals may be evaluated for the development of arthritis as described below (Evaluation of arthritis) from day 14 onwards. Animals are dosed with candidate therapeutic agents in a preventive fashion starting at the time of secondary challenge and for the prescribed time (typically 2-3 weeks) and dosing frequency, daily (QD) or twice-daily (BID).

### Evaluation of Arthritis:

In both models, developing inflammation of the paws and limb joints is quantified using a scoring system that involves the assessment of the 4 paws following the criteria described below:

Scoring: 1=swelling and/or redness of paw or one digit.
2=swelling in two or more joints.
3=gross swelling of the paw with more than two joints involved.
4=severe arthritis of the entire paw and digits.

Evaluations are made on day 0 for baseline measurement and starting again at the first signs or swelling for up to three times per week until the end of the experiment. The arthritic index for each mouse is obtained by adding the four scores of the individual paws, giving a maximum score of 16 per animal.

### Rat In Vivo Asthma Model

Male Brown-Norway rats are sensitized i.p. with 100 μg of OA (ovalbumin) in 0.2 ml alum once every week for three weeks (day 0, 7, and 14). On day 21 (one week following last sensitization), the rats are dosed q.d. with either vehicle or compound formulation subcutaneously 0.5 hour before OA aerosol challenge (1% OA for 45 minutes) and terminated 4 or 24 hours after challenge. At time of sacrifice, serum and plasma are collected from all animals for serology and PK, respectively. A tracheal cannula is inserted and the lungs are lavaged 3× with PBS. The BAL fluid is analyzed for total leukocyte number and differential leukocyte counts. Total leukocyte number in an aliquot of the cells (20-100 μl) is determined by Coulter Counter. For differential leukocyte counts, 50-200 μl of the sample is centrifuged in a Cytospin and the slide stained with Diff-Quik. The proportions of monocytes, eosinophils, neutrophils and lymphocytes are counted under light microscopy using standard morphological criteria and expressed as a percentage. Representative inhibitors of Btk show decreased total leucocyte count in the BAL of OA sensitized and challenged rats as compared to control levels.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A compound of Formula I, 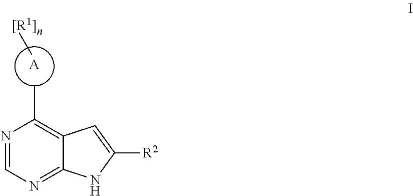
wherein:
A is phenyl or piperidinyl;
each R¹ is independently halo, lower alkyl, CH₂NHC(═O)R^{1′}, CH₂N(CH₃)C(═O)R^{1′}, CH₂NHC(═O)CH₂NHR^{1′}, CH₂R^{1′}, or CH₂NHR^{1′};
n is 0, 1, or 2;
R^{1′} is phenyl, unsaturated or partially unsaturated bicyclic or monocyclic heteroaryl, or heterocycloalkyl, optionally substituted with one or more R^{1″};
each R^{1″} is independently lower alkyl, halo, cycloalkyl, heterocycloalkyl, loweralkyl heterocycloalkyl, oxo, cyano loweralkyl, hydroxyl loweralkyl, or lower alkoxy;
R² is H, R³ or R⁴;
R³ is C(═O)OR^{3′}, C(═O)R^{3′}, or C(═O)NH(CH₂)₂R^{3′};
R^{3′} is H, lower alkyl, heterocycloalkyl, amino, or OH;
R⁴ is lower alkyl or heteroaryl, optionally substituted with one or more R^{4′}; and
R^{4′} is methyl, hydroxyl, amino, CH₂—CH₂N(CH₃)₂, OC(═O) CH₂CH₃, CH₂C(═O)OH, CH₂CH₂OH or C(═O)OH;
or a pharmaceutically acceptable salt thereof.

2. The compound of claim 1, wherein A is phenyl, R² is H and n is 1.

3. The compound of claim 1, wherein R¹ is halo.

4. The compound of claim 1, wherein R² is H and n is 2.

5. The compound of claim 4, wherein one R¹ is halo or lower alkyl.

6. The compound of claim 1, wherein R¹ is CH₂NHC(═O)R^{1′}, CH₂NHC(═O)CH₂NHR^{1′} or CH₂NHR^{1′}.

7. The compound of claim 1, wherein n is 2, one R¹ is CH₂NHC(═O)R^{1′} and R² is C(═O)OR^{3′}, C(═O)R^{3′}, or C(═O)NH(CH₂)₂R^{3′}.

8. The compound of claim 1, wherein n is 2, one R¹ is CH₂NHC(═O)R^{1′}.

9. The compound of claim 1, wherein n is 2, one R¹ is CH₂NHC(═O)R^{1′} and R² is lower alkyl or heteroaryl.

10. The compound of claim 1, wherein R^{1′} is tert butyl or halo.

11. The compound of claim 1, wherein one R¹ is fluorine and R^{1′} is tert butyl.

12. The compound of claim 1 selected from the group consisting of:
4-(4-Chloro-phenyl)-7H-pyrrolo[2,3-d]pyrimidine;
4-(3-Chloro-phenyl)-7H-pyrrolo[2,3-d]pyrimidine;
4-(2-Chloro-phenyl)-7H-pyrrolo[2,3-d]pyrimidine;
4-(3-Fluoro-4-methyl-phenyl)-7H-pyrrolo[2,3-d]pyrimidine;
4-(2,4-Dimethyl-phenyl)-7H-pyrrolo[2,3-d]pyrimidine;
4-(3,4-Dimethyl-phenyl)-7H-pyrrolo[2,3-d]pyrimidine;
4-p-Tolyl-7H-pyrrolo[2,3-d]pyrimidine;
4-(3-Chloro-4-methyl-phenyl)-7H-pyrrolo[2,3-d]pyrimidine;
4-tert-Butyl-N-[4-(7H-pyrrolo[2,3-d]pyrimidin-4-yl)-benzyl]-benzamide;
3-Chloro-N-[4-(7H-pyrrolo[2,3-d]pyrimidin-4-yl)-benzyl]-benzamide;
2-(3-Chloro-phenylamino)-N-[4-(7H-pyrrolo[2,3-d]pyrimidin-4-yl)-benzyl]-acetamide;
4-tert-Butyl-N-[2-fluoro-4-(7H-pyrrolo[2,3-d]pyrimidin-4-yl)-benzyl]-benzamide;
4-{4-[(4-tert-Butyl-benzoylamino)-methyl]-3-fluoro-phenyl}-7H-pyrrolo[2,3-d]pyrimidine-6-carboxylic acid tert-butyl ester;
4-(4-((4-tert-butylbenzamido)methyl)-3-fluorophenyl)-7H-pyrrolo[2,3-d]pyrimidine-6-carboxylic acid;
4-tert-butyl-N-(2-fluoro-4-(6-(morpholine-4-carbonyl)-7H-pyrrolo[2,3-d]pyrimidin-4-yl)benzyl)benzamide;
4-{4-[(4-tert-Butyl-benzoylamino)-methyl]-3-fluoro-phenyl}-7H-pyrrolo[2,3-d]pyrimidine-6-carboxylic acid dimethylamide;
4-{4-[(4-tert-Butyl-benzoylamino)-methyl]-3-fluoro-phenyl}-7H-pyrrolo[2,3-d]pyrimidine-6-carboxylic acid methylamide;
4-{4-[(4-tert-Butyl-benzoylamino)-methyl]-3-fluoro-phenyl}-7H-pyrrolo[2,3-d]pyrimidine-6-carboxylic acid (2-hydroxy-ethyl)-amide;
4-{4-[(4-tert-Butyl-benzoylamino)-methyl]-3-fluoro-phenyl}-7H-pyrrolo[2,3-d]pyrimidine-6-carboxylic acid (2-dimethylamino-ethyl)-amide;
4-tert-Butyl-N-{1-[6-(1-methyl-1H-pyrazol-4-yl)-7H-pyrrolo[2,3-d]pyrimidin-4-yl]-piperidin-4-ylmethyl}-benzamide;
4-tert-Butyl-N-{4-[6-(1-methyl-1H-pyrazol-4-yl)-7H-pyrrolo[2,3-d]pyrimidin-4-yl]-benzyl}-benzamide;
4-Cyclopropyl-N-{4-[6-(1-methyl-1H-pyrazol-4-yl)-7H-pyrrolo[2,3-d]pyrimidin-4-yl]-benzyl}-benzamide;
4-Isopropyl-N-{4-[6-(1-methyl-1H-pyrazol-4-yl)-7H-pyrrolo[2,3-d]pyrimidin-4-yl]-benzyl}-benzamide;
N-{4-[6-(1-Methyl-1H-pyrazol-4-yl)-7H-pyrrolo[2,3-d]pyrimidin-4-yl]-benzyl}-4-oxetan-3-yl-benzamide;
4-(3-Methyl-oxetan-3-yl)-N-{4-[6-(1-methyl-1H-pyrazol-4-yl)-7H-pyrrolo[2,3-d]pyrimidin-4-yl]-benzyl}-benzamide;
4,5,6,7-Tetrahydro-benzo[b]thiophene-2-carboxylic acid 4-[6-(1-methyl-1H-pyrazol-4-yl)-7H-pyrrolo[2,3-d]pyrimidin-4-yl]-benzylamide;
4-tert-Butyl-N-{2-fluoro-4-[6-(1-methyl-1H-pyrazol-4-yl)-7H-pyrrolo[2,3-d]pyrimidin-4-yl]-benzyl}-benzamide;
6-tert-Butyl-N-{2-fluoro-4-[6-(1-methyl-1H-pyrazol-4-yl)-7H-pyrrolo[2,3-d]pyrimidin-4-yl]-benzyl}-nicotinamide;
5-Methyl-thiophene-2-carboxylic acid 2-fluoro-4-[6-(1-methyl-1H-pyrazol-4-yl)-7H-pyrrolo[2,3-d]pyrimidin-4-yl]-benzylamide;
4-tert-Butyl-N-(2-fluoro-4-{6-[1-(2-hydroxy-ethyl)-1H-pyrazol-4-yl]-7H-pyrrolo[2,3-d]pyrimidin-4-yl}-benzyl)-benzamide;
4-tert-Butyl-N-{2-fluoro-4-[6-(1-methyl-1H-pyrazol-4-yl)-7H-pyrrolo[2,3-d]pyrimidin-4-yl]-benzyl}-N-methyl-benzamide;
5-Methyl-thiophene-2-carboxylic acid {2-fluoro-4-[6-(1-methyl-1H-pyrazol-4-yl)-7H-pyrrolo[2,3-d]pyrimidin-4-yl]-benzyl}-methyl-amide;
2-tert-Butyl-5-{2-fluoro-4-[6-(1-methyl-1H-pyrazol-4-yl)-7H-pyrrolo[2,3-d]pyrimidin-4-yl]-benzyl}-4,5-dihydro-thieno[2,3-c]pyrrol-6-one;
5-tert-Butyl-isoxazole-3-carboxylic acid 2-fluoro-4-[6-(1-methyl-1H-pyrazol-4-yl)-7H-pyrrolo[2,3-d]pyrimidin-4-yl]-benzylamide;
N-{2-Fluoro-4-[6-(1-methyl-1H-pyrazol-4-yl)-7H-pyrrolo[2,3-d]pyrimidin-4-yl]-benzyl}-4-(3-methyl-oxetan-3-yl)-benzamide;
4-(Cyano-dimethyl-methyl)-N-{2-fluoro-4-[6-(1-methyl-1H-pyrazol-4-yl)-7H-pyrrolo[2,3-d]pyrimidin-4-yl]-benzyl}-benzamide;
4,5,6,7-Tetrahydro-benzo[b]thiophene-2-carboxylic acid 2-fluoro-4-[6-(1-methyl-1H-pyrazol-4-yl)-7H-pyrrolo[2,3-d]pyrimidin-4-yl]-benzylamide;
N-{2-Fluoro-4-[6-(1-methyl-1H-pyrazol-4-yl)-7H-pyrrolo[2,3-d]pyrimidin-4-yl]-benzyl}-4-(1-hydroxy-1-methyl-ethyl)-benzamide;
3-tert-Butyl-isoxazole-5-carboxylic acid 2-fluoro-4-[6-(1-methyl-1H-pyrazol-4-yl)-7H-pyrrolo[2,3-d]pyrimidin-4-yl]-benzyl amide
3-tert-Butoxy-azetidine-1-carboxylic acid 2-fluoro-4-[6-(1-methyl-1H-pyrazol-4-yl)-7H-pyrrolo[2,3-d]pyrimidin-4-yl]-benzylamide;
1,3-Dihydro-isoindole-2-carboxylic acid 2-fluoro-4-[6-(1-methyl-1H-pyrazol-4-yl)-7H-pyrrolo[2,3-d]pyrimidin-4-yl]-benzylamide;
4-tert-Butyl-N-(4-{6-[1-(2-dimethylamino-ethyl)-1H-pyrazol-4-yl]-7H-pyrrolo[2,3-d]pyrimidin-4-yl}-2-fluoro-benzyl)-benzamide;
3-tert-Butoxy-azetidine-1-carboxylic acid 4-{6-[1-(2-dimethylamino-ethyl)-1H-pyrazol-4-yl]-7H-pyrrolo[2,3-d]pyrimidin-4-yl}-2-fluoro-benzylamide;
1,3-Dihydro-isoindole-2-carboxylic acid 4-{6-[1-(2-dimethylamino-ethyl)-1H-pyrazol-4-yl]-7H-pyrrolo[2,3-d]pyrimidin-4-yl}-2-fluoro-benzylamide;
[4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid ethyl ester;
[4-(4-{4-[(4-tert-Butyl-benzoylamino)-methyl]-3-fluoro-phenyl}-7H-pyrrolo[2,3-d]pyrimidin-6-yl)-pyrazol-1-yl]-acetic acid;
N-(2-fluoro-4-(6-(1-methyl-1H-pyrazol-4-yl)-7H-pyrrolo[2,3-d]pyrimidin-4-yl)benzyl)-4,5,6,7-tetrahydropyrazolo[1,5-a]pyridine-2-carboxamide;
5-tert-Butyl-isoxazole-3-carboxylicacid 2-fluoro-4-[6-(1-methyl-1H-pyrazol-4-yl)-7H-pyrrolo[2,3-d]pyrimidin-4-yl]-benzylamide;
3-tert-Butyl-[1,2,4]oxadiazole-5-carboxylic acid 2-fluoro-4-[6-(1-methyl-1H-pyrazol-4-yl)-7H-pyrrolo[2,3-d]pyrimidin-4-yl]-benzylamide;
{2-Fluoro-4-[6-(1-methyl-1H-pyrazol-4-yl)-7H-pyrrolo[2,3-d]pyrimidin-4-yl]-benzyl}-carbamic acid tert-butyl ester; and
N-{2-Fluoro-4-[6-(1-methyl-1H-pyrazol-4-yl)-7H-pyrrolo[2,3-d]pyrimidin-4-yl]-benzyl}-benzamide.

13. A method for treating an inflammatory and/or autoimmune condition comprising administering to a patient in need thereof a therapeutically effective amount of the compound of claim 1.

14. A method for treating an inflammatory condition comprising administering to a patient in need thereof a therapeutically effective amount of the compound of claim 1.

15. A method for treating rheumatoid arthritis comprising administering to a patient in need thereof a therapeutically effective amount of the compound of claim 1.

16. A method for treating asthma comprising administering to a patient in need thereof a therapeutically effective amount of the compound of claim 1.

17. A pharmaceutical composition, comprising a therapeutically effective amount of a compound of claim 1, admixed with at least one pharmaceutically acceptable carrier, excipient or diluent.

18-22. (canceled)
